SEQUENCE LISTING (2 sequences)

SEQ ID NO: 1 - DNA, 7330 bp, Artificial sequence
  Synthetic
cgtaactataacggtcctaaggtagcgaaattgttctactagaggtgacttccagaagatgctaaatctcttcaaaaaaaaaaccccaaaaccgaaaaacagtcactgcgacttcttactccctgggttagacagcattggagtgcttgttcatgagctgtatttattcagctattagcccagcgtttcaattttagggcaaaagagaaccagacagggccaaaaagtcttttcatccttcgcctgggaacaccggcctccttactctgtaaatgtaactgtttctctggattagattttagtctttttttttttttttttttaagcactccatatattcgttccattatttaaagtgggcttggaggaagcgtgccgcatccttccgccctgtgcgtctgcttgtgtccgtctgaagcaggtccctttcggctctcgagtctgggtcgccaggacgcggggtggtggggaggagatgggggggaagagtcccctgcccctcgtgcgacgctgagcgccggcgtccgggccgcggaggcagagccggagccgtaagaactcgcctgcacctcgcgccgccagccgcggagtggagaaaggagggagggggtcggaggagggaaagtggggccgggcggggggtgcctgggaagccgggctgcgctgacgtcactgggcgcgcgattcgggctggagcgctttaaaaaacgagcgtgcgagcggagatgctgctccacaccgagcaggccgcctgcagcagcagcgctcacggcccctgcggcggctgcagccagggccgctcccgaggcccgcgtggcggcaggggcacggccgggccccgcgacgcttctccttggcgcaccgcgctgcaggcgcccgcggcgtcgcagaagcccgccgagctgcggctggcgcgccacggtccgccgcaagcccgacactgacctcccaccgcgccccgcacgcggccccccgcccgggcaccatgggacctgagccatccaagtctgctccagctcctaagaagggctctaagaaggccatcaccaaggcgcaaaagaaagatgggaaaaagcgaaagcgcagccgtaaggagagctactccatttatgtgtacaaggtactgaagcaggtccacccggacactggtatttcctccaaggctatgggcatcatgaattcgttcgtcaacgacattttcgagcggatcgccggtgaggcttcgcgcctggcccattacaacaaacgctcgaccatcacttccagggagatccagacggctgtgcgcctgctgctgccgggggagctggctaagcacgctgtgtccgagggaaccaaggcggttaccaagtacactagctccaaacctgcaggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctggcgcgccttggcgtgctcctgtccttgcccgtcctgttccagctggcggccggagtcggctcgccaaggcccagcgcgctgctgcgggggtgccccgcgcactgtcagtgcgagccggacggcaggatgctgctcagggtggactgctctgacctggggctttcggagctgccctccaacctcagcgttttcacctcctacctgtaagtgcgtcccccactccgctcccaaaaggggacagagggagggaggctggccctagaccaggctgggggctcctcggctcctgcctgctctggagggggcgggcgagtttgcaaaggatgtcttggccgggactgtttggggccctggaaaatgcacgtgtctcggggtagcacgcctgcactttatgggcccgcagaaaaacggcttcaagtgcaaccccgggaaagcgccggtgcgctgcagctttgcggtccagcttcaggcagggcgcagcgcgaatttctccgattgtagacttttgcctgcaaaatgggcatttgtgcccctgagcctggaacccaaggacagcctgcccagttggcccctgctccaacctccccttcttgtcctgtcgtgtttcaggaagagatggactgtaaaaagtttccccgaagaactccaacttgtctagctccagaggaacttactctgcatcccttgctccaacgtgtgatgcaacctttttttactcctctctgtctctgtctttctgcttctctgtctctctttctctaatgaagtgattaaaatgtggcaaccgcaaaccccgattagagctgccagaacgttatctttacctttggctgcccgagccgccctttgaagtgcccgcggccgcggtgtaatctttaaccatctcctcctcctcccagagaggaaagggaagtgggctgaaggcggcggggcgggtaaacagcggcgccaacacccaagtggagacttgaagctctctgtcatccctcggggatcgtcccaccccttttccccccaccacagcctccaaagccccaaccgtggagacgaggcagcggctcctttcgtaattctgtctctggaaaatccaggcccggagggtaacaggtggttgggtggatgaggtgacccaggaagaacctgactcttgggaaccgagataacgactctttctccctatgaaggatctggggctcgcttcctttgggcagagagaccaggggtcggggccactcgggaaaaggggcgcttgcgaccacaaggtcatgcgtttagattttaactagaggccctgtggcgagcccaccccaaacgctcgcaatcccagtttcggcttctctgatgtaccttggcaggcgctagttgggatgtttgtgaactcagccgttcctctgtttagttagggataacagggtaataagggcgaattccagcacactggcggccgttactagtggatccgagctcggtaccaagcttgatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtggccctcctcacgtgctattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgccccggcgtgtcaataatatcactctgtacatccacaaacagacgataacggctctctcttttataggtgtaaaccttaaactgccgtacgtataggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggccctctagatgcatgctcgagcggccgccagtgtgatggatatctgcagaattcgccctt

SEQ ID NO: 2 - DNA, 135400 bp, Artificial sequence
  Synthetic
acttaggtccaggaatctatatatttttaaaagttccacaggtggtcagccaggtttggggatcacttcttaagtgaaggagagtaaacatacacaaaggaaaaaaaaaatcttcccttgggtaaaattcagaagtttatatgggaaactcagcagaatgggctaagtgaaagaagcttaagagaggccttaaaagccagaagggttctccacagtcatgaagcaaggaggcattgtacgggagtgaaaactgtgcatttctgtgagttgaagtggcaggctggagctgtgactctttcagccactcactcaggcccgcagacaagatttgctcctcactgaggccctctgtccaagaaagccctgagtctttctcctcctttctcctgcaggctcccagcaggacacttctgaagagtgagtacgagcattttgcagtggtaatggttctcagagcccagtgcctctcacctggctagggctgctcttaacttagaaaatcctggaacacttaggatgaaaaggccgcggaacaaaagcaaacgcccatctcccatctcgctgtcctgtccgtcccgcattgttctactagaggtgacttccagaagatgctaaatctcttcaaaaaaaaaaccccaaaaccgaaaaacagtcactgcgacttcttactccctgggttagacagcattggagtgcttgttcatgagctgtatttattcagctattagcccagcgtttcaattttagggcaaaagagaaccagacagggccaaaaagtcttttcatccttcgcctgggaacaccggcctccttactctgtaaatgtaactgtttctctggattagattttagtcttttttttttttttttttaagcactccatatattcgttccattatttaaagtgggcttggaggaagcgtgccgcatccttccgccctgtgcgtctgcttgtgtccgtctgaagcaggtccctttcggctctcgagtctgggtcgccaggacgcggggtggtggggaggagatgggggggaagagtcccctgcccctcgtgcgacgctgagcgccggcgtccgggccgcggaggcagagccggagccgtaagaactcgcctgcacctcgcgccgccagccgcggagtggagaaaggagggagggggtcggaggagggaaagtggggccgggcggggggtgcctgggaagccgggctgcgctgacgtcactgggcgcgcgattcgggctggagcgctttaaaaaacgagcgtgcgagcggagatgctgctccacaccgagcaggccgcctgcagcagcagcgctcacggcccctgcggcggctgcagccagggccgctcccgaggcccgcgtggcggcaggggcacggccgggccccgcgacgcttctccttggcgcaccgcgctgcaggcgcccgcggcgtcgcagaagcccgccgagctgcggctggcgcgccacggtccgccgcaagcccgacactgacctcccaccgcgccccgcacgcggccccccgcccgggcaccatgggacctgagccatccaagtctgctccagctcctaagaagggctctaagaaggccatcaccaaggcgcaaaagaaagatgggaaaaagcgaaagcgcagccgtaaggagagctactccatttatgtgtacaaggtactgaagcaggtccacccggacactggtatttcctccaaggctatgggcatcatgaattcgttcgtcaacgacattttcgagcggatcgccggtgaggcttcgcgcctggcccattacaacaaacgctcgaccatcacttccagggagatccagacggctgtgcgcctgctgctgccgggggagctggctaagcacgctgtgtccgagggaaccaaggcggttaccaagtacactagctccaaacctgcaggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctggcgcgccttggcgtgctcctgtccttgcccgtcctgttccagctggcggccggagtcggctcgccaaggcccagcgcgctgctgcgggggtgccccgcgcactgtcagtgcgagccggacggcaggatgctgctcagggtggactgctctgacctggggctttcggagctgccctccaacctcagcgttttcacctcctacctgtaagtgcgtcccccactccgctcccaaaaggggacagagggagggaggctggccctagaccaggctgggggctcctcggctcctgcctgctctggagggggcgggcgagtttgcaaaggatgtcttggccgggactgtttggggccctggaaaatgcacgtgtctcggggtagcacgcctgcactttatgggcccgcagaaaaacggcttcaagtgcaaccccgggaaagcgccggtgcgctgcagctttgcggtccagcttcaggcagggcgcagcgcgaatttctccgattgtagacttttgcctgcaaaatgggcatttgtgcccctgagcctggaacccaaggacagcctgcccagttggcccctgctccaacctccccttcttgtcctgtcgtgtttcaggaagagatggactgtaaaaagtttccccgaagaactccaacttgtctagctccagaggaacttactctgcatcccttgctccaacgtgtgatgcaacctttttttactcctctctgtctctgtctttctgcttctctgtctctctttctctaatgaagtgattaaaatgtggcaaccgcaaaccccgattagagctgccagaacgttatctttacctttggctgcccgagccgccctttgaagtgcccgcggccgcggtgtaatctttaaccatctcctcctcctcccagagaggaaagggaagtgggctgaaggcggcggggcgggtaaacagcggcgccaacacccaagtggagacttgaagctctctgtcatccctcggggatcgtcccaccccttttccccccaccacagcctccaaagccccaaccgtggagacgaggcagcggctcctttcgtaattctgtctctggaaaatccaggcccggagggtaacaggtggttgggtggatgaggtgacccaggaagaacctgactcttgggaaccgagataacgactctttctccctatgaaggatctggggctcgcttcctttgggcagagagaccaggggtcggggccactcgggaaaaggggcgcttgcgaccacaaggtcatgcgtttagattttaactagaggccctgtggcgagcccaccccaaacgctcgcaatcccagtttcggcttctctgatgtaccttggcaggcgctagttgggatgtttgtgaactcagccgttcctctgtttagttgttctgtggagagaagaaacatgaagaggcttgcacaacgctttacaccagatagtgattctttttctccagacagtgaggaagggaaaaaaattcttttatacttgctttgattttgctttgaaagagagggatgatcctactagattttagtattatagatggcagtccccacctcacctaccagaagactatttgggctgctttcctgatctattcccccactgagttcccctttaatggcacttattacatggtgtctgtgctatttttacagtgatggaaatctgctcccctaagtgatcattctgtaagaattcacatttcctccttctaaacagctgcctgtattacaaagtcagatactcacccctggtctctgtgtttaactgctaaaggcagtgtttcaagaatcatatgttctcccgcatcgtgaatttctgagagcagttgggcagctgcacttcagaacctagaacattcttcctcctcaccctgaagaaattggccatagacaagggaagtggtgcagagcccccagacgcttccctaactattgaaatcatacgcttgttataaaacttgtttaaggatacagaaaataaatgtcaggactcttttgtctctcactcaaaagaacgaacatttgttaggcttctgttactaaaagttggcataaaatattgcttcacatcgtttcccacatatctcaaatttatttaactgtgtcctccatgtactctggaggtctttctgccactgttttctatccatcattttagataaccccttggagttaagataccattaggaagttaattatcctctttttcttgtgttgtttgatgacagcctgtatcctggactaagggaacattttctcagtgtcctataaaatattaaactcagagtgtctatgaaaagtgtcacggtatatgtcaaatacttgcatctgatgattaacgtttcagaaaatatgagtgctaaatatcgaaattagtcctaaagtcgataaccagactgattttcttcatgatgtgtgatttcagattttcgtctccttctagaggacaaaattccagaggccaccgggtgctcttccttggtgggttgaggggtttgaggctttgggagttttgttaggacaacctcagaagctctgtgcgaacattaagggtagatggtgccctctttcggttacacctagaattaaccattgaccatttttccttcacttgtgcactcataacttgaaagagaaatctttatttactcgtattcaaaaaagtaagcatgtgtttcactggttttcttattttaagagataacttccttttgctcttgaaagggaacactaagttacagccctgctccactggggtccagtgataattaagctgtgatgagaaagttgcagtgtgcaaaggctgatgtgggtgaagggccagactactatcttttattgtttgttgtaggttatcaccatttttttgagtcaaaatgttatctgctaatcttcctcccctccccacaaaagaacaagcatttcattttcttcaaacagattgatgatggtaagttccagctgtacacatttggcaaatagatttgtcacaatctgataactggctagtctacctcgtaggtaaagaaagtttccttaatctcttctggaaggctagggctatttatatacagtattataatctttcttccccagaaacaactaaatcttaagccagtccaaacattaactctcgtttcttactgaactgcattctccagtggatctgtaattggaccactgaacatgcgtggtagaaacactggaaattttcaaacttaatcttctctgtctcttccggtgattatgggctctgattttacaacaggggctaataaagctcactcagcagaaaataaggaacacacaaaggttaagacataaaagcagacaataattgttttcaatgtttaatacatctcaacctaccagtagtgatctgtcagaaataaactagttctagcatctaatcttattattgttttaatgcatcacatgtggacatttttgctaaccgagtcttaaaagtaagctccctgtcctacttcgatgtgtctgtcatccagttagtgtggcactgaaatagaaaactaaatcctaatgaacaccctactatagtaaacatacttttctttattgatctagaacagccattttatttttaaacaaaagctgagttttcccttgctgttgcttaaagacaagaatatttcatccacaggcaaatttgatgaaccaaaagaaccaggagagagattttttttttaattttacataattttgttacttttccatatgtaactttgtaacaaaacttaattttacacataattagtaaaactaattttagtaagaaccactcaaaaacctcaatcataactagttaaggacagccctgatgtcatatatgtgtcctaagcatttcagatacagttttaaaattaaagaacatttttagtatcttctttgaaaatcatttaatatctttctacttccgttcaagcaaagaaaactaccttaaataaaggaaaaatttgcactttagagatggttacaaagacttagctgttatgagagtcttcaagtttaggtcagttgcaaaatttacacaatttgctctacaacctactttcaaaattcgtttaaaatataatcagatcaatgacttttctgcattttaatgaagttctttatttttcttcaggaccccggaattttcttagtagtatttttactatgagagcaattactgaaactaattgattgaatttgtttgagtgacttttttttaaagtagtggttcttattcacacaatattttagtgcagatttagaataaatatttcactgaatatgttcaacatgtgcatggcagagtttgtgtgacaatgaatttttacacctaataagcatgattttcaatcagatattcaatcattttctgtctttatgtgttttctctaaagtaacggagttctagctgatcagatcactaatgagttagaagattgtaataaaaaacgacttttatagttaagtaaaattgtattttaataatgaaagcatttgcactgggatgtgctttctaaagttcagaaacccaaaactagtctataatttctcaaagtcaagtttttaactgaactatttgattgtttttaatttccaaaacatgtaactgtcaaaggcatagatataatatccaagtcaacaattaactatcacaaaattgaagacattgaaaaatatttatagtttataactgatatataatatctacttactttagtaagtagatattatattttatatatcttctattgaattcaggcatttaagtcactttatagattttaagcatgtttccaattaaaccaatcatatgccataagacagaaatgaaaaacagtagtgagctctgcattgagcaactttgtgaagtaattcttttggttttgttcagttttttttaatataaatttagaataatgctaaatggcacatttccttgtaaacaccccacaagtggatatttctggtaaattgaattcttcatgagaaggcaaattccttcctatgagattcagtcctgttgcaatgtttgcctttatagagtctcattttgtagtagaaagatgaatcagacgggtgttagagccacaacaaatatattacttaccgaagaaaacaagctgataatttagtccggttgctatttttgatgtcttagtagaatttcatttgttaaatttttacccatgagttccaacctattacagctcttctattcccagatcagtcagcttcaaaccatagttttattcagtcattgtagatgccaactatgtgctcagttacacccatggaactcatgctctatgggaaaaggaaaaattgtataggctttgtggtggaattttttttaaatttttcttaaatcaaaatttaattttgttctgcataggtgacttggtaaggaagcagacttgttcttaaaaactctggcattttctcccacctctaccagttaattttcctgttacacggactgaacacagtagaaatgccttattctaaagcactatgtagaaaaacattggtcatttgatttcaaaaaatgaaataatgttatctagtagtaatgctgacggctaacagggtgttcaaattagtagggtgacttttttttttttttttaattttgctctttaggactgcatgtggaatttcccaggctaggggtcaaatcggagctacagctgccggcctacaccacagccacagcaacacaggatccgagcctcctctgcaacctacgccacatctcagggcaccgccagatccttaacccactgagcgaggccagggattgaacccacttccttatggttactcgttggattagtttccattgtgccataacaggaactcccagcgtgactattttaaaaagatgcttcataaagcaggagtcatttcttatagcctgttctgtgtttaagtctgggattggagaagctgaaatttactttgaaagtatgcaaaggaactgaccctcgtccatgaaaccatcttccttacagtcagatggagtaattctccaggaagtaatgatgggatctctttccttcagggcttatcttccttactagacttagtgagctccaccaggctaggaaatgtgccactttattttctaagtgcccataccagtgcctgatactttagaaatgttcagtaaatatttgtaatgttgaattgaatagaattggctgaaccagctgctgaaatcccagtggctaaaagggctcagagaaagatagatgagaactatttgtctgcaagtaacagaacaacgcaaggctactggtatcccacaattatggcttagccctattagaaacacagttaaattatagttagcttaaatgatgcagtgagttgttattttaacaaacacaccagaatttacatatctaaattactttattataataacaataattataataacaactgtcatttttattgttcttcctgagtgccaggcattttgctacatactttactcaatcatatgactcaccacaactttttgacacaggtactattatcatttctatcttggaagcatattagcttattccaaagatatttccactgctcaaaatttgcagggcctccaggaaattatctttaggcctaatttaccagaaatagtagaaaaaccagccttctaaggtttaattatatctcatagtaattgtaattggtgctgaaatatgtcagcacatttttcttcttaggaacacaatttataaagctctaattccagaaatacttcttcaaggaatcctgggaaatagagtttttggttttcaataaaatgaggataatattatttagcttaaggccagtattttgaatttagtagataccttttcatatatctattttcttattaccatcaaaactgtttttcttgtctccaagtcaatgataaattggggtagtgcccaagtgttataaagagtgatcaactgactaataacttccaagttgaaaataaaacttgctatagttcttttgtggtgcagcaggttaaggatccggggttgtcactgcagcagcttggatcactgctatggctcgggttccatccctggcctgggaacttctacataccatgggtacagccaaaaaacatataagtaaaaataaataaaacatattttcttccatgaccagatctatgtgtcaggtaaagtagctgaattctttggaactatttctcaattcggagagacacaatctctcttaatcgtccgccgattttactttaagtttgcattcgcctttacaactgatgagataagatgaatttgattcagaagggataattatttcaacgttttatctacttatcgatctatctgtaagctgcacatggtttaacacttaagccagggatagacttctgcgtaaattagaaatctgaataagtcagagaaaagtcccctccttcagacaaagaagaacttttcctgaactatccatttagtttcttcagtccttcagttcatttgctgctgtaaacctatgtgttaatcatctattgtattacaaagccaacgtgaaaaaccaatagctgaccagttaattataaagcatattcagggtaaagagcacctcctcaagtaggaactcatgattagaaaatttaggttacactgggacatggtctctaaaagctcttacaacagtacacgccactctagttttctgtcaacattaataatttaaaattattgcagatataaactcctatatataagataaataacaaggtcctactgttatagcacagggaactatattcaacatcttttaataaacaataatggaaaaaaacataaaaaagaacatatacatgtaaaaaaaaatattccgccatccccaattttcaagtgtctcatttgtaatgtatgcacattttttttccatgttcattttattcttcttgccttcagaagaagtttgatttcatcttctaacaaagactgtgatgatcaaggggaaagcaagccatacacacacacacacacacacacacacacacacacacacacacacacacacacacacacacacacacacacacactccaacacaccaataagaaacagtttcctttgtgccacttgctcagtggaaatgatacataacattaacatggcagggctgtggagagccagcccttttaaggggagaaacggaggagctggctgtgaaggcaccttcagttcggttcagaagctgaaatgcagaagacaattgtcctggcaatgggagcccaactccagctgagaacgcgtgcaccgagctgaccttcctcgtcagcctgccagctgagattaagctttcctcaagtggtcccttgttagcccagcctgttccccagtgttgctcatctttcacaactggaagccaagtgtttcaaactgttactcaattttgcagccaaattcctttatttcctccatctccttttctgcctctctttctctctctctctctctctctctctctctctctctctctctctctctctctctctctctctctcacacacacacacacacacacacacacagagtgatggctaatcttacatgcacactttccaaagtctacactaagaggagactctaggaatttttatgtttcactaaaaaactagtaaatatttttgtgtttctctctttttcttgaaaccttgattaaggagaacggggacctctacaaaagatgtctctttgcaaaaataaccttcctttgatcaaaaaagccacaaaagtgtacttctcgggaaagttgaaaaccgcacatgatctcaagtggaaaaaatataaaaaatttgctgaatttttaaccttagtacataacaaagatattattttaaagttggttggttagtgtgggctgtgttagaactttcattgtgttgtgtttgggtttacccaatgttattttaatgagtttattggttcaacacacaaaacacggcgttgcccttgaagttcttaggttataaattttcttgcgaggcacttacttgaaggaactctgttacataatcaattggtctttcaatctatcagcccaaaaataacctttcactttctgtatacatgtacagtgaacattatttggctataaagacccaataaaaatttccatggaaggagccacattccttaatcatctcttgtgtgtatatgttaagccttccaccataaacatttctttcacagtgttgaatgaatctacagaatgagtttgcagtcttctagtgaaaactgaaatgagaaatcttcctctttattttctctcaatacttcctgccctgaagggacccttagcagatatgcctgcatttggctataaacctattctgtaattagtgtagcatccactgtccagtagataaagcctagagaaaaatggcaagagatgtggaaaggataagtggggaagtctgccacaggcagaacagagagactgagacctccactaggcactctgcccaagatggtacccttctcctgtgtctctcctcttttttctccatccctctcttccagacagtatcagaggaggactggaaggatgcgttttctttttttgttgttttttgttttgttttgttttgttttttgccttttctagggccactcccgcggcatatggaggttcccaggctaagggtctaatcggagctgtagccacagcaacgcgggacacaagccgcatctgcaacctacaccacagctcacagcaacgccagatccttaacccactgagcaagggcagggatcaaacccaccacctcatggttcctagtcagattcgttaaccactgcaccatgacgggaacgccaggatgcattttctttatctcagacactctccccctcacctagggctttaatctacaaaatgaagggaagtttggaaagaagggtctttttaaagttgtcaactctaagtgcaccaaaactggagtctttgattttaacttggagcagccacattactatccctgtggtgggagactttacctcactgcaccccccattcctcagcagctgttgttctctggagctcagtgcaacagagtacaggaggaagttcaagatcttttttcattgagtcttcaagctcgggtagcaaaaggaaatcctagttaaaccagattctcccagggtgtaagcctatctcatttgaagatctccaaagttttagcaacatgattaactgacagcaaagatccactgtctgttttccagctgctggctttgcccccataaagtccactgcctcagggtcttgtaacacttgtgagaagagttaggtggatatggtctggtcctgagtgactggctattacatagtaatttgaagccccccttgccttagctgtagaaactcctcagaacacggttaattatataatggaaaaagaaaggctgattttgatagatttatgttaattacagtaaaaccaattcggagcttgagctaatagctagctacatctagagtaagtcctactgttgaactaaagggataatcttttatccacaaattaccagaatcactaagctgtatacctaaggtcacattacacagtgcaaaactgaaatttaatcgtgattgctagagcattaccctaattgcccaaataaacggaattaaaatcacttagatgggttgcagagctctttgctgaatcaaaagcaatgcctacctgtaggatttaaaatagaagaggccttggaaatcacctaatccaacaccttcatttccacagatgatgcatttcatgctcctaaaaggtaaatgacaggtcccaaatcacatagctggcaaagatggaactggaacccaagtctccccaaaagcacagtcgaatgctccttcttgtcaccttgttgctccaatgctaggtgctgaagtcaattctaaaactcccagtaactttttttttaattcagtgaattttattatacaaccatcatcacaacctcattttagaacattttcatccggacatctacttttctagatgttgccaagtaccctgttgcttaggcgtgttttatgttcttcttatctaggtatgtgcctgaatcttaacctgataagatttggtcccctggtagatacagataaagtgtgtctctatcacctaaaaatctgtaaggcagaaagaaacaatcaaatttggtctgaggaatgaataatcacccacatttcagttcctagtcattaaacatgtctttatggtaatttaaatttcacttgggaccttcatttgacacaattcatgctgccttcacgtctcttctgtcttttccctttctcccttagcaccaaaaccgcattaattcaggttctcctccctttttacctggataactgtggcagtttcctatcttgatccgcagagacacctccccgccaagacatctatactgtctgccaccccacaaggttgtccaagatgcagcttggctcctgtctctcttatattcagtcctttggtggtttcctgtggctttcctggtagagtcgaactccttagtttaacccgcagaatcaccttctcttgcctcatcccccacccagaaacccgattgccaattcctgccccctccagagtcccccacccccaccccacgttctcctctctccagccatatcattccacaccctgcagtttccatgtctcgggcctccaggtgttcacctgtgttgatctctgcttggaatgcctctcttctttttcatcttgccaatttctgtgagtctttccatactctgttcaaacattacctcctctggaaagctttctgtgacccacccatcttggatgctcccacagctttctgaacccacctctgtgacagccctcaccactcgcgatcaccatacctgatctccttgtctgttttcactgtcggactctgtacccaccttgaatcgtaggtattcttggaccaaaggaaaaaggagtctccctagattaaaacactgatcactatgtaccaaccatgattttcattttttgtattttatgatgctttgctatcttggggccttgcctacccaggagggactgcctccccagggcggtctcatctctggagatagcagtgactcccctgcaaatagccttttatgtgcagaccaagcaacccaaagcccacacctccaactacttcatttatcaggctcttgcaggaccctgacactccaggtcaatatttcctgccctaatcaccccagagccaggtcccagacaactcagggcaacctacccccagaacctgctgacgtcaatcaaactagcaattctaagtctgcttatcctggcagttccttcccacagaaaccataaaaaaaaggctcttgcctgtgctttctttcctctccttcttcttcgtaactgaccatccatggggtcccacttggccagatgtgcccccctgctcttggcaactgtgaataacaaactgccttttcactggccgtcaccttgtgctctgttggcaattccatacccgaataacatgaaaacctatatcttcgaacaccctgtgcccttgcatgcctgtgcccaggcaacttcaaaagaggtcatggccagggcaccattttctgtctctaggggacattctcttgttcccctaaactctgtgtaagacttgcaccttgtccatgccaaagataaaatgaaccaccctcgagttcccttaggatctggcgttgcccctgctgtggctctggttacagctgtagcacgggtgtgacccctggcccactaacttccacatgccatgggcacagccaaaaaaaaaaggggggggcgccaccgtatgtgtaacatagagtgagatgagttgatataatctctgatataatcttatggtgaccaagaggtcctaccctctaaactcagcaaactgcattgcttataaactcccaggacaggaaaaaaaatcaaaaagtgttttaaccactttcacccctagtttctcatttgattctcatataacaagcagtaaaatagctaaaagcatccctaccttagaaatgagaaatgatgttacagctgctctaacactaaatccaagctcatgaccctatcttaaataaccttagcaggttactctcagttttctcatctataaaatggcaataataatgacatttacctcacaggattgttgtgaaaattggtaaaataatgcataactttgcatatttcctcttatgtggagcatgcattaaattttaggtatcggtcttgttggtgatgatgttacagctgctctaacactaaatccatgcttttcaaatattgtattgcctcttgttgttattccccaaaatagccaaattaattgccttaaaacttcataagtataaatgaaattttcattaaacaaggttttttcatgttaaaaacaaggagaaccagtgcctcctaggtaagagagtaggggaagcttgaaataagtagggcatcacctagcacctgctcaagtaaaaagattttaactttcatctctgatccttggtagcaaacagataataaattctgtgtattgaatgaggttccctattttcacaaatatgcacaaatctccagggttgtggcaatttagtgaacactttgtcacgagctggtggcccaaagttagcagtccatcctcagacatgacttaatgaagggctcaccatttgtggtcctgggcttgatttgtttttatcttctgacttttaatctcacccatccccacaatttttcctgagtatatggggaaaattcaatgtggtaacttcctgggcttttagaagctcccatctcagcggctgacttgcctctagcagcaggcaagagatgatggatttaggggactaagaggagataggaaacccaatctttcttcagaaccaaagatgcactcaggagaaaaacaggttttcaacaaaatgaaaatggatttcttacattgacacctgaaaaaagatttctactctaagggaagaaaaaaaattgatgtaaggaataagcttatgtctgtctatctgaaaatacactcaaaagagttgaaatgtcaggagaatttcagtagctgtccttggtaaagaagcagttggttcagtgcaactgccggaaggatctttgcagtcagtgatgacaatgaggctaaaatgatttcctggggaatcatagataccagtgacttgaatctgtggatgggggaagggttcaaagttaaatagggggtcaaagtaccactcggcttgtatttgagcaggaggcctctggaattaggcacggtgatccccagcagagagggaagatgggtgggtggttattggaccagacagtcttttaactctgctggatgagcctgtgtcaccatctctacagattctcagcacccctctgccaaagaagcaagctgctttacccatgaccaatgaaatcaagagcacaggtctaaaaaagacaagccaaatctcccttctaggaatacttttccatcttactaaatgtggaaagatgatcctggaattctcaagtgcctactgaattactaaagtaggaagctgatttattttgcagcccaaaagccaaaactgaaagagctttcagacagaagttctgtgttctccagcatcacagattgacaaagtggaatctaccagcccagctttgttaagcagtttaaataatcactaaaccaggggggatcagcagacaccgaaaataaagcgtcaaatgaagtgtcggaagggtctgtcttgttggccaagcaaagtcctttaggtggggagaggggtgagagggaagggacagagcagactgtggcaaatagggaaagaattttaaaagagcaagagagactcgaaggtgaggtgaactgcggggctggctcactgaaagccttcaaggatacacatacacacacacacacacacacacacacacacagagaggcatgtctctgcatttacacaatggaaactcatttcagaaacaacctcaaaatctggaaacacaagtatctactttatctacaggaaagtaagaccactggcacactgctaggagctgggacatcatccacattacggtgcagcaaaacaaacacaaaccaaaacactcagaggagcagtgctctagactagaagtctagatccgtggttctcaagcttgcctgcacctcagctccatctgagacctgagagtgaccatctcgaggcccacatcacataccgaagcaatgaaatcagaatctctgggggaagaagccacccatcagaatgatttgttattccccaaggcacattcaacatgcagtcaggtttggaggacccctgaacgaggtccagctaccccactcaccagccacagaacctcggtgagaggcggcacctccctgcctcagtgttcgcatctgtaaaggaaaagaagtgagcacatgctctgagatctctcctaacgcaatcattctatgagtccatcagcttgacatgtgtgggtattgtactagggcaggtgtggcaggatttcagaaaatgggaggtttctacacacccgcagtcaaagccattttgtgcagcaaatctgggagcattgtttactctcctcagtgagcattacttaaaatgactttatttgttttacagtttagagggcctctgtaggtagcctcaggagcccattcatttcattaacattaaatcaacaagcagaccttctttctgcctctctcaagcaggattaatggattttggcatgatttgtgcatctagcagcaaaacctcgtgctgagaaagctagcaggcttgactaattcccagcagtgctaacttcataaacttactttaagcttttaactttgactgaagattaaaggtaatgacttcacgctgttacattcgaatgcatgatactcaagaggcataaaaaaacctatacaacatgacaagctgtttgataaattaacattatatatgtttgcaggtgtgcatatatatatatatatacatataagtatgatttttatgcttatgaaagtattcaattctgtgtatgtaaaaaaggatcttttaaaactctaagattcatgcaataatcattatgccaaatagggtattgacaggtgcttatgtaaagtgtaagcatgagaacctatagttaaaatatcgtatatatgctaatacaaggcccggtgttttgcatagctgtaacatgatggactgttgaatatgaataattttctaagaagcaaaaggattctaccctttgagaccagttatcacctagaaagaatcttccatatgaccatgcttgtccttttcttcttccagacagtcttaaattaaatacaaaattcagagaagtctacataaagaagggagagaaaagggaaaaataccatatttccagcccatctgacaagactacataaaaagcttttgatcaaagggtcttaagtacactttactggtgtgacaaacaacatttttatccctttattagatttattttcttctgaaaactgcagtgaccagggtatggtttgtgaaagaaagctggcagggttgtgttctttgattcctaattaacaccttcagtctcaaaagagaaccaacagaaataccataaaatgttgcagctgcattgtgattccgcttggttggtttgctaaaagtcggctgggcattggcagctgcaataaagagaaagctcaatggatgttttgtattgtgcccactgaagtgaaagaaggcaaccattttaattttcaaatgggcagctgcagagggaggtttaaatggagctaaatacaagatttactgttaatttagagtaaataaacaaagaaaataaaacctaactttcaagtttttacagtcacgtagaaattttagtgagacattgcgaaggacaataattgtggagctttttagctactgattggggaatttggcagcaaggaatcacattcattatgaaaatcaaaaataggctcttggaaagagctgagtaagccaaagctttagaaattgacatttccactttgagaggtaaaggtctacccttcctgtctttggtaatgtaactcccttagccccactttattgaaaccctaaaattcgttcagatcagaattaagatgcaggcactaccaccacccccacctcactttttttaagagcatggagttgaaatttgccttgggtaggggaataaaggaggcatttaactccggtcagataaatctcactcacattcaaaaatgcaattaaggaaatatgtaaatctagatgtttcatcaaatgctagctaaggggtgactagttcttttcgaacacaatgactgaactaaagaactttagtgtctgtgtaaggaccattgtccaagctgcagtccctgttggaaacagagtccggcctggccgtgggaaatacctacatatttgacacaggccctatttctgagagaaaggggatggaagagaaagtatggcgattttactgttgcattctaatcagccatgatctgcctaatttgctttgatcaggaagataaactaatttttctatgggctggataaaagtaattagcgagccaaagctttcatgttatttcaaaagagctaaagtaaagaggatcagaaattgtaatggtgggagctaaatacatcttttcggctcatctgagagcattgttaattcaaaataggatgctgacacagaagagaaactccagctaaagatggtttcatgggctgcactaccattgtaaatcctgggatctataatgctatgtaactaaacagaattacaaagggtgatttataattggcatcggtcatagagccctgttcttttcggggagacctttaccttcatgtgaaaaggtacaattattgcctcgtcctaaaaaacactgttggtttttccaacagctttgcattgaagttgctatatttaaatggttgggatagttggatgtgtttatttgaaccctccatcgctgtgcctctaaagcacacagacacagccagcctgggattctacaatcctggatatactagacatcaaactgaaaacgctaatcaggaacctgactccccagatgcttaagtttcacagcctggttcacctgtttttgtgccagttaccaacttaagtaatgccgcgtcctcgtctgtttgttctgaatacccaaacacatgtagttattagataagaaggaaagcattgagaacataggattttgtttcttaaaaaaaaaaaaaaaaaatccactatttagtgttcctttttttcttgttgatatgacctttgcattacttccttttccaagctgttgtacaagatatggtatctgtaaccctactgtaaacttagttgcttttttggctactaatctcctttgttaaggttcttagataccaaagggtgcagttcatgtcaaactcaatgtgcctaataaaataagtagatcttttaaaaggatctctcagaggttggagcacatcaagtttcaaagttatatagaaactgtcgtagaatcattccaaaggagactaaaaaataagggcactattctactttgtttttaaaattacttaaaatcatttgatttcttgttcctcatgtcttcgccttcatttggacagctattgtccacatacattttaattctcgtatcattgtcctaaacgttcttagtaatagcagtgtttaaaaatgcaaataagtctgttttcactttttggctaaattggtgtaaggtctagaaaagatggtaactagtaggactaagacgtcaatctatggtgtggaggagatcttaataacgtcaacttcatattcctccacagtcattcattcattcacaaaaattaatattcattgagtgccttttctgtgtgctaaatgctaagcgtatggcagtgcccaagatgggcacaatccctacctttgcagaggagacagacattaagaacatgcttatttaaattgtcatatataggatggagaaacaacaatgtcctgtagagaacgatattgagctatattcaatatctgataaaccataatggaaaagaagatgaagaagaacacatatattatttatatatgtatacataaaactgaatcaccttgccatacagcagaaactaacacaacattgtaaggcaactatagttcaatcaaataaaaaattgtttaaaagaaaatgcttatttaaatgtatgttattagggagttcctattgtggcacagcagaaatgaatccgactagtatccatgaggcgggtttgattcctggccttgctcagtgcgtcagggatctggcgttgctgtgagctggggtgtaggtcacagacacggctcagatcccaagttgctgtggctgtggtgaaggccagcagctgcgtctcccattggactcctagcctggggacttccaaatgcctcgggtgcggtcctaaaaaataaataactaaatgtatgttattaagattatgaaagcactctgatacagaatgggagtgcgcaaagagagaaactgcccagtcttggtagtcagagaagacttccattaggaagtgacatttaatctgagacttggaagaccagttggagttggccagaaggagagcaaaggagcattctagacagatgttcctatttcagtctgagcactagctgagctctcttctagcattctcaaactgtaacatcaaatcatgatgccaaggaccttgtactcgagaggtattaatatagttacccctagggggcagggtcctcctctgagatctgacctatcattgctgggaaccctttcctagaaatacatcacaaagatctgtatggcaaagggctttatagttgcattctggtttcataccttctgctttactagcttatagttttgccaggcatcggaattcattttcttattcatctgaaactcaattatgaactcgtttatttgcacctctaataataacctgaaaagaggaggtggtagcatgtatggacctgtatgtttttcctggattcaatgggaagccaagaagtagaaggcaagattttccctctctccttctctcattcacagtaaactcttcaatttttgttcacaagggggagctggtcagacctaagatgctcatgcattcatcctttcagttatccacctaacatgtacagggcacttttggtcaggcaggtcccagactaagcccaaaaaacagagatacattaaggaacagcctctactcgctggagtttgggatttggttgaggaagacggacaggtcaaccaaagcaagagagtagttaagagcctcatgctgcagtcagaggctctttgttggaatcctggcactgccacctattaccctgaacacgtcattcaactcctctgcagcttccatttcctcacctgtaaaacaaaacatttctcctagggtccttcacgaaggttattattgcagagcacttagaacagtgcctggcaggtagtaaatacaatgtaagatcagccatcattattttagagctccatgtaaagtcccgtggaagcaagaaaaggaaaccattcactccacatggagttgaaaatattcctcagagaggatcgtgtttgccctgctcttaaaggatggggaggattttaccacgtggatggggcatgtccttcaaatgcgagatccccatcaacagaggcccaggagtgagaaagcacatggaaggaaattctcccatttgctgggcatgaggcggcagcaactgggcatgagagagctttggagagagagacagaacagaaagcagctgagtggccccaagaactcacaaggagagactgagagttcagcccggaggaacttagtgaagtgcggtttgaatcatgactgccaaatgaagtcccaggtctttattggggtttcccttcagaggactgggccccccacagaacaccaagcctacaattgactcattatatgactcagggttacccttgaccctttctgatctgttttgtcatctgctaaatgagagaatccatccagggtccatgttagtgcaaatatcaccctggattaactttgcgtatggtgcgcgggatgcctgacttgcaaattaatgctcccagccatgttcaatgctcaggtgtttacagggtcatgaacctgtgggaaaacagatgtggaatcagactgggggctttctccacacccacttcttttctcaggagtcgtctggctccagttcttcttcctcccttatgtagcggcttccattccaacctgccttagactccacttccatttccagttgcacaacagggttaattttcatccttctcaatgtcctttctcatccatcaaaataagaatcatgaatgaatggtgtctggttgaacctcaactccgtggtatcttaaggttcaatcccacccacttctgagaaatggccacggtgacctctgctgaaaacaggattggatcatgacagatccacccctgggctgctccatctcatttgggggccataatctcaacccctgcaccttttctggccatgagcactgcagctggaatccacagtttagctttatctagacctccaggcttccagtgacctttgagtccttcccaccccacctccctccctcatgtctcagaaaggacatgtcctgcatctttcccaaagccacccccagcttggatttccctcttctcttctgagacccaagaggtatttatgtagcagagtgtgccacgcaaggtactttgggagtgcataaagataaagacaattatcaggtcactttcttctggaagtttgaaatagaaaaggtaggtattattctacacactgttaaatttggtaaacctggcatgttaccccataaaaggattcccactcaaaggtagggcatcctctgttcctccctctttagtctattgatcatcatgagccccactacctcgcacaatacctgacccctatacgtgttatataaatatttcctgtgggtggcgtgagggatttctccctctgttctgctatttcctcacactgttttcccttctctttcctttccctcaatgccagaattcttgcaagaggaggctgtgttcactgccaccatttcatgaccacccactcactcttttgaaatcagcctgccacccccaccatttaactgaaatcacacttttaaacaccaccacagacctcctactgccaaatccagatggtcttttctcagccctcaatcttcccaaccaccccgctggagtagtctctgcccgccaccccctcctccctccctcagcggagtgagtgcaccacacccccagactccagatgctggaatggacctgggtttggccagagtccaaagctcagtttccttatctatataatggggctaataaagcctattatctgtgtctggcccacacacggtcagcctcccagttgttctcttccttagctctcatctcacacatgcattgctgcagccaaattcatcttcctaaacttcaggtcccttccctgttcagaaagctccagcaacccccagagtatggaaagctccagttccatcacctcgcacatgaggtctcctcagtcagctcaaatcatgcccagctatgctccagcagccacagaaaccctgcaggactgagcagctgaggccagttccctggagtcagattggccgccttctagttgtctttgaatatctctttatctctagtgtcttcattagcaaattgaaacattggatttatttaaagctaaagctcctcttagctccacagttctatgatttttaccttttggatttatttttccacagtttccctgcaagtttcctattcttcaggcaaattggactagaccccattttcctaatgggctccttgctttcctgcctctgtacctcttctcttaccatctctctggctgggctggctgtttttccatctctacgtgtgatctgtatcacacactccaggagtgatgcagtgcacaactgctagatgctaggcagcaacctagagtaattccaactgaatgttctcttagcctcagtcaagagtttatcaccaaactttaacatcaaaaacatttaggttagggataaattaggagtttgggattaacagaaacgttctactatatataaaatagataaacaacaaggacttacttatagcacagggatatcttgtaataaactctaacggaagaaaatctgaaaaagaagatgtgtatataactttgccatatacccgaaactaatacaacattgtaaatcaactatacttcaactaaaaaaaaaaatagaacaaagaaatcgtttaggttagaatcttagctttgtcacttagatgtgtgaccttgtgcaaattactttctctccaaatctcaatttctacatctgtataaagtgacagtagtcactgaagaggaagataaaattagattgagtcaacaaagtactcaagacagcacctgacatgtggaaagcaatcaataaatgtcaacaatgatgatgacaagttaagttcctcttttcaaatctctaattggccaaatagtttcaagaccaatttacacagtactgaaggaaatatcaacacaagttaattgattattcacttgaaaagattagtatctctcttacatttcttattatttctctgtaagttattagagtaaattttagcatctatgaattggtttttgtatatggactttaattgtgtgtataatctccagtgtatcaagtgatcatataaaagtttttgaccttgtctggcttttaagccaaagtgtttggaagcctttgttggcataggtaaaaacaaaacatggtatcagaggttacttgatgactctttgatgtgtgactctttggccagtgtgatagagaagcctctgctagaatatattagaatatagtggacttaaatacttgttttaaaaagattatttatatcatggttttgtcagaatatcattttgctgcacatttacaattgcaatggaatattatttgcattgtgattagcttaatgtctgtttccatgacaaatatctcagacactagaaagacagggactatgtttgcattgtttactgtagaattctgtgagcctagcacagtgcctgctacatagaaagcgccattaaggatttgttgagtgagaggaagaaaggaagatgggagaaagggaagagtgaatctctgtattcccatatggagttgatattacagaaccaggagcagggcacagaataaactttgaactgcatgtaaagtataaaaaagactaaaactactcagccattaaaaggaaagaggtgatggcatttgcagcaacgtggatggacctagaaattatcatgctaagtgaagttagtcagacagtgagacaccaatgtcatatgctatcacttacatgtggaatcttaaaaaaggacacaataaacttctttgcaggacggttactgacgtacagactttgaaaaacttatggtttccaaaggatacaggttgggggtgtggggggatggactgggggtttggagtggaaatgctgtaaaattgggttgtaatgattgttgtacaactataaatgtaataaaataaaattcattaagaaaaaaagaaccaatggcataactcattaaaaaaaaaaaaaaaaagaactaagacaaccccaacccaaatccaccaaagtttatactctaggtatgggcatctgacatactgaggactatgtaccatcttgtatacttaataagtgtttcaggaagatgacaactatacagaacggttaacataaatagggaggcagggctccagactaatccagaaggtggagtgaaacaggtagaaaaatgaataaccaaataaagaaaaatcagaattctatcacataagctacaattatagccctccagtagacagatgagaaatcatcctaggtccatattaaagtgtgttccagcaggctagatggtgtgaggatatgtatgttgtttaagtacattcattatcataactgtcttgatttagtccagtagtgatatttaatttgaaattctttgagcattaaaaatcagtatgagcttttttcaaattttattttataatgatttttattttttccattatagctggtttacaacattctgtcaattttctactgtacatacagcatggtgacccagccacacatacatgagctttttatgttcaggaaacaagagtgtagtttagaaaagaaaaaaatttccggggacgcaagctcagaattagaaattcgagtacgagtacagaacgtttcctggggcatgtcctctggatcacacctgcagggagagacggacgtgacagagacaggattgggcagaggaagaaggtgaacagggatgaggatgtaaccaagtccttagccaagcctttagggagctctaacatgggggtggcccttccaaaggatccctttatacctcacactgaccagttattagatggaagtttctcccatgcagtgcccccagggcgtgaccttgagcaaggaggctcactttgtttatttatttgtttgtttgtttaggatgctcattttaattgactcagctagagccctcgctgtctggaagaatcctgaccagagtggggcagggagcatctataacatcccctgcaactggtcaagaaggatttcgtccagaagagtgaaggaacaggccagattgccctgtttcttgggaaagtaataggagttgacttaccacaatgaggcagtgctactctgagaatggtcagccctcaacaggatcccggaaattatttcacatgttaacgcacctccttctccatttgctttctcttctcttccatagactgttttaaccttctagccagttccttttctgtcttaatcatatgtatatatgtctgcacatttctactgtcccaaagaatgtttatgctctttggcttttctccacctgtcttttaaacaaaactctgtctcctgtgattcctcagagagttacaaaagagcttcaaaatactaccagactagctcaacttcatctcccaaccagctgcataaacttcctctcttcctatatttctgttctcctggtccattgggtaagcctcctgccacttaacacctggcaaaagtatgtttctcggttttttttattagaaacttacctcaaaactcctttttcaggtaacataaactgatgaactgaaaatgagaaaacaagcagtctctggtaatccacagcacagaggttatcaaatggccacttcctaaccactcagaaaaatctaaataaatataagtctgtgaaacaacagcagcaaagacgtttcgttggtcttcaactatgaactcttgaaaggcagtcattgcacctgttctgtgctcctttccattgcacaatgtctagcacacggttagaactcaatagaatgagataattttttatccaacactgcacctgggaagactggcaggctgccaccaactagatggctggtgtacttgagtgattcctggtgtacttgagtgattcctgtgcctttatgtagaagtagaaagtcatgaattacagcacctagacagactgctatgaggattatccattgaatagaacagttttttgtttgtatttctttaatgaggacaaaatatactggtataatccttctttatgaatcaagaggagaattgaagggagaaaggtagattacgaaattatgttacttctgtatcatcagtcccgtaagggcttaacagtccttcattttagcaacgttttttttaactaaacagagttttaaaaattgtgttttcggagttcccgtggtggcacagtgtttaacgactccgactaggaaacatgaggttgcgggttcgatccctggccttgctcaatgggttaaggatccggcattgccatgagctgtggtgtaggccacagatgcagcttggatccagcattgctgtggccctggtgtaggccagcggctacagctctgattggatccctagccctagcctgggaacctccatatgccgcaggagaggccctagaaaaggcaaaaagaccaaaaaaaaaaaaaaattgtgttttctcttcctaagcttgcagagatttaagacttcttgcttgataaagtattagatgatatgggatatcatgcagagaaaaatcatcttataacacctttaaactaaaaaggaaggtgttgaagaaatggaaggtaaatgtgatagattaaaattttaaagcatccatttgctttggggataaaaaaaaaaggtaatacgaaaagatgttctgcatctctgattcttcatgatgatacacttttgcatttgggttacctgccaaaaaacccgaaatagattctacatgtgattaatagtttcctctgtctgtcctggtcttttatgagtactgtctgaggcagatctggcaaattataaggaattcaggaattgaaagcctttcttgggtatctaaagtctggttgtcattttcagattgtccaaatttaaggaaactgaggagttggaacataactatggctcttgagggagctgttattcagtggaattggcagtatgtcctccttggaagtaaggagagtgcaactgatgttaacattgcagtgaatcttgagtgataatgagagttaccaaaatggttatttgaggccgggtctgaatacttggaggatgaggaaggcataaaggaaactaatgtagaccagatttttttaaaacaaagatagccaccacttatgatgtgccagactgcccatatgttatcttacatactccttacatcaaaccagtgagatttgttatttttcttccgtaaagaaattgaggtagttaaaagaattttctgaggtcatcaatttaataaataatggagagaagggacttaaaaagaggcttttctgattccaaagcctatgctttccccaggactccaagaagaatttccagatatgaactttgcttaataatattagcagtgatgacaatttttaacttgctttcagaaggctgaaatcttaatgaacaaaatcttactttaaaaagtggtgaagcttcccgttgtggtgcagtgattaacaaatcagactaggaaccatgaggttgcgggttcggcccctgcccttgctcagtgggttaaggatctggcattgccgtgagctgtggtgtaggttgcagacacagctcggatcccgagttgctgtggccctggtgtaggccggcagcaacagctccaattcaacccctagcctgggaacctccatatgccacaggagtggcccaaagaaagaaaaaataaaaataaaaagtggtgaaggtaattatatctttgcaagggaattgttcaagatttcactggtgtggcagtacctttggaatgaatttggccagaagaaatggtgccataataaacgccagtggactcagtacagaagataccgtacactccccatttgctgtcgtatttaacatctgcttcaggaacggctagttgtcatttcagctgattatcatgatctttcttgggataatcaaagtggttttgtgtgtttgtcacttcattgggctgtttcctaaggagaatgttatctgcatgtaacttcagtataattaataatatcaaaaaaaattggaatgagggcatagatcaaagctgatattttccatgggacctgaaggaacagaataattgacactagtttggcaggttgtgtgaaaatgacacagaaattttcgttgccataattctgattattgtgcgtagaaactacttattctgttcatgtcatttgcacgataatgctgtaagtttagagatgaccctcagtgaatccctaattaggtgttctttaacctcatgaaaattcaaagaagtattttattgtttgaacttgtatagcactactacatccaaagatctcccagagccttatctgaattcttattaattctcatagtatttctataaaattgagatcaatgctttaaaaaattatatagaccaaggaacattatataatagtaatatgtgatcggcagattgattttcactagacgttattaatttttaaaaatgataatcaattattgttcatgttttagccttcatctattttgtttgcttgccagaagtggtgctataaaacaatgaactggtaagtgaagcagtacccaagacaggtacatgcagttggagcaggaccccagtgggaaaagaatataaaacattttctgttttaaatagcaacagttgtaaagttgtcaatctaaggctcctatttgaactgactgcacttgataaaaaaaaataataataacaagctgtgacaaatagaagggcaagatttcagtaattcactaaagaatgtctctcctcaaagctttgtggcaaaaatgtcttgtttggggcacaattgcagagttgaaataacagccttgaataccctggagaaatgactacgtacctaaggtgaatgctgaagccttctgtcttgaatgagccttttaatcaaggcattatctaggctctgaaaaagtcttcagtggtcctacctaaagattaatgggaaaagccgaaaccgagtcggcctatttgcagaaacaattttaaagatttaccgatggttgacaaatgccatttaagctttcaaagccagccatatgcttttgaaaaggatttaaaggaaatcctcccactatagaagacaactttgaaggatctctgtggtggcttttggggttttttaggtatgtggttgtgttgttgagttttctctttcccgcccttttttccacatctttgctgaattcctacatgtggcccagaaaaagacgaccgtaagccttcatctagtatattcctagattcagaagatacctagatgcttttgtttgtcagtgagaattagacaaacaactaattaggcaactgcacatatagagtatctttctcaattacctttctgttaatacagggctgtgactagcaccccagtattgaaagtttagtgcagcatgcttcaaatacacaatttaagctttcaaaaggtgtttagtgtttttttttttaagtttttatgtcaaaacatagattattttagacttctagaatgtcacagatacatacatattataaatcagattttaatgaacctgggcatcattgttcttccacattcattcctctacattttttttttaatttagcttttactttgtattggcgtatatttgatttacaatgttgtgttagtttcaggtgtacagcaaagcgattcagttatgcatgtacacacactccttctttttcagactcttttcccatataggttattatagaatcttctgtgcatcttgccacaggttttttggctcaggtaaaaatgtattcagcctgaagagtaacacatttccaaagaaattatattcaaactgtttctctgcagggtagaaatctccatgcagctttaaagtattgcccagtcacaaagtagatgagaatgggccgcactttatcttttgtcattctttctatacgcacttcagtgatatcttgaggcaatttcagaagcaggacgactcctcctgtcttcttgacgggtctccagggagctggataagtcactttgagccgcactcaggctcccgtcgccctggtgctgccctgggcaccagcaggttcaggagcagcacgctctggccttgtttgcccatttccccgggtcgctgacagtgatctgacagaaagcgacactgacacaaagaagccgagaggggcaggagatgggggtgaggtcaaagcagagaaactgagaaaatccagtgaagtggaagctgaaaggataatcacatgaaggctcagagcgggtgtctgggctcacttcagtcagccactccccaccccatccccaacccaggaagaaatgcttccacttaactgcctgggtagcaattaaaggccagtgctgtgaaggatgggtttggacacatgagcctcaaatccctccttggaacagctggaaagcctagtagtcagcttcttgtgctttcttttaaaagctagagcctaataaaggctgacccataatgaaagcagcaccacagaacacccagacccacaaagagcgcccccttccattcctcttttcttacctggactgttggaatggcctgctgggggctgactgtcctccagttcattttgtaggcggccaccaccagggtaatcctagaaatgtcatttttataccatacatgtttctgtcacaagtccatcaatgacttcccagggtaaccaccccaccaagtatctggtacttggtcacagaatctaaaacatctcatcacatagcagaaaagtgactccctttttatcccctttcaacccctctggcttcatcaatgagaaagtctcagtgaggactaatttgttttcagtgcctctcttacacccatgcaactcccttcttaccaaaggcaagacaggcttcaggctcagtttgacctggcaaatctagctaggaatataactaggatttaataacatcatttggtttttactgtatagactttaacagcatccttctatttatgacaggtgatactggttttctgcttataggaagaaatagaaaattttcacttttactaaacttacctaggtaacaacatatgtctatacacagatcaatatacatctaggtgtgtctacttatatatatgtgtgtatatatatctactatctgtataatatagatatatacagatatataaaatagtattagaaaaagtaaaataatggttcaggtactatccttatattgcaaaataatgcatgagtagtttgggaaacatagagctatgaaataaagccgaatctccttgtatgacctgaaagtcccattataatcttactctcacctgtatttccaaggcaggcttagttttctctgcagcctcacctacacctggtaacaaactgtaataagccatatgtagtagtccccagctcccctgtcgatcatgtaccatacctgaagcctaggttgccttcctctctcttctccactgagataattcctactcatccttcaagactcaacactaagccaccctccaggaagcctttctgccccattgtctcacccaccctgcccatgctccccttggctcaataggtcctttccattgagctcccacagcatcttgtggttacatatttattagaactcttttcagagttccgtggtggcttagtgggttaaggatccagcattgtcactactattgctcaggtcactgctatgatttgggttcaatctgtggcccaggaatttccacaggatgcaggcacagccaaaaaaaaaaaaaaaactgtaaggaaaggaattttgtctcagtgtctgacatacccatctgaaatacagtgtttgtttaatacatgttttatgaattaacagactctttttatctagaaggcaaacaaacagaatatctaataaccaaaatttcaataatagcacagttaacaattggtaaataatgcaccctacttaattggtaagaatatgggctttaccaaagagcagaaacaaccccagtgtccattaacttgtggatggataaatacaatgtggtatatccacacaatggaacattatgcagcaataaacaggagtgaagtagtgatagatgatacagcatgaataaaccttgtgcatgattccattctgtaaaatgtcctgtataggcaagtctatagagacagaaagtaaactagcggttgcctagggttaaggatttgggagggttatggttagtgactgctaatgggtccagaatttcttttctgggtgatgaaaatgttttaaaattagattttggcgatggttacacaactctacgaatatgctaaaactcattaaaatgtgtatttaaatgggagaattgtatggtataggaattgtatctcaataaagttttgttttgttttgtttttaagcgaggcctctaactcagattgccgagctcgtggtaaatcctccacaatttatcagagagtggtcattggcaaggtctttagtttctctgcaactcagtctctcctctgtcaacagaggtagtaacagtgcccaccagaaaagagtgagcatggtggagactgaaaatgcccagcccagtgcctgtgaattagtagcacacagtaaacattagctacctttattactaaaggattacatggtggattctgaatcagggggaaatagaatatattttaacatacctataatattaactgtcttagtcctttcacaaatggtgatacacctatagcatttacagtcttcttggttaataaatcatcttgtatatagtatgaacagtttttaatcaaatgcaggcaatctccacacattatgttgcaatttaatccacaaaacaaccctatgagataacgattatttagcgcattttatggacaaggaaagggatacaaagagaggataacctgcttatcagaggcaaaatagaaggctttcccctgtgcaagtgtgcccacatatacacacctcacaagcagggtaatggcccattgacagaactgcctgaatggatatgacgggctccacaatgctttccttatttgaacacatctcccacatcaaactgccacaacaagccctcctgtcttggctgagatttgacaataagaaatattcttttgaaatggaaaataggcagagcctttatctcaagaatgctgctaacaaatacaacctgagaaaggatgggcctggggggtctgtaccacacaggcgtgatccttgtctctcaacaccagcattatgacactattaagaatgctggctagacaagtgcttataacatctgacttcaatcactgtagtcagtagcaaaaaatgtgttacacatggcatatgcataccgcagcatgtgctgattgtatgaagcacatcctatgatcaaaaaggacaatgcaaaactaatcctaagtggatagcagaattcacttggaacatctgggagcaaactttatgactccgggtaaaatcgctctctctctaagagtattcaatgactccactgggaaacttcagcccacatatactatgttataaataattcatatatgagcataatcatgctgtggaatttaattctcacagaatctctgccttcaaagagattaaccatctagactgaaagattttaacagtgtccatgtctcattggattttgcattcccagcacttttcatgggcctgacaaatctcagtaagtgttagttgagtcaataaatgaatgtatgagtgagtgaattatgacttgggtggggaggtgggggatgacataatgtctttattaattaacatttattcaacactttttaaagtgttccttattaatttgttgggtgatatatgaatgaactttaaaggaattaatacagcaagcaaagtgtctagaaagagactgaaaagtgaataacatttccattaatgtccattatcctgtctaattcatttttaaatgttttttaagatttgatttaaaaagaagagtaaacttggggtgaaaacctaagtcattcttaatttttagagtatcattattcagtttatcagattactggggttactgtgtgaaacaaatgaaattataaaacagttatatgtattttcagacctctaagcaatacactttgtgttcatctgcaaccattaaagagtcagcatcatactggggggggggtgaataacgcctccctttcccagaggtcagtcttctagcaacttcattctcttaaagcagagctaagaaatgtaatacgacgctgtgtgtcaaatattactgtacaaatcccatacactaaagcctcttttaaaaaaggagcctttgactctctttcacatcctatttgttttcttttattcataagcttattaagcttgattatctagcttctgaatgcaagtagagtatcaattgcagttacataaaatctgaacaccaagatgtctttcccatttcattgataactgcaaaatttatccactggtaatccaatcaattgcagaaataattcccaaattagacccctaagaaccctcctatactcctggtgggaatgtaaattggcacagccactatggaaaatagtatggaggctcctcaaaaaattaaaaacagttactgtatgatgcagcagtcacactcttgagcatatgtctgaacaaaactataattcaaaaaattacatgcacccaaattttacccccaaaggaaacaaaccctccatcatttggtgcacagacaaggagtcccacagaccttactcagtagtttcaaaatatgttccagtgcagatactaccattatgatgatgatcatgaatatcaagaaaaatttaggcattcctggtgtggcacagcggaaaccaatcagacaagtatccatgaggatgtaggtttgatccctggcctcgctcagtgggttaaggatcttaaggattgggcattgctgtggctgcggtgtaggctggcagctgcagctctgatttgacctctagcctgggaccttccatgtgctgtgggtgtggccctaaaaaaaagaccaaaaaaaattttttttaattctttttaagaaggcattaaaatatagaataacaacatttttaagatttatatatgaaattttaacatttaaaaaagttaggcattttaaagtataagactcaatttagtgggaaactcagatctatggaatatatattctaaaatagatccagtaaatggggagaaattgttcatttttgcatttaaggtttataaatgaatggcttacttccaaaatcttttaaagtactgcattagtaaaaatattacaaagcaacatatgttaatacactctggattatgtctttgcctgtgttcagttccacatgacagtatgtggaaaaatcaatttacttaacttcaggctttcaaatttccttttagacaatgttaaaactataaacaatgtggcaactgaccatctacaagagtgaagctgatagaaagtgaattatataatgcttgaacttggaaaaggaggaaaaatacatcatagcatagagttactttaaagctttaatgttggccaagggagattaaattgcctttggggtgtacagatgcagaagattccgttggtgtgattttcaaagcaaagacagatgatattaggaaataaagcatgagggcttctgctttaatctgtccacattaatgttttgatctatgactaattggaattgcaagtatggttcaagaatattcttacagccagtctgaggctaacagacatgttaagtcttttaaaaaggcatctaggaagattgcttgaccacaaaagaaagaacgtgtgaagaattgttctaattgaagtctgctagtgtgtctaattcaaaattaatatggaatatgcaccaaaatgacaaaatagtcaatgcttgctatcaagaaatagagaaaataaactaaaaggtattacacaatctccaaaaacaaaacatctgaccctttttaagcctttaaataaaagaaaaagaactaccacccaagagatttattacatccaaagatgatgtcatttctctgcttaaaatcctgcagtggctctcagagcctccagaatagtctgaacttctcaacatgacatatgagccagcggggtctgcttacggcccttgacgctttttcaggccttgtgttgggtcgtgttgtgacacaagcacttccactcaacaacctatcgtcgtttctagagagcagcctgctctgttatgccctgatgcttgtgtctgctgcccctcaccccccagcaacactcttgcttctcacccctccctctatcacagccaacacatctaagactcggtttaagtattgagataatagaaatgtgatgaactgtgtaatgtaagcaaaaagaagattctttaagaattctttggtgaaggaagagatgaattcccaaccagctaatgtctgggaggggtcttatggggagattggacttatattaggttcctaaaactaagaaatttttaccatttttattttacttagaaaatgcagtatatctgtacatctcctgtcctccttggtcattttagtccttccgtgacaccttgagagttagcttggagatgctaataggatagaaaagtcaccagcatgtttatgattgaaacacaggatgcctttatttcggaaagtttttctctctgaggagacagtcagaaaggactcatatattaatgaggtaagaattggacaccgtccagccacccaggtcagtacataaacagcacagttacacaaacccaccagttggaactccacccactccctctgaatgcccaccttaccttactattcttcttttgacacttaaaaaaaaaaaaaaaaaactacagaaacctcatccccttcaagaagctctcccatctaaaggaaattgtgttaacagctccatgggcatgcagtaatgaaagacgttctttagaggcatgcagacttataaatgggatcactctgggtaagttacttgatttgcacccaggctgtctctcctctgggattcaagcatgactgatgaagggtttatttacaaggagaacccaagacccttggaggatggatgtaaaaggaattgtgcccacctgcaacttttggtcaagagcagtggtactggcagaggtggcggcattggtgagggtggtaacgataaagtcctgaatactttttagtcgcacgtgggaaattcctgggttgagcatctgaggcacagttgactgatgattgatagcatctgacacatcaccacagcaagtcagggcagaagagagggtaaacagtacatttgttatagcttacacactgtggtcaagagaactagttcttggcatgcagatgtgtcatcccggccaaagctggaacttccaggatgatttgggaagaaaatctgttggggactaaagtttctcagtgtgactaggagaaaagccttataaataatatattgtggctaattttaatcaagctgttacggatttaaaaagtaaaagattatacctaaaacaagtagatacctgaataattggcatgcggcttcaacttaagaaaaatatagggagttcccattgtggctcagctgtaatgaatccaactagtgtccgtggggttttgggtttgatccctggccttgctcagtgggttgaggattcggagttgccgtgagttgcagtgtaggtagaggatgttgctcagatcccacactcttgcagccatcactgtggtgtaggcggcagctgcagctctaatttgaccccgagcctgggaactaccatatgccgcaggtgtgaccctaaaaaaaaaaaaaaaagaaaaatatcagaaacacatatcagattaagcatgtttctcttaagtgaaagaaaagtaatgtaaaaaccctaaactgacaaaagcaaggagaacaggaggaaaatcatcatagatagtatgtgttaatgaacttgtgaaatgacttagcagagaaacagcatgggcgaggtccagaagcaagccagttcttgcgtaaaatcttagaaaagggctcttttgattgtccaagcattgttgaaaacagtgagattcattgaaaatcttaataataaaaagttaaggagttcccatcgtggctcagtggttaacgaatccgactaggaaccatgaggttgtaggttcgatccctggccttgctcagtggttaaaggatctggcgttgctgtgagctgtggtgtaggttgcagactccgctcggaacctgcgttgctgtgactgtggcgtaggccggtggctacagctccaatcagacctctagcctgggaacctccatatgccagtgggagtggccatagaaaaaggcaaaaagaccaaaaaaaaaaaaagttaaacatctcccttaactcacacaaccagacaactgtgtcctcaccatccaaatttccatcacaaaaaataaccctggtagaagactggaaagttactgtctaaggagagtgagagacttggactaggaagtgtaaaaagttgtttctgagaagtggaaatcagggatggaaggagtggaacggggcctggtggctgcatttttgtttttatatttgttttcatatttgttttcattataaagtttgtagttgtttgaaatatatgtgtgtatacacacacacatatacatatatgtatatattcatgtaatgactctgacctaaaatgaaatttaattgtaatttatttttttatttattatttattatttattttttttattttttgtcttttctgaggctgcacctgaggtatatgaaggttcccaggctaggggtctaattggagctatagctgctggcctacaccacagccacagcaacgcaagagccaagccacatctgcgacctaccacaactcacagcgacgccagatccttaacccactgagcaaggccagggatttaacatgcaacctcatggttcctagtcagatttgttaaccactgagccacgacgggaactcccagttgtaatttaaagaagtaaaataaaaggggggggggagttcccattgtggtgcagcagaaatgaatctgactagcatccatgaggatgtgggttcaatacctggcctcgtttagtgagttaaggatcaagcgttgccacgagctgtggtataggttgcagacacagctgggatcccacgttgctgtggctgtggggtaggccagcagttacagctctgattcgacccctagcctggaaatttccatatgccatgggtgtagccctaaaaagaaaataagaaaaaaaagaaaagaatagaaagtttcttgttcatgaaaacacacttaaaactacatgctattgtattaatgcatacaatccttattatttattccacaggcagccctggggatttttcttaaataatgacctcctttcatgacacagaactgccagggacctagtgggtaggcctgccacacaagctgtgtgtgctcccctccagggcaactggaattggcagtgtgacaactaatggtaactttccatgagtattagagctctactttttactctttaatgtgtagtaagttgtgtaagcttacttcctcatggttggtagggttccttacctatgttgctcttttagctttatcatcaatatgcactcatcgagttcccaggtatgattcagttcgtttgattcattgtaaagatacaagcatggattttacctttttaaggtttctccattttttctcttccatctcttccccgttaataattttacatttgttatagcttacacagacattttgaatttctgattatttatgtagtcagacttgggataaaagactctaaatgttgcactatttctacattttcttaaaggttttaacgcattaaccaataataatgagtgataatgcctttgtttttaaaagttcatccgagatttattggctcttaaggcactcttaagcatataaatggatttttcatagcaattgttaaggaaaggcagacaattggttttaggacagcagtactttggaagataaatagataagtcaaagtattggtggctaaaattaagttttatgttatttctcagattaagtcagcccttggtctttttcctgttgagtgtctttttcaagtctttcttctctaattgttgactctgctccctctactggaatcttggttaaattgaatggaaaaactatctgaaattggagtgtctcttcagagttggtctgtcagacccagattgaaatgagaacaaacatcaagctaggaaattatcaccataacataattgaaaagtgccactgggggagttctcatcgtggctcagcagttaatgaaactgactagcatccatgaggatgcaggttcgatccctggccttgctcagtaggttaaggatctggtgttgctgtgagctgtgatgtaggtcaaagatgtggcttggctcttgcattgctatggctgtggtgtaggccaacagctacaggtcctattggacccctagccttggaacacctatatgctgtgggtgtggccctaaaaagacaaaggaaacaaaaagtgtcaccgggacacagagaaatttgtgtctctcaatctttctaatgcaagtcagtgtttaagattactttagttttaaggaaaggagttctctggtggcacagcaagttaagaactcaggtcgctgtgtcaccgctgtggcttgggtcatagctgtggcacaggtctgatccctggcctgggaacttccatgtgctggggatgtgtccccccaaaaaagaagcattttgtttttgccagatgcttaacccactgagcaaggccagggatcaaacccacaccctcacagacattatgccaggttcttaacccactgagccgcaacgggaactggaatatttaaatcaaattcaggtagtttaattcaattcaagtcttggagaattacatttctatttttactgctttgttttgagttttaaatgcttcaaagcattacaaaaataacaagtgttcattaagtatatgataggcatttcagaatatctcataatatgcttataaatattgcagtcacattccccatatcagtgaggtaagaacagtaacgatttttccatttaatgggagaaaaatttcaatttagagagaagggtatgtgtctatgcaggctcatatactattcaatgaaaaataggaatagaatgtatgcctccctatttgattttgaaatataatcatgtttctacatcttccacattcaaccagaaactaaccaaaattagtgagattccttgggttcctttttcttttcttaatccattgtttgttcatcaatacctttttgctaaatacattagcgtttatagtagagttgtttcttaaagtacactttagccagctgtttaatggaaacacaaggtagacacagaattatttaatctgtttgctctcctccaaggcccacaaaaacatccttagtaaaaataatcctcctagatataagaaaggaagggcacaatgccagagaatgcagccctcatgcctctggtcagtcactcaacaaaagtatatattgattgtcagccatgtgccagacactgtgctaaaagctggtgatttagcagccccctgccctcatggcattcaggaaaggaggacaggaaagtaaataaagatgggaccacagtctattctttagtggaaggaatggtggtaggaggtgatgggagcagggctagttctggagggacaggagtcggggaggcaggaagaagagcaaggcaagagcaaggacctgttctgaattggaaggtccaaaaggcacctctgaaaagtgatatttaagaatgaagagcagcagctttcagaagcgaaaaaatataaataataaataaataaataaataaataaataaatgttcccgtcatgactcagtggaagtgaatctcactagtatccatgaggatgcaggttcgatccctggccttgttcagtgggttaaggatcagatccagcgttgccgtgatctgtggtgtaagttgaaggcatggctcaaatctggcgtttctgtggctgtggcctaggcccgtggctacaactccaaatcaacccctagcctgggaacctccatacaccctgggtgcagccctaaaaagacaaaaaaaaaaaagaagaagaagaagaaagaaaaaagaaaagcagcctttggaatggagggttggatgcagatgagtgtctgcacacagcgagcgcacagtaaagagctgagaaggaaaagcactcagggtctagaagcaaccgcccagggcaggtataacctagatgggctgagtgaggggaccctgagacagatgaggatggagggttaggcaggagccagatcatctggagcttcatcagctgtgctaaagcactaggatttcattgaagaacaaacaggaatccaaggggagggggggcacagatttcaggctgtgtcagggaatcgtctcattctgttttgaaaagccacgtttggctgccatgtggtgcgcctgtagaggggccaggagtagaagcaggatgtggaagggcacttaggaatgaaggatgagtggccacgggcaggcagatgtgactaggagatgacagctgcttcctctggatgtggccttgtgcccggtacttgacaagcagtttctctcatctgcatgactccagcaaagcctgggtgcaggaaaccacaggacaagatcctccaaatcacttcccttgaacagaaagtttattggaaatgtacttcttgagtcaatctaaacggcacaacttcaggacacattgggagaataaaagccagtctggtgtgaatggaagaattgttggacggctttagaggcaggtaaacctcacccagtagatcaggacaggaccatggagatttcaaccaacaactagtaaagccctttcgaggtgccagatgctgagctaaggtgtgcacctccattacctcattcaaccctcatcaaggcagcaggaaataggtgttatagacctattaggaagttcagaccacaaaagcctaaaagtgacatcaagcaagttgggggcagaaccatctcttgagatcctgacctctttccaagcttgtaacccattttgccatcctcataccataaccagagaacaacacaggctccatgttgcaaatgacaggcagccagtaaaggacttttgaacccatgggttgaggcaggatgatggatcagatctgcctttttaaacaataacttagagcctctgcgtgagcttcaggttattagacagaatgtggatgggagagagaggtttcatcctgttgacatggaaggattcccatggggaaaccatgttgactgcagaatgtttccatgtcttacttattgcacagggcagggaaaacaatgggaatttctgcctgggtgaaataaaatagatcttgttcctttaatgaaagctttttgaagaccttctttgaaatattgctttgggggtcttttctgtgtaattattatggaaatattaggtgccccagaattattcaaaagactcagtgacacagcctcagagtgactccttgggatatatacacacaggttcttagaaacttttgctctgctccttgctttatattttcgttgttcaataataaagtaacaaaataatctgcccgaaaggatttgtattatgaaagagtatgagtagggagggaaagtttttctaaattccctctttcaaagaaaaaaagttattggggagttcccgttgtgtcgcagtggttaacgaatccgactaggaaccatgaggttgcgggttcggtccctgcccttgctcagtgggttaacaatccggtgttgccctgagctgtggtgtaggttgcagacgcggctcggatcccgcttgctatggctctggcataggccggtggctacagctccgattcaacccctagcctgggaacctccatatgccgagggagcggcccaagaaatagcaaaaagacaaaaacaaaaacaaaaaaaaaaaaagaaaaaagttattggaaccttactttttctgtcatcacaaaaggcaaaattaatagttttttcctgtgtgtaattcacatatacaacttcagtgactctcaaccaagggcagtttgcctctaggggacatttctttggccatgtctgtgcattcattttccagggttgccataacaacgtaccacagactgggtggcttaaacagtagcaatttattttcttacaattctggaggccagccgtccaaggtcaaggtgtcagcagggttggtttctcctgaggcctctctctttggcttgttgatggtcatcttctccctgtgccttcacgtggtcttccctctgtacctgtctaagttcaaacttactcttcgtataaagacaccagtcatatcccatcctaatgacctctttttaacgtaattacctctctaaagaccctgtctccaaatacaatcaccttcagagttactgcgggtcaggacttcaaaataaaaattggggggaagaggagaatgtaattgagcccataacaatcaagttcttggttgtcacaactaggagaggggtgctcctgggcattagtgggtagagccaggggtgctattaacacccctgcagtgcacaggacagccccctgcaatgaagtattatcaagccccaaatgtccatagtgtcaaagttgagaaactctgttttagttctaaaaccagttgcttggagttcccgtcgtgactcagtggttaacgaatccgactaggaaccatgaggttgtggttcggtccctgcccttgctcagtgggttaaggatctggtgttgccgtgagctgtggtgtaggttgcagacgtggctcggatcctacgttgctgtggccctggtgtaggccggcagttacagctctgattagacccctagcctgggaatctccatatgccacaggagtggccccagaaaaggcaaaaagacaaaaaacaaaaacaaaaacaaaacagttgcttgattttctttaagcataaagaaaaacaatatattcctcaagtgaacattagtgttgtgaattatggtataatcttcagatgctgcaaatatccctgttcaaatacctcatacagagacagatttgtgggcatgaaagagctttgtaaactgtaaagttctagacaaataaatggagttgttgttaaaagaaaaaaaggaagcacaatatattagcttctttatccttcaggaaaaaaaattaagatagttccagatggactgggatggggagaagaatgaaggcagagagggccattaagatcccattttggagttcctgtcatggcgcagtggaaacacatctgactaggaaccatgaggttgcaggttctattcctggcctgactcagtgggtcaaggatgcggcattgtcgtggactatggtgtaggtcactgacgcagcatggatctggcattgctgtggctgtggcataggccagcagctgtagctccgattagacccctagcctgggaacccccatatgccacaggtggggccctaaaaagacaaaagacattaaaaaaaaaaaaaaaagatcccattgcaatggcttcacctcccctcccagataagcccagggttctgactggaattctggaatctggggatgggaaagagatggaccaagacccaatcattctactcctagacgtgtacccagcagatatgcacatatatatatacactcaacaaaaggcatatatcagaacgtttatagcagcatcattcataataaccacaaactgaaaagaatccaaatgttcatcagtagttcagtggaggagttcccattgtggctcagtgttttaagaaaccaacacagtgaccttgaggatgtgagtttgatccctgacctcactcagtgggttaaggatccagtgcttccacaagatgcggcataggctgcaaaggtagctcagatctagtactgctatggctatggcgtaggccagcagctacagctccaattcgacccctaacctgggaacttctgcatgccacaggtatggacataaaaataaaaaagaaagaaaaagaaagaaaaaaaaaacagttccatggtaaattgtagtaaaacaatagatgagatactacattgcattgagaagtaattaacaattgctacatgcaacacatggataaatctcagaaatgagtgaaaaaagctaggaaaacacaaaaagctatgctcactactactaagtgaaagaagccaatctgaaaaggctacatactgtatgattctaattatatggcattctggaaaatgcaaaattatggagacaagaaaaaattcagtgattgtgggggcaagaggtagaggggagaatagccagggcacagaggattcttagagcagtgaaaatactctttatgatgttgtattggtggatccatgtccttatacatttgtccaaacccataaaatgtacaacaccaagggtgaaccctaatgtaaactccagactttgggtgattatggtgtgtcagtcggttcatcagttgtaacagatgtaccactctggctggggatggtggtagtagggggggctatacatgtgtaaaggggagaggggtatatagaaagtctatgtacattcccctcgattttgctctgaacctaaaaatgctctaaaaaataataaataaatatatatatatatataaaatattttagaaaaaaactatgcaccgtatgattccctttacagaaagaccaaaatcaagccaaactagtataaagtgttagaagtcagcagaccggttacttttggggggagggaagtaatgagtagaaggaaacatgaaggaacttccgaggtggcggcagtgatttttgctggtttacaaggtgtgtgttcacattgtagaaattcagtgggctggacatttaggatttattcattttcccatatatgttatgcttcagtaaaaagtatttttcaggggttcccatcgtggctcagtggttaacgaatccgactagaaaccatgaggttgagggttcgatccctggccttgcccagtgggttaaggctccggtgttgccatgagctgtggtgtaggttgcagactcggctcagatcccgcgttgctgtggctctggtataggcctgtggctacagctccaattagacccctagcctgggaaccttcatatgccgcaggtgtagccctagaaaaaggcaaaaaaaaagaaaaaagaaaaaaaaaagtatttttcaaagataactttatttggagttcccatggtggctcagcaataacaaactcaactagtatccatgaggacgagggttcaattcctggccccactcagcaggttaaagatccagtgttgccatgagctgtagagtagggctcagatgtggctcggatcctgcattgtggctgtagcttcagttcaacccctagcctgggaacctccatatgggtgggtgaaacctaaaaagacaaaaacaaaacaaaacaaaaaaaaaaccagaatccttcgagaatgatgccttggaaactgtagtgtctagaacttccagaaagagagagtctagttcaggagaaaggtccagtaaatcatatgttcattagatttgacaacatggaaatcgctatttctatcacaggagcagtgtccctggaatatagagatggaagcctgaatgtgtgggttgcaaagggattaggaagtggagacttcatgtggagactagaaatgacccagaagagaacgagacacagtgggtagcaggtaaaggaagacatgagatggaaatagctgggtttttgtttgctttagctgtggaaaatatctaactatgtatccttatgaaagggaccatacataaccatgtttctttatgaaagggaagagaaagaagttgaaaatacaggaaattgaagaaattgttgatgaaataatgtgctgtagcatcccggggttggaggacccatgagcaagagaaaagcatgggtatagtccgccgggaacaaaagagagcaaaaatgtggcccatttcatgtttggtctaaagaatcccagttatttacacgaagtcagagggaaggtgcccacgatgggagtggctgtcgattaagaggtttgaggcaagtggtaaaggttggcaactgtccctgtgggaagaaattgagccataacaaataacaggattgtgcagcggtgttgagagcccacaaagaccgtgaatttatagcaactaatctgcacatttgtgagttttcctttgctagtagcactcagtacgctgagatcataacagagaagatgaatggtggtgttctccctgagctggggctctgccagtagatgaaacaaaagacagtggagggtgatggtgaggtagtggtccaggagctcaatgtgtgatcttaggctaagacaagcctttgattccggaaatatcaaaggactagaggttgaaatagaggacccctaagctggacagagacgagattgtgtctgacacacaggggtctgttaagcacaggagccgtcatttggaaatcggataatgtgttctagggaaagagaatcacatgtgtaaagacagatgcaaagattctcagtgtaactggaacatatgttgcacagaggaaagtggcataagatctaggttgaaatgtgctttaggccagatttgtgaggagcctaagaaggggtggaccttggagttcctgttgtggcccagcagttaatgaacctgactggtatccatgaggatgcaggttccatccctggcttcgctaagtgggttaaggatccagcgttgccatgagctgtggtatatataggtcacagacgcagcagcttggatccttcgttgctatggctgtgatgtaggctggcagctacggctccaattagatccctagcctaggaacttccatatgccacaagtgtggcaccccctgctgaaaaaaaagaaggggtgggccttggttcctttaaaaggtctggagcagcagaaggtctcaggtgggggatcccaagccagtatggttgtgcaggtcatgcccagcacagggtctctccacgaggctgagctccaagccaagctctctttgccaagccactcaccccattcggtggccgcttctgtgggaggaaaatcatctttaatttaccaccctctcctcccagagaagtgcttccccagagcatctgcctttcttattggctagtagtggctctgagtgaagtgacattttggttggacttttcagtgtgacctccttggccagggtgtaggagatgacctagaatgagaaggcacagaaggcaggagagcagaccagttatcagtttaacaaggacctggcaaaaggtcgcagccaaaaaggaagaagccagatgcagaagcaagctttgtagcagaattaactctggacgccttaagggagtgccctggtgactcagtgggttacacatccagcgttgtcactgcagaggctcggatagctgctgggttggatccctggcctgggaacttgcccatgtcatgagcatagccaaaacaaaacaaaacaaaacaaacactgggtgccttagggatgtagggataataggaaagggaaaagttgaaaatgactcagactattctccaatgtgttcctggacctgcccgcaactagtgctcaattaatgcttgtcaaatcaagagctcccattatagctcagcaggttaagaacccaacatagtgactgtgaggattcgggtttgattcctggcctcactcagtgggttaaggatccggcgttgctgtgagctacagcgtaggtcacaaatgtggctcagatctggtgttgccacagccatggtgtagctctgagctgcagctccaatttgacccctagccggggaacttccacgtgctgcaggtgaggccctaaaaggaaaaagaaaaaaaaaaaaattgtcaaatgaagaaaacatagtagagttgcaagcaaaaaaaaaaaaaaaaaaaattttttcaagagaaagatacaatctgcttaaatggaactctctacctggaagaaacagtaccttcaaccatgttaagcctcctaaattttgagtgagttgtagagctcagtgcactcatgtgggaatttagacagagcttctctcatgcagtgttctcagccttggctgaacattcaaataacttgggaagctttttaaaaatcccagtgcccgtggagttcctgtgatggctgagcggaaatgaatctgactagtatccatgaggatgcaggttcgatcgatccctggcctctctcattgggttaaggatccagtgttgccatgaggttgcagatgccgctcagatctgacattactgtgactgtgacctaggccaacggtgaggacccttagcctgggaacatccatattttagcagtgtggccctaaaaagacaaaggaaaaaagaaatcccagtgccggtactggaccactgattcaggaactctgggggtgggatcaatattttaggcatcaatatttttttaagctctttggtgattccagtatatagtcaactcaaggaattttgcaaatcttgttctggatttctagatgtggtccttaacacagcatagatgagagctgggtcagagacagggcagaacatatttgactgtcttatcacaatgaatactccattctgaggaaatctttttgcctagtggccagctcttcaaaattaattccaacatcttggcaaaagatgaaaaaaatgaaagaaatccgttacattgcttcactgatgttaggaaactgaaccttgagccaaatgtggtacaaaatagggactttttaaaaataattgcttaaaaagtaaataatacaaggatttggaaattaagaccccttcattcgctgctaagctcctatctgttccaaggtcagagagaccaaatactataggggaaaaaaaaagaaaagtttaaaaataatttaagagttaagaatgaggatattttggtaagaagtggaaagtttattggaaaaaaggaaaagcaaaaaaaaaaacctagggcaaatagagatctaggctccagcccaggggctttcccactaaattctgcgctgcgccttggagaccactcagctccgtgaacctcattttcttccttgataggtggtgagtggcagtcctctctagcagccagacattctcattctatcatgtttaactcaaagttgaacaggctaaatataatttaagtcttgctcttctagacacaaggcaagaatagcactgctaaaaaagtggtctgagtacccataaccagccaaggttttttttcaccaatccacaacaggtaagggtttgagagtaaaggttttcacagtaattttcacagtaattgaataatttcttctgaatcacataaaatactgaggcttatattttgttttgtttttttttaatgatttttattttttccattatagttgttctgtcaattttctgtattctgtcaattttctactgcacagcaaagtgacccagtctcacacacacacacgcacatattctttttctcaccttatcctccataatgttccatcacaagcacagggaattatatctagtcatttgtgatggaacatcatggaggcttgtattttgtataactttgtgttgttttttgtgttgccttttttttttttttttggtctttttgccttttctagggccactcctgcagcatatggaggttcccaggctaggggtccaattggagctgtagccgccagcctacaccagagccacagccactcaggatccaagccgtgtctgcgacctacaccacagctcacagcaactctggatccttaacccactgagcgaggccagggactgaacccgaaacctcatggttcctagtcagattaatttccactgcaccaccgtgggaactccatagctttgtgtttttatttcacttttctgataacaactttcattatattttagaaagatgtaagtcctcagcaaaaaagaactttttttcttggtcttttaccatggatagtttgggaagcactagcaaaaggtacacatagaaaaggtaaatacagacaagctaattttgacactgcccactttgaatttatattttaaaggctgatttcatttaatttgagattgttattaaaagttattttctgctataggtttatctgcattgttagcagctttttaaaagaaaacagcccagaattcccattgtagctcagcagtaatgaacccatgaggattcctatttgatccctggcctctctcagtgggttaaggatccagcattgccatgagctgtttcagctcggatctggcgttgctgttgcagtgccgtaggccagcagctgcagctcgaatttgacccctagcctgggaagttccttatgccgagggtatggccctaaaaacacacaaaggaagggagcgagggagagagggagggagggagagaggaagggcctgtattagaaacaagtggtaaaggattggtttagcaatagagttgtcccttggtgtccgcagggaattggtcccaggagcccccgcagataccagaatctgaggatgcccaagccccttctatgaaatggtgtaatttggtggaaacagtcagctctccagatgcgaggatgtgaaacccacaggtaccgagggcagacgacttcaggagctctttttctgagaggaaccacaatgtcaggttaacaggagaagttattaactcctgctgggaaagggaggtattctccttttcaagaagcaaattaagtcactccctcctgattggaaacagctaatcacagggtgtggaactaggaatgtggagacaccctcgtttaaatatgaggaggcccacacagggaggttaatttcccccagataatgcagctagcgaatgggaaggccaggacaaacccaagatgctgaactccaacaatacctgcacactttctgtcctaccatgaaactctgcagggggaaccctgacagcaaatgcttaaacacgtttcacttccaggtactgtatacagggtaataaataataaaataatgtggagtgcttatgaattttccttaatatgtaaatttccttgccccatttagagctttctgcacatagacaggatcaatgtttttgcatgtcacaccgggaaatatgagaggaaaggaatgtcagcgccatccacaccagctgtcttgagactggtttcatccaagttatcaataaacatgctcccaaagcaaaccagatccttttaggaaaaaaatgggatatttatttagaaataagtgaaagagttcccactgtggcacagcgagagtgaatctgactaggaaccaagaggttgtggactgatccctgaccttgctcagtggattaagggtccggtgttgctgtgagctgtggtgtaggtctcgcagacgtggcttggatctggcattgctgtggctgtggcgtaggccggcggctacagctcccattagacccctagcctgggaacctccatatgccctaaaaagcaaaaaataaaaaataaaaataaatgaatatccctgagaaatatcaagtctttgctgaggttatgtagtcactgtaaagcatgcacacctgttcggtgaacgaaacagccttgctacttcagggaaatgtctgcagcaggaagtgaaattgctaatcagttatttggctctcttctctaaacaatgccagggaaaaatcagattttttttttttttttttttttgtatttttgccttttctagggccgctcccacggcatatggaggttcccaggctaagggtccaatcggagctgtagccaccggcctatgccagagccacagcaatgcgggatccaagccgagtctgcaacctacaccacagctcacagcaacgccggatccttaacccactgagcaaggccagggatcgaacccgaaacctcacggttcctagttggattggttaaccactgagccacgacgggaactccaaaatcagattttttttttttttttttggctttttgtcttttttgttgttgttgttgttgttattgttgctatttcttgggccgctcccgcggcatatggaggttgccaggctaggggttgaatcggagctgtagccacgggcctacgccagagccacagcaacgcgggatccgagccgcgtctgcaacctacaccacagctcacggcaacgccggatcgttaacccactgagcaagggcagggaccgaacctgcaacctcatggttcctagtcggattcattaaccactgcgccacgacggacactccttaaatcagatttttagtatacagacaatacaaaggttttttttagtttatgtttttggcattgtttagttttagcttgagaacatagcatgttacccagctatgtcattgttcactaacatactaaaatgaataattctgggggtttgccatactctaaaacatttagcagggattttatattccctgtacatactagcaactttttaaaggcacagagtgaatggtccctcacttcttggcatcactgattgaaagttaatatttgataaaagcaccaagctatcaatactgaaatctgctggctagatgcagtggatgtgtccacttgagtaatgggaattattcgggtttttaaaaagctcctgggtgaaatgggcataggtggtcaaaagatacaaatttccggtaataagagaaataaatcctggggatgtaatgtatggcatagtgatgtagttacaatactgtatcgtatatttgaaagctgctaagagtacatctaaaaaattttcatcacaagaaaaaaaaattttaactgtggtaatcatttcacaatatgtacatatatcattatgttgtacacctaaaactaatacaatgttacatgtcaattaaatcaatttttttttctttttgtctttttagggccgcacccgaggcatatggaggttcccaggctaggggtcaaatcggagctgtagctgccagcctacgccagggccacagcaactcggatccgagccgagtctgcaacccacaccacagctcacggcaaagccagatccttaacccactgagcaaggccagggatcaaacccgcaacatcatggttcctagttgagattcgtttccgctgcaccacgatgagaactcccaattaaatcaattttatttttaatttttaaaaatttttatagtgatttttattttttccattatagctgatttacagtgttctgtgaattttctactgcacagcaaggtgacccagtcacacatacatgtatacattctttttcctcacattatcatgctccatcacaagtgactagatatagttcccagtgctaaacggcaggatctcattgcttatccattccaaaggcaatagtttgcatttattaagcccaaattcccagtccctcctactccctcctcctccctcttggcaacctcaagtctgttctctatgtccatgattttcttttctgtggaaaggttcatttgtgccatatattagatttcagatataagtgagatcatatggtattggtctttctctttctgacgtacttcacttagtatgagagtctctgatttcatccatgttgctgcaaatggcattattttgttgttttttatggctgagttgtattccattgtgtattccacatcttctgaacccagtcgtctgtcaatggacatttaagttgtttccacgtcttggcttttgtgaatagtgctgcagtgaacaaaataatacataaaaatggggttcccctattggctcagcagtaaccaacccatgaggatgcgggtttgatctctggcctcacttagtgggttaaggatccaacgttgctgtgaactgcgatgtaggtcacagatgtggctcggatccagtgttgctgtggctgtggtgtaggctggcagctgcagctctgattcgacccctagcctgggaatttccatatgccgtgggtgcagccttaaaaaaagacaataataataattatacataaaaagaagctccttgtcttatctcaagatacatttttttgtgtgtgtctttttagggccacaccagcagtatatggaagttcccaggctacgtgacaaatcagagctgcagctgccagcctaggccgtagccacagcaacacggaatccaagccgcatctgagacctgcaccacagctcacaacactgccagatctctaacccactgagtgaggtcagggatgaaacctgcatcctcatgggtactagttgggttcgttaccagtgagccacgccaggaactccttcaagaaacattttaagtatgaaggccaagtctgagattataatgacatcaggactgaatcttcaggtttgagcaagtcattttttaagtcaaccttcagctgactgttaacctgaagcctcagctggtctcaagtagactctctagcagttggtcttttggcctctgagatacggatggcagataccagcgatgctgtggcgttatgtgggagatagtaaaggaacagggaagtaaaaacagaataaaatctgattacacggagtttctcttaaaacaaaaattgattgagttctgcccaaagtggcttgttgtttcctgtgccaggagctactgtgcagcccagcaggaagtgtgccccagagcaggatcacattacacccttgttttatggttcatttcacttatttaatgcaatctgagaacctgcaaaaggaacatgtttgaaggacggaataggctttttgtcttgttttgttttgttttgttttcagtttctttgaatcaaaacacttgtttttacaaggtacttgtagtcccagagaaacagggcttccagagtggcatggtgaagacatttctctttgtcacaacatttagagcaggacccgacgttgagttatgtctggagttgagttatgtctgttccctttgctgtcctgctcaggtcattttaaacatgaagaattacaaccatggccatgaatggtctaaatcactttgaattccatataagcgcatcccagggtcccttatttgattgaaagcccatgatgaagggctggggggaggtgtgggtagaagttggaggaagggaatatttaagatgcacaagagcgattcatgtgtcaagcagaagaatgagttccttgggctgtactgcttgaaaatgtgtgaattgaacagctggatttccttgtggtggcaaacgcccctgctcacacgctgcctcttttcctctccagggacctcagtatgaacaacatcagtcagctgcccccgaaccctctgcacagtctccgcttcctagaggagttgtaagtatccccggagtcgtgatgcatgcagtcaacgctggatacattcttgggtttgtctctcaggcttactgtgggaaccagataaaacaaggcaaggaagctgtcagcaccaacagttttggcacatgaggaaacacttagttgggaggggaaaagaaagaagggcttatgattctggaaatcaacttatgaaagagtttaccgtgaggctacaactttcgaaggtgagactgagccaaacaaattaaaattttaaaacaaagcctgtttaagagagctggatggctctttggaaatagttaagcagcaggatattaaaagtttgaatactggctcatgtttattaaaatagcaagtaaagctataatcaaaaccaaaaacggccgctcagttcagcttgtgacagggagaattgagtccacatttcatagcaccaaaacttcttgtcctaatcacttttaattactattttcctacttttaaatttagcttccctgaaggctggaacagaaaaacatgaaatccacagaggactgaaaactggtctctgctgaactccctgtaaaaaaaaaaaaaaaaaaaaaaaaaaattcaattcaattaaaaaaaaaaaaaggaggaagctaaaattagctaatgatacacagaggaatcttgattaagtctggtaatcacttcccagaagatgaaaaagaactctgtaaaccaaaatctgcatttaaaaattcatgtgatcctcttacccttttgaggatggggaccaggggagggacacaccatcattgtcctcaagaagaaagtttggcaccatcaactacataaatcctctgactgttgattaaaggacttgtttcagcaatgacctcttttgaaaatacttcctttatgcttcctagagtcagggtccaatttggggaataatttaaattgttgatgcaccacacaatccaccttgggaggtggaaaaaaaaaagatccatcacaaccaaattatactacaccgatctttttttttttttttcaagaggataatattgtcagatctctcccctttagagttggatctttatttatttttgatggccttacattttctaaaaatgctattattttactaccgagatcagctgaatatgcccagattgttaccgaggggtatagtggagaaacctagattactggattactacagcaagtttttatcattctgaaaaaaatggcttttcactctggcttatccaggctcctcttttgcctcttctattttctctctgcccatttcttcttttgctttctttttttttttttttttttttacagccacatctgaggcatattcaagttcccaggccaggttgaattggagctgcagctgcaggcctataccacaaccacagaaacaccagatccgagctgcatctgtgggctctgccacagcttgcagcaaagctggatccttaacccactgagcaagaccaaagattgaagccgaatcctcactgacacgatgtcacgttcttaacccacggaaccacaatgggaactcctctctagccatttctgttgctaaaaaattaagcttcattcagtgcggggaccatgtctgtcttagtcatggatggtatgtaatgaacaatcaatacatttttgatgaatgaatgaaaaacaaatgagaaagcagaacatgtaaaaggagcttcttatacttgttcaccaccacaggaagaagtagatttggctggcattcgccaatatttttaatgattactttgagcaaatttcaaacatcagtgttttttttttcattatttacattcttcttatctgccctcccccaccctaattaatatatagtacttagccctgtttactcccaaaaatcattcccaatgcttttctctttaatgtctttataatacgtgatagcgaatggcttccattcttcctcgacactcctttagactacaaatcaaatcaacaaataattcacttctccaaagactttaatagagcccttctcttggtgacccaacttattccgaataccttctcttccttccttcatgaagtgaatgttaattctcatccaagaccagttttccgaaggatcgagggaaaacgaaacacaggagaaacagggggtgggaactatcattttaaaatgctagttttctaaattgtcttatttttatgtttctggaaactccatcacgtaccttgactacagaatcatagaatttcagaaaatagaggggacctttaacgactatttgttccactttctcattctgttctgaaattctggctgacttgctgaatgtaaaagtgagaccaactctcccttatataattctgtgctgcagctaactgatgttttgaagacatcattattcatttaatggcaaaaggggatagtatttatagaacctggaaaaatgatcagctctgcttgttctattcttttcttcccagggctaaatttagggcattgtggttttaaatacacacacacccatttagtggagcattttgactatgtctttatgtgctgattaaatctattatacatagctttttttttttttttttttttttttttgcttcttaggaccgcacttgcggcatatggaggttcttaggctaggggttcaatcagagttacagctgcccaccaacaccacagccacagcaacgcaggatccgagctgtgtctttgacctatgccacagctcatggcaacaccaaagccttaacccactgagcgaggccagggatcaaacccacagcctcatggttcctagctggattcatttccactgagccacaaagggaactcctagacctagctatgtttaagtctacactttcctgagtacaagatcttcaaaataataatataattataatgataataatagcttactctatgacaggctctgttgaaaatattttacacacattatcccactgaatcctcttaacaacctcataaaggtaggtattattatcattcccagtttaagatttttttttaaaaaaagtacagagaaaacaaggaaggtaccaaggaaatggagttcataagtgtcagaactgattcaaactcactttcaaagacatactcagaaaggacaaggaggcttaggaattgtgcatgattcccctgtgcttttcttttccccgatcgtacctagtacaggggtctgcatggcggaacactttaaaccgttgttagactgagttttgtagtgataggtttgtttatctgtctgtgactgggtatttgtaacggaatgaaaaagagaagtgggacagaaagcaagaacgagctgtctgcccagcagagggagattggatattcaagttatgaattcactgccatcatatttgggctctgagaaaagtatcatcagaattttcagatggagaatttcaccagtttctcattgctcaacaggaataaaaccttagttaatgttttttttacatgtacatacataactgaagcaggatgttttgtacagtagtctacttgaactaataaatgatcatctatacagctagatagagactttatttatttatgggaagacatctttcaaatcagattggaagttctagccttttttttttttttttttttttttttttgtctttttaggactgtacccactgtatatatggaggttcccaggctaggagacaaatcagagctaaagccaccagcccacgccacagccacagacctacaccacagctcagggaaacgccagatccttaacccactgagcgaggccggggatggaacctgtgtcctcatggatactacttagatttgtttctgctgagccacgactggaactccgatatagacttttaggtatctgtcattttgtgtgtgtgtgtgtatcttttgttatgacaaattaaattaagctaaatgttaaagtaagctgttctgaaagtattctttcatttagtttattcagcttatttccaagctttattctttatccattctttaggaagctactacttataatctcaataacattaataaaactcccaacacaagactcactttttaaaaattagtcttaacaaagccaagttaccattcttccccttaggtctaattgcccaatatgtcatttccctagtttaatcacctgtgttataaactgtgtagacaatcatgattcatgtctcatccatcatggatgccatatttcaaaatcctctaagaatcaataaagccctagaggtaccactgacattttttaaatatattgtaaacctggagaaagtgactatgtgaagcaaatatttaacaggaattgaagccataacggttaatggtctccttctcttcctcctgaaattctggaatgcttactgcctctgtcatatatcttagtacatgcaagtgttgtttgtcattcagtatttatgttttatgactcttgcaaggtgacccagcatgcacaagccaaacaccctgctggtgcaatgtgagagcacaaagagagagatctttttcctttgatgggggaaagaaaggaagagaaagaggaaggaaggaaagaaggaaagaaggaaggaaggaaggaagagaaagaaagaaagaaagaaagaaagaaagaaagaaagaaagatagaaagaaagaaagaaagaaagggaatgaaaagagaggggggagggagagagggagggaggaaggaaggaagatggatgaaggaaggaaggagaaaggaacggaagaagaaaggaaggaaagaaggaaggaagaaaagaaagcatctctaagtgtggttaggctatactctgtcgaggtagaagcaaagcccagaattcctgtcctggctctgccacataatagctgataatcaacttgacctccctgggcctcaggagatgatgaagagagctgcctgtgcctgtgttttgagatgctgagcatcaaatgaaatgatgatgaaaagaactttggtaaactatcatttccacgtggagttgtagtaatgaaatgaaaacactttcaagaagattttcacttctttctttcctatttcatgaggttgattttgcagtaatcccaatcccaaggacccaggatttgcgattgtgaaggaaaagctgtttccaactatgagctttaatgggtgtaggcaaccaaagcagatttggtagaaattttgacatcctgaccaatcgttgcatttttctccaacagtatctctacccgttctatgtactcagttctagggacttcacgttaatgtttgctatttggacaaataaaagattctatatttggtccatcagagcatggttcagatgttagaagcaaatggcagatcgtgcctaatgctgacatgtagaagttccttgaaacttagattggtagcttgattaaaccggagggaaaaatgaattgttaccatttaaacagtctaagctaaaggcctttgtttccaagtagtattttaaaatcttcccagattctaatgctattggaatcaaatactcgatttaggaaggaaacctaagaagctctagggaggtctgtttttaagttactattgttccttggaactatttgctcgtattatggttggccttaaagaacatctatctaaatagtagatatctactatttcagatgaaattagtaatgagaaataccaccagacttcctgaaagcacaatctatgcttgttacttctgcttcctcatcatgtttctgcaaatagtgagagctgtcctgtctgtcttttgagatttttgtctgaggattaaatgaaatgatgatgaagcaactttggtgagtggtaattcacctattggattgtgttagtaaaataaaaacaccttcttgcccatgaataatcttctcgacccttgacctggtcagagtgctcagtctctcctcttcatagatctggcaatggattaataaatgatgcttctttacctttaaaaaatgtataaatttttgaaggaataaatttaaaatataggaaatgaaaagaaaaaaataactactgcctctttaaaactttattttatggcagttgatttggtattcttccagatgtctattcatacagctggatataaacatttagatatctgtccttatatatgtccatatgtatgtacatacatcttttgtattggtaattagggcactgttctttcttgactttttacattttttattttttagataatttttgaaagtcacactccatttacagtcattataaagtactggctatagtcttcaattgtaccatatgcatccttgcagcctatcttactcccaacaatttgtacctcccacctccccccagtaaccactattttgttctctatatctgtgaatctgcttctttctttgttatattcactagtttggggttggggtgctattctgaaagtgcctgacctctctgactcctcctctttttctccttgggcttcttttctcctccacccccaactctggtaaaatacagatatgccctaggtttctgcactgatttttgatgcctcctttctctgttctcttcaattcattcgttcaacatgtatttattgaaccaataggaacataaagaatatgacttgtggagttcccgtcgtggcgcagtggttaatgaatccgactaggaacgatgaggttgcaggttccatccctgcccttgctcagtgggttaaggatccggtgttgccgtgagctgtggtgtaggttgcagatggctcggatcccgagttgctgtggctctgatgtaggccggtggctgcggctccgattcgacccctagcctgggaatctccatatgccacggaagtggccctagaaaaggcaaaaaagacggaaaaaaaaaaaaaaaaaaaaacagaatatgacttgctacatttcattcagtcagtcagtcaacaagttcttgcatcagaggagcctacaatctagagggggagacaggtgataagcaaggaaacaggcacataggatatttgctgattgtgacaagagtgataaagagcacaaagagagttaagtaggaataaattacaaaggatgacaaggagggaaaatgtctctaagaaggtgacgtctgaactgagatcaggaggatgagggggacttcgtggtccgaagagctggagtagagtgtttctagtaggagaacagtgtgagcagaggccctgaagtaggaaagaatgtgatgtgtttgtggatcttaaagaagatcagtgtctccagagcagagtgagagggagggaaagtgacaggggatagataggttggagaggaaggcagaagccagatcattcaagaccaatcaggctatgtagagatttcaagtaaaagcaatgggaagccagtggaggattgtatgcagctagtgaaataaatagaatttatgatttataaagagctctctgatttctgtatgcaaaatagattggggaattaccgaagcagcgagacctgttaaaaggctataacaaactcattaggagacatctgcaattgtctgagaaagaaacccaagatttgaactaccacctctgctatatggatgacccctaaatcagtatttctagtgttcagatctcttttgtctttgatctctaggccagtggttctcaaaatgcggtccctggaccagcagcattgtaaccacatgtgagttgtgagaaatatgtgccccaacccaacttagtgaatcagaaactctgagctcagaagagttaacaagctctccagtgggcatctatctggagacaaacataattcaaaaagatacatgcaccccaatatgcattgtggcattacttacaatacccaagacatggaaccaacctaggtgcccattgatagaggataaaaaagatggggtatatacaatgaaatattactcagccataaaaagataggaactaatgccatttgcggcaatttgaaaagacctataaattatcatacaggtgaagttagtcagagaaagacaaatattataggatatcacttatatatagaatctaatttttaaatgatacaaaagaacttatttacaaaacaaaacaatatttccaggtgattctttttttttataatgatttttattttctccttcatagatggtttacgatgttctgtcaattttctactgttcagcaaggtgatccagtcacatatacatgtatacattcttttcttttttctcacattatcatgctccatcataagtgactagatatagttcccactgctatacagcaggatctcattgcctatcctctccaaaggcaataggttgcatctattaactccaaatccccaatccatcccactccctccacctcccccttggcaaccacaagtctattctccaagcccatgagtttcttttctgtgggaaggtgtatttgtgccatatattagattccagatataagtgatgtcatatggtatttgtctttctctttctgacttacttcactcagtgtgagagtctctggttccatccacgttgctgcaaatggcattattttgttgttttttatggctgagttgtattccattgtgtatatataccatatcttcttaagccattcatctgtggatggacatttaggttgtttccatgtcttggctcaggtgattgtgatacatggtaaagtttaagtacagagctactctaggccagtggttcttgaattttagtgacatcacaccttgctgggccctacctcagggtttctttagcagattggaggtggaagggaggatgtcaagaatttgcattgatatcaaattctccagtgatactgacactgtgaatctggggacctcactttgagaatcgtccaccacctaagtgatctccatctaccccacccatactttaaaacccaagtgcagaatcagattccttctcgcacacacacaatactctccacaacaaactttttctcctgcctttccttctcaccatcagctttcttatcatctgaacttgtgatcactgggtctatcactgcttttgcatctgtcattctttttcacctcctgtatcagccaatttccaggtccgaaagctcataattacacattgtttttcctatttgttcactctgtttcatctccacctacttcaggttcttgctatctcctctggataatggttcaaattagttaactaatcacctctcctgattctcccaataattaacaaaggaatcattgtgtccagagatagagaccatgagggtctctaggtgatcttatcaatatgaaaccatgaggattatttcagagttttccaccttgaagaacatctctgtttggaggatgagcattaaaagaggggctaggagttcccattgtggctcagcaggctaagaacccaactcagtgtccgtgaggatgcgggtttgatccctggcctcactcagtgggttaaggattcaacgttgctgcaagctgcagatgcagcttggatctggtgttgccgtggctgtgttgtaggctcagagctgcagctccaattcgaccctcagcctgggaatttttatatgctgcagatgcagttgtaaaaagaaaaactaaaattaaaaattaaaataagaggctcaggagttcccgtcatggcacagtggttaacgaatctgactaggaaccatgaggttgcaggttctatccctggccttgctcagtgggttaacgatccggcgttgccgtgagctgtggtgtaggttgcagactcggctcggatcccgcattgctgtggctctggcgtaggccggtggctacagctccgattcgacccctagcctgggaacctccatatgctgtgggagtggcccaaagaaatagcaaaaagacaaaaaaaaaaaaaaaaaagaggctcggagttcccgtggtggctcagcggttaatgaatctgactagcatccgtgaggacacgggttccacccctggcctcactcagtgggttaaggatctggcattgccatgggctgtggtgtaggtcatggatgcagctcagatcccacgttgctgtggctctggtgtaggccagcagctacagctccaattggaccctagcctgggaacctccatgtgccgtgggtgcggccctcaaaagataaaaaagaccaaaaaaaaagagagagcgagaagctagcaagaagaaagacaaccagccatagtcatgagagcagaaagatttcagggagggatctggaaggcagtgggaggactttgaataagaagcagtgaacaggagttcctgggtggctcagtggtttaaggatctgtattgtcactgtgactctggttactgccatggcatgggttcaatccctgacccaggaactttccacatgccagaggcatggccaaaaaataaataataattttttaaaaagaaagaaagaagcagtaagcaacagtttcttatggccaatacctcaagtgatacagtcatttatgtcctttgctcctgttttagatttccaagctccagaccagcctacttttttggggtttttttgtttgtttttttttgtttgttttggctgcacccatggcaggcaaaagttcccaggccagagataaaacccacaccacagcagtaaccagagtcacagcagtgacaaccctggatccttaacccactgagccaccagggaactcccagaacaacctacttttaaccaaagtaatgtttaaaaactcacatgttggagccataatgctatgatttaaccaaattctcttgagtatacttttctttgtaagcatgagcaaataacctctctcaggcccaatttccttgtaaaaggggaatgatcgtgtcacttgtttgatgtgatttttaaaatagttaaatagcacagtgtctataaagtacttaacattgtaccttgaacttataaattgctttacaaaaggtatgtcctatttgtgtgcccccaaacactgtgtgtggcgtagtgaaagtgcccaaataaattgctaggtttaaattgatgcctaacaaagaaaaaatattcttcagctgttttgggtctgttatgtaatggctagccagccaccttgcagtagttgtgccagacctgaagcaactctagcacgactgccaactgtctttgaaaacaagagcaaatgatccatcaataaatattgattcagctccctgctctgggaaggcagtaagattcttgaccttaggagcctatagtcaaattaggtagactacatctaaaatggtaaaaaatgaacctttatagacagcattgtatagactatatcctacaggaaagtttactgtagactgaggtaattaaggaacacttcacaaaagagttgaaacttgaacttcattagcgtgtggttaaagaagccatagggccatataaccactaaaggatctccctgtttgcctgaagggtttacttattcagtccagtggtcttaggccttaaaccttgaaatctgatgggtgttgatcttatgtaataatgaaaacaaaatggacagttcataaatacacagtttaagaaggagtgcttttaaattggcacaacattgtggatcaactctaatttaaaaaaaagggggggggttggggctgcatttgaccaattagatataattaacttattggggaagggagagtggacaccacagattttgaaaatcagaatttcttgggacatattaggaaagacatttttttctccaatgggaagaaaaacagttggtggattgtcataatccctatatctaaggctgcggaatagacttaagcaaaccttaccaaaactgaatgccagagggttcacctctaagtatctgggccgttccaacgagcatagacatcttataaaacaaaacctgtcatggtgggcttagtcagtcacaatgatgatgcattactgccagcagaggcaagattaagagattatattggactatgagtttccttctgcttgtatttaactcttcaaggacccagtctcttggctcttgtatgcaggaacctaaagccaaacattagtcagttagtataattgaattgaattataaatagtcaatctgaatggtctactgtatttgttctcatcactttgtcttctgaaagtcgctcattacaatattagtatgtcttatgctaactttattttaggagaatttttatttcaagtgggaatagtttgacacttcagatattttatgtttaagtgatagtttaactacagttattcaggtggattaaaactcaggaaagcaaaaagatggagttcccactgtggctcagcaagttaagaacccagtatagtctctgtgaggaggcaagtttgatccctggcctcgctcagtgggttaaggatcctgcgttgctgtgcctgtggtgttggccagtagctgtagctctgattcgacccctaggctgggaacttgcatatgctgcaggtgcagccataaaaaggaaaaaagaaaagtgaaaaggaattcagaaataggaacagagccactgaaattaatattggaaaccacagtgaaggaaaaaagtaccaaaaaactggaaataggcagaacttcagaatgggaaaactactaccattaaattaagcctgattttaatcctcaccaaatttataaaataaattaatgtagtatatatagaatgtatgccttctagatctcacatactttgtaaaagttgggaagtaaagcagtcatttctagaaaccagcatgaaattgcttagtctgaaatgtcttcttttaaatcataagaattgtcatattcacattcattttgcttattatgattatttaagtagcactgctattatataacttccctgctccgtaagaaaggattaggttacatttaaatgaaagcaggaaagagaatgtttttcattttacataaaacctttctgaactttggtgtctttaacttcaaaatgtggaccacgataatctctgatacacctgctaatataaagtctgggaaagtgattttaaaatgttaataatatgtaaaaaaatcgtacaatgaacaagaagaagaagaaaggagtggagggaggataaggtagaaaagaatgaagataagcaagaaatgccatttgagggcaaaaaaaagccttccgtggaagaagcctatcatttgttttagggagggaaataaggtccagagaagtgtggaatctcacagccaagttaatggtgaagaaaaagaaagccaaaaaggaaagaaaaaaaggaaaacaataacaacaacaactaagagttaaaagtcaatcaggttcttcaaaggcatgacctgggcagcctggtagaacttgccaggccttctcacctacctctgaaagtgctgctcagccaaaggcacccatggtggaccctggtttcctgcacagtaattttcccaagagtcctcatcagaggacagggtcgaacaccccttccattctcagtgctcaccccattatattcacacacatctctgtccagagaggagaaatcaaggtccatatctgattctgccattttacaaaatgaagctacccaaaaaacaaacagaaagacaaattttaaaagtttagattcctttatagattcaaattaccatagagattcaaagaaacatacattctcaaaaataccttagttggctgttacagcacctaaatatatttatttgatttatttgaagaggaaagtgtctactgtaacttatctttttttttttttttttttttttttttttttgctttttctggcagttaagtcggtaaaactagcaatggtaggaaaatgtgaatcttgatacccagaggtatcaaggctaaaaacacttttatatcacgtaaagagatataaaagcttttcttattaattgtatcttattgtagaaatgaagctaatgaaagagatatttccatgaagcaattttagaaacagggtgaaaataaaacctcccagccaaatggtatatagctctcgagggttggaacatcattgcccagagatttgttatttcagatcttcacaggacagatttgtgtgcagttcttattatttgtgtactcctttaaaaatcttttttcctatagtttatatgcatttaatttttaaattgctaacaccatcccacatggtaaagaaatgaggggaaggatgcagtgatttccttaaattattgtttttgacaatgagataaatcctccaccagcccagcaaatagcccatcctagattcctttgttttaagacaaactcccacaggccagtgattccacagagtagttctgaatgcagacagagaggctctgaaagtccttgcccagaaaaaagaaaaacaattcatgtgaaatgagctctgtttcaggaaatgtaacagaaaggcatttaccctaacgataatttaaactaatacaataaataaacaaaacgtttttactttctccttttcatttccaaagtcaatgactcagaaaccatcaagctacttgataccctagcagcagtaagagtagataagccccctcgttcaggctctgttctagaaaatactgttgatgtagttcccgtcgaatccaactagtacccatgaggatgcgggtttgatccctggctttgctcagtggatcagggatccggtgttgccatgagctgtgatgtaggtcacagatgccacttggatcccacgtggctgtggctgtggcagaggccggcagctgtagctccaattcgacccctagcctgggaacctccatatgctgcgggtgcagccctaaaaagcaaacaaacaaacaaacaaaattagaacgtactgttgaagccatgactgtgaacaggatggttaaggacattttggctgaaataatagccagttatcctggtcagtttagtctgaccagtgactgtggtggtcagccacttgtgattgtcacccaagcctgagtctgtcatgattcaacaatcaactcttacagcacagtcatacacaattcatgtccttccatgcaaatacagtatttaggataaatgcttgtgggttgtatagaatataaccataaatatgaaatgttgctcctttttagccacccatgttcatcttgaccttagtggtttgccaggtgggattttttttcctcgtccaccaaatgccaccatttacacccccaccaccagtgcatcacaccagcagctcccatttgaactgttctagcctcctcctgtgcttccatttttgcccccaaagtctatccccatcagaaggacccttttgaaacacagaactgatcacatcactccctggctcgaagtccccattgtggccttcagagccctgcttggaatagccctgaactgctctgccatctcacctgctgtcactctcccttggtcattccattccagccctgccggccttcttgcagtttctggaccacaccaggcacactcacttctcaggccttttgttctctttgcgtaggttcctccatggctcagtcctctgctgaaacatcctctcagcagagaggatttccccatcatcttacctgtaataatggcccatcacctttacaaccaatgcccatggatccttctctatcttcttgcccagcttttgtatttaagaaaaaaaaaaaaaaatagggaattctctgggggcctagcagctaaggattcagtgttgttactactgtggtgtgttgtgggtttgctccctggaactgtgaacttctgcatgctgctggctcagccccccaaaaaaaattttaaggagagttttatatgtatagggtaagctattaaaggatttattgtaaaataggtaatttattatattccttcttatgtccttctttcgccagctttagaataaagatttacagaaattttttgcagaatgtaacaaaacaaggttctttaagctgcttttggcatttgtggtctgaacataatcacgtcgcgtttttaataactaaatgaattccacatgaagagagctaagcttacactctctctcctcttccttgttacccacagacgtcttgccggaaacgctttgacatacattcccaagggagcatttgccggcctttacagtctcaaagttctgtaagtaaaccgagtattatttgttatatttccgattttagtgtcaagtggatgctaatgaccttgtaaagattgagttcctctacctaactatttagagccacagggtaattcatccaaattagggtctgccttcattgataccacagaaccatgttatacctggcaggatgtgtatgaaaaaaaccctcatctctaactttcatatcaaaaatgtgtttgcctctaaataaccaacttatcaactcaaacttgtgaaagagaagttccctttccttttgtgttatgaagttggggagggtttctcggtcattcggagtcaagtgaataaagcactattatgctgacacaatgagatcacagcttcaaaccatttgcaaaggtcgtttccttataatgcgctgaataaggatcagctaaaaaaatcaagtttatggtccacttgtgaatagatgtgaaaaagtacatagtacatagtacaatgagtagttatttaaatcactgagacttgtaaaaacagtgggctctcttacgacttaatgtccaacttcctaattttttatcagcaaactcatcgttatggatacctccttcttctagtactcattgtttaatgttctttcggtttatcctgtgcattaaaaaaaggaccttcatagagaaaatagatagcacaagtttaaagttgtaccaggaaagtcctatctcagagaaaataacctggttttacttcctttctttgagataaaagttataaactctacaaagatatttgatatggttctgcttaatggaaactgcgtttcactgacctgagagatgcgtaggctatttttaaaagcaattttatctaaatttaatattttgttaaaagttaaattcaaaagcagaactgtcgtttggtcttacttaggggattgagtttggcgcctccaccttgagttatttttactactaggtagaaatattttcaagttgtcacattgccaaatcctaatgttaattcatcatgtggtaaaattctgagctgtagcaccaattctaatgtgcctgtatttccatgaatccagcaaaatatttggaaccattcaaaatgcatcatgtaaatccagtttagatcagacaaataagatgatgtttttcctgtgtcaagctaggtaccactaagaattctgggtacttgttaaataaattgtattgaaatcaattattataattcacttaacaaaatgtacattggttctagttcagttttgtatactgaactacggagtgtgtgtgtgcgtgcgtgcatgcatatgtgcacgcatcactcaaaatgacattgaacaaattattgctggaaatacaaagatcaataattatggccatagccctgaagagcttataatctagtgatagatggtggaatcaggatagtgtaaccaatggagttccctggtgggctagaggttaaaaatttagcattatcactgctgtggcacaggttcaatccctgaccagaaacttccacatgccatggggcatggctatggaaaaaaaaaaaaagatagtgtgaccaagacagatgagcaaatcacactgatactgtggaacttcagaagttgggaaagactctgtccagtctgatggttttgattcaacttcctgggagatgtgttttcccaaagagatgacctaggagccaaaccttgaagcacaactcagaactaaccatccaccctccacataagttgactgagttaggggacagtatgcatagagacagaggcaggaggagcctggggtgaacaagcagccctgagcatgtggtcctggagccctggggagaagatgcagagtgtctggagaagagcctggagagagagaaaggatcttggtcatagggatcctgtatgtcatgtcacacaaacacacacaaaaaaacatggccttattaggttcttggcatgataagaagccaaggaagagatgtaagcaagggaatggcactcttagactttgcacttcagctagatcactaccactgtgagggggattcaagaggagcaagatccctgccaggaactgtattccctctcccctacattttgccgacttaagactagtattcacgccatttgcagcaacatgggtggacctagagattatcagactgagtgaagtatgtcagacagagaaagacaaatatcatgtgatatcacttatatgtagaatctgataaaaatgatacaaaagaacttattttaaaaaacagaaataaactcacagacttcaaaaccaatcttatggttaccataggtgaaactgttgagaggaattgggagggtgggaatgacatatacacactactgtatgaaataaatgactcatgaaaacctactgtatagcacaggaaagtctactcaatagtttgtaattacctctatggaaaaaaaagaagaatggatatatgtataactgattcactaaatttttaaaaaatcaggtaacatttaaaaagactagtattcagggagcaaattcaattgtcaaatatttggacaattgtcccatatttagtattagagcaatgcttcccaaccatgatgactatgcctgccacagagtgttttggaaatttgtgacagcatctttgtttttccagatgactggtacatagtaaattctcctgtgatgtgctgctcagcccagcataagatccctacatccctcttgacttctgactctctgtccacaccaaacatcaatcagtattagcaaacaaaggtttaacgagagttctgcataagcctaataatcagtacagaaaaaaatggaataaatatactctcttaaaatcattgaataattgaagttcccatcatggctcagcggaaatgaatctgactagtatccatgaggacgcaggtttgatccctggtctctcaatggattaaggatttggcgttgccatgagctgtggcgtaggtcgaagacatggctcagatcccacattgctgtggctgtggtgtaggccagcagctgtagctctgattcaacccctagcctgggaacttccatatgccacaggtgtggccctaaaagatttttttaaaaaaatcattgaataattaacagagtttatgcatcatgtgatttgaagccacatatttagttgcacatgttagtgcagtggaagacttagctcaaaaaacacaaaccaaattaaaaatctgtaactctgaagatgtaaaacgtcttctaaaaaataactctcggagttcctgttgtggcttagcgggttaagaacccggctagtatccatgaggatgtgggttcgctccctggccttgctcagtgggtgaaggatctggcattgctgcaaggtcacagatgtggctcggatccagtgttgagctgtggcttaggctggcggctacagctccaatttgacccctatcccaggactctccatatgccacaggggcagccctaagaagactaaaaataaaaaataaaagccagttttatttttttaaataaatacataataactatcattaaagtagcagaggaacataaacataggactctcgctgaatttcactgacaaagggtgctttataaatatcttgtattatggccttttaaaatttggagagcgcaatcatacaaagtcatacagaaacaatttatgtactggaggtggcctgttgtttttttttgtttttgtttttcactttttaaagctttattgaagtacagttgatttacaaggttgtgataaatttctgcagtgcaacagttattcagttatatgtgcacacacatcaactattttccagattctttttccatatagataatcacagaatattgagtagagttccctgtgctctacaggaggtctccaatggataaccattccatatacctctgtgtgcatatgccagtcccaaaccccagtccatcttactcttcctctatctgacaggatcttccacccgactttggtgaaggcagtatcataactatttctttttctaatataacctcaattccctattccctgcagtgggatatgccctctggggcctcttgaagtatggggggccagggaggtggggagagacaaatgctcacttcagcatgtgatagaactttgagatgggttggatatgctccatggtatttcattcctccgtaaaagagagaattattttgatatcactcctatctgtaaggaacactcatttgtgagaatctccaggctcatttgtgagaatgtaacactttctcgttttctttactacgatgtctataaggtcttggcagcttacctttatcttaataaagattacttccttcttgcccataaaatataaaataaatataatctgttatatgtcttagctgtagatctttttctactctcgatctagacctctagtctcgtttccctgttcttgtacagaataacatctgattgtgtcatttttctcacacccagcttgattcattaaaagtgggggctggagttcccgtcgtggcttagtggaaacgaatctgactagcatccacaaggacaaaggttcgatccctggcctcgctcagtgggttaaagatccggtgtggccgtgagctatggtataggtcgcaggtgcggctcagatccctagttactctgactgtagtgtaggccagcggctacagctctgattcagcccatagcctgggaacctccatatcccgcagctgtggccctaaaaaaagaccaaaaaaaaaaaaaaaaaaaaacaaagtggagacaaacatctgactcattcatataaatccgtcctaggatagttacatctgagcatcacatatttaatgatacatatatcactttttatcagctacttttatttctcctttataatagagttagggcactactttatgttgatttttttccttgaaatgatatgtgtagataggctatatttttagatgaattttatctaaatgtaatggatgaggcattacaaaatgttgggcattaaagggaaaattgagatcagtagggttaagaaccaaagtgctagaagtgaaatgctgaacaaaacagtcctggccctaccagcatggaaactgtctcaaatgttctacaaagtccaaaagtcatcagaagaaaggaaaaaaatgactctattgtagcatggtgaggaagacaatatacaggctaatctggggccccaatcaatataaggagggtaaaaggggcatcaggcaaaagaagagaaagataggtgttcccgtcgtggcgcagcagaaacgaatccgactaggaaccatgaggttgcgggttcgatccctgcccttgctcagtgggttaacgatcctgcatggctgtggctgtggctgtggcgtaggcctgtggctacagctccgattcaacccctagcctgggaacctccatatggcgcaggagcggcccaagaaatagcaaaaagaccaaaaaaaaaggaagagaaagatgatagtaatccagttcaagaagatgggaaatgaagatggcaatagccctccttgatgaggactgttttgtgtctaaaatcttgtttctgtgagctctggaagatcactaaaatttaagatgattggagttcccattgtggctcagtggatcaaggacctgacattgtctctgtgaggatatgggtttaatccttggccttgctcagtgggttaaggatgccctgttgctgtggttgtagcacacatcatatctgcagctccgatttgaccccaagctcagaaacttccatatgctgcaggtatgactataaaaagaaaaaaaattttaagatgattatccttatagatattgactttcataaaaaataagttatttgttggatttcccattgtggtgcagtggaaacgaatctgactaggaactatgaggttgcaggtttgatcactggccttgctcagtgggttaaggatcctgtgttgccatgagctgtggtgtaggtcgcagatgtggctcagatctggcattgctcttgctctggtgtaggccagcagctatagctccgattagacccctagcctgggaacctccatatactgcaggtgtggccctaaaaatacgaaaagcaaaaaaaaaaaaaaaagttattggtttggtattgcaaaatacccaatgctgtggaattctgaaccaagctattctcaacaaaggttcattttcttgggtgggcacatccatcaaggaatggcttatcctcctgtcactcagcaccagaaccaaagccacatggaggtgatggataaaaaccgtaggaaagtcacagttagccagatttctctcattcccagaagagaacctggagagctacttgatatctaaacataggacagataggcagaaggaatgtgtaaggatcaagccatcaatggttacaaagtcccctttcaacctgtaatttcctctctgcctgctgcactgtacctgatcttgtttagttctattggccaaaaaaggtaaaaattttagaaaataaccaaaacattgttttgttaggggtagcatggggacgcttgtgtgaaacagtatttcagtgctcctcaaggaaagaaattaagaaaatatgaagtcatcatttgtctctagagaaagtgtttcattttcttttcttaactacatgtgaatgataaaaatgaagtggtaagtgcctaaacccatattcagttcatggtacattctgcttagagtccatgcactgaataaaggattatttacaaagaaattggcagccatgttatgaatactctgttttaattgcacgctgtagtgctataaaccctttaattgttttgtctcctgctggaatatcttaaacaaaaacagacttttctaattaactcactggagacttataaggctataatggagataatttgctcttgttttgtcattggaaagtaattaaacaaaagttctttatttagggagacatcagaacctaagtgaattttttaatatttttggtgttttactcgtagaagtggttgaaaattaaattgttataaggtagctcaggggtagctttaaaagtgttcattagtaaagcttgtttaagttccattttctgaaattagccactcaaagctactgagaaagtttgcaactattcatgaagaaaaaccatttaaatatctcttatcctctattccagttttccaatatcttaggaagactacattaaaagagtttcactatttggttagcaaagcataggaggacagttcaatacaaattttgagccagtaatatattacccaaaagtttagacatcaattaagtccgttatctgtatcttatttaaatttatcacacaattaataatttggaaaacatttttgccaagttaaaattatatattaaaactgcttggaaaggtataaaataacatagaggtgtaaacttttaacgcttaggtaaattttgcaaactatggaattcgagctacaattttcctaatttcctaactggtaattgactgtgagatctccctaaaacttgacttcattcataaggaaacaatatattttgagacttttgatttataaaatgttaattgaatccaagtttaacataatgaaattacttgtgtaaaactattggattacaagtgacagaattatattccttaggaaatagaatctcagccgtggtgagaaaagcataattttttttttttttttttttttgcttttttagggctgcacccatggcatatggagtttcccaggttaggggtctaaacagagctatagctgccagcctacaccacagccacagcaatgcaagatatgagccgagtctgtgacctacaccacagctcacggcaacgtcagatccttaatgcactgagcaaagccagggagcaaacccgcaaactcgtagttactagtcaggttcatttccactgcaccacaacgggaactccaagcgtaaattttatgatctccagatattttcatctcttatttatatatgagcccacactagcgtatatgaaaacataataaatgctttcatattctaatcctcaaacatcttatcaatcaaacgatattattctactcccattacacagataaaagaccaagggtcaaggagattgcttggcttgcttgccaagactcaaaacttaggccttatctccaagctcaatgtagcctgagttcaggcaaatatttctgtattcttagaaatacttagcatagcagtgggtccttctcagtatgtggtgccttgcccagcagcaccaacatcacctgggagtttattagaaatgcaaattctctggctcccacccaaacctattgaaccaggaccttcagggatggggcccagcaaacagtgttctaacaagccttccagaggattctgctgaaccctaaagtttgagaaccagtggcatagagattcttctttggccgattttattttctcattaattaaaatgtcacatgcagcacaatctcactgtatcattagttctgagaagaggcttatcaattcatactgaaagggccaacctttattgtatgcttaccaggcagcaggtcttttttcctggttagatcatcacaataagccctggggggtgagtattatcactggtcactgatctgtggagagactgaatgatttccctttggctactggcaggaaagaagcagtcagatccaagaacccatgctcttaatcagactgtaaaagccgcacaacctgaaaatttgtgcattcattcattcctcaaaaagaatgttgcaaaggggagttcccatcgtggcgcagcagaaacgaatctgactaggaaccatgagatccctggcctcactcactgagttaaggatctggcgttgccatgagctgtggtgtaggttacagatctgatgctgctgtggctgtggcataggctggcagctgtagttccaagtggaaccccagcctcctgggagcctccatatcccacagatgcagccctaaaaaaaaaaaaaaaaaaaaaaaagttacaaagggctgtggaaagcacatcaggcttgggagttagacatacctggcgttgaatcccagctagtcatctaatgaaaacaacctcaattgtggagatgagagaaccaggcaaatggtttctgaattccaagtataaatttgtgacctaataagtggaaatattcctttacaaggtctaaaaaataaaactgtctactttcctgaaatagtcttctgcttctggctgacatagaccatgaagtacttgctggggcttggggggagtgactgcaaaattactgtgttataaacatttatgaataaagtagcaaaatataagaaatccactgggaatttttccatactgtttcccactagcaatgatgtatttttatttcattgctgccaggtgtttccattctgtaatccccatgttctgaaaagaactgttgaaccttggtgagcaaaagtgcaatagtttataaaaaagcatttaatttatatcaaaatatggccacacataagaggggagaagatcttcaacagccttctactgttatccttatttctactttgtctaatctgattgtcatcacagtgatcctttaaaaacgtaagagcacatcttttcccccgtttaaacctttcgacagagtttctccccatacttacaataaagcctgcaagtcctccctgagatggtctgagattatctcccaatctcttgctcttacttattcttgctcagtcacagtggatcatttgctgcctcttcaataagtcacgtaagctctggcctcagggcctttgcacttgctcttccctcttccttagtaactgcccagataagatgttcacaaatttttctcaatgacactcttctggttttgactcaaatatctcctcctaagagagatcttctctgaccacctttaaaataatattctccccccgttaccatctatcccaaatccacattttgttttctactcatagtacctatcgcttatctcaagttatacactcacgtgttactgtttaattatccatcactctccacaggaatgtaagctccccacaggccagggttttgtctgatgctctgctgtctccctgacacctagaacagtgctataccagttgttgttaattaacattaactttctgtaatgtttttctttatctgttttcctgtgcagtatgctgcagaataatcacctaagacaggtacccacagaagcgctgcagaatttgcgaagccttcagtcgctgtaagtataattgaaattgcaacgtgttctcaggatccatttgggaaggagcactgaaatatccagaagaccattttagcgaaatgggagagtgtccgttagaacaacaattctttgcatcctcattaaacagtatgtatggggggagactagtgttggattttgggatttcagggcaaagactgaatcagaagaactgcagtgaaaatgctattggagttcccatcctggctcgccggaaatgaatctgactagtaaatatgaggatgcaggttccatccctggcctggctcagtgggttaagcatccagcgttgccgtgagctgtgatgtaggtcgaagacacggctcgaatctggcgttgctgtagctgtagtgtaggtgggcagctgtagctccaatttgacccctaacttgggaacctccatataccttgggtgcagccctaaaaagacacagaaaaaaaagaaaagaaaagcctcttgtatatgttggtatcacaggacagaacacccaggattttgtcctgtaccccctctgtatcatattttacttcctgaattcaaatcaagaacaatactttctcatctagaaaaataagagtcaaaatctggacctctcggcatcttcagtttattctgttttcaagttatctgagttgccctatcatgcaattttgccaagtcgaataaaggcttaattttttagtttattgtatgtacttcaggggtccacaaatcagtaacagagactattggtctctcttaaagtcctactgggcaggtctacaaaccaggagcttccccatcaccacacacagacatgcaaacatcttcacaaacaatcccacagaaatacacatgtgctttagattcacacaccacacacagccgtggtgttggtgttccctactctcaatccacaacttttccgtcgggggttttcaccttcctcagactggaaacctctgaatgaatacaaattagttattcttcacacaaagaactctcttactagctcgatgtttcggagcttgggttgtaagcagctgttcacagtgatgatctggcaagaccatgccatgagtacttaatcagtaaatcttcatctcatcagaatgtgtaagtgtttccaatacattacatgatttctgacatacacatgcatcaacatatgccttcatctcccttactctggaggtcctactctgtcaaccaaaatactaatctctggtctagaatgatgagggaaaggaatgcctttaaaaaggcaaatacaggagttcccatcatggcgcagtggaaaggaatccgactaggagccatgaggtttcgggttctattcctggcctcgctcagtgggttaacgatccagcattgccctgagctgtggtgtcggttgcagatgcggctcagatctggcattgctgtggctctggcgtaggctggcagctacagctccgattggacccctagcctgggaaccgccatatgccgcaggtacggccctaaaaagacaaaaaaaaaaaaaaaaaaaaggcaaatacatattgcaggctctagttgaaaatcactgtaaagaatccatcggtgaataattacacagagtcggcttcatgccttttactgaggtgacacgagaggaggaggaacatgagagctggagatgatactttcttcctttgatctttttttttttttttttttttttaatgaaagcaaatctgggcttgagaccattttaagcttttgactccagtcaaacaaagtgacttcctaggttttcaccagctgagagataaaccagcccacaagagcccctttgtggcaggggatgggtctgctgtgtttttaggagctccttattgacatcgcagtatcactcctctgtgttgcatgaggcttttcctgaaggaaagctacagccagagcagcaaaacactctcgagatgggttgtctgtgtcagggcaaggggtctagtggctggattgaaaagccaacaagataatagcagtggccaataggaactttaaacagctgggaggcaggcagaagatagaaggataagggtttgtaaccaaaagccactatcagcggaaagaagatcacaaacaggacacacatagaagtgaactctttgagaaagagaaaaccaggaatcttttgttcacttgaactgggtggtgcctctctgataaaggtgttcagatttagtttcttacggtctaggtatttactgttcaccaggtgtctcttgcctttggtgtctagttcccaaaaacaattacgtttgaaaccttataagtatgtgttccctgtgaaaaagccaagggttaactttctagtgggatgcttattgcatccaaactgaggcatctagagagatcgtcgtccaaagcacgttggattgtatcggctggttgtgtattatttgtcgcggtccctttgcagagcacagcagaccatgtgaagtggccgtgtgtgctcctacggcgctttgtctcacctttttgtgctaccttgatacaagcgttaagatttgacaaaaaaagcattataaacatactcttcaccaccaactacacagacaccgaactgtaccaacagaaggcgtcccctacccagtttggaggtttgggctgctatgtgccctttccaagtttcactgttgtcccagattccagggatttaatggtacatttccgactttcttcagggagttccaaaaataaaaaaagctttgagtctaatggtcatgacatgcattccttatccgtcttccagccattttccatgcatacctcagccttcgtgccactttcacatcgatacaaataacaaagagggtgacacttgtcttatctatgacacctgtctcatctatgacacctagatggcttccggtcagctacctacccagcttttctcagactactgtcaacccttagacctctgcttagccccttttctaatagttcctttttctctctctcttgaatgcactctattttcctccactttattattttccttgtattctccattcactttttaagaaggactctgcgccccccccccatcaggctggcagtgcactacccctatgcatgcctggctttgtaactcctaaaggggggcttgcaaccctggctgcaaagaaaattgtctggagagttctgcagcccaggctgcaccccagaccaattaaaatcagaatctctgaataggagccccaagcatcagtgctttctaatgggtttaattatatcgttaagtgcttcttcaatgaaaatgggggaaattagtgtcctcctgcatgccttcagatgatctgcaaatatcctcctgaactcttttcaaacctgaaacaataacaaagtccatattgtgcattccactatccaataataatgtatttagaatatgaaataaaaatcaggagttgccttgttttgcttaattgaaatagctcctatctggctcttagttctttgacagtttgggccagataaagttaagaggtaggccatgtgctgcattaccctacgattagaatgtgtccgtggaccagcagcattagcacgtcccaaggcatgcagaagcttgagcctcatctctgtcctacttaatcaaaagcttcattttaacaaatcctcaggcatttgtatgcacagcacttttgagaattaacatttttagtgtctatgacatgaacaaagtctacaaaccaaaaatggttgagaactactgaaacagatcgtcggtattccttgccccacccacacagacttggcttcatggaaggaacttgatatatgctgaaactggtaaccttgctttgaggaagaaacaaattgtactcaagtttcaagggatgcaattttcgttgattccatgtaaacaacaatattttgctttgttcataacgaaagcacacaggtacatttgtgttctttgaacgccctcctgctacttacaaactgctcagctgaagaacaagagggggaaaaaaaaaggagttctggtcgtggcgcagtggttaacgaatctgactaggaaccatgagattgcgagttccatccctggccttgttcagtgggttaatgatccggcgttgccgtgagctgtggtgtaggttgcagacacggctcagatcccacgttgctgtggctctggcataggcctgtggctacggctctgattagacccctagcctgggaatctccatatgctgcagaagtggccctagaaaagacaaaaagacaaaaaaaaaaaaaaaagagagagagagagagaaaacaagagggaagtccacatgccagagcccagtagcccagagcctgcccctgcacagtatgtacattaggtttctgacatggcagtgacggcccagacagcagttggccttttgtaatgtaatcaaatataacttaatttcctttctgttttttggaaaggaataggaaatgtaatcagaattcacagtgaattgtatttcaccaagaaactcttggcattatagtcctttgaggactaggatccaattacagtgatttgatggaaactcaaggcttaaccctcccatcccctcacacctttgtattaagaggtaagaaacagatctatgaaaataatacttgcttcagttgttgattgtcattaatgacattggagcatctcactgtcaattagtgaaagttcaagatgatcaaagagaatgacgtgtgtctgtttgtccagttaaccataatattctttaatatgtgttgcttttcttgccaagtctgcactagcgaaactaattgtggaaaatgtacaagcacaaaaataatgagttggtacatagaaaatgaaaggggaattagacatatctttaatattgtctctcttttccaattctaataatcatctttagagtacattatcttaaaacccgaggagaggcgttcccttcatggctcagtggttcacaaaccctaccagggtccacgaggatgcaggttccatccctggcctcgctcagtgggttaagccaatccggcgttgctgtggctgtggtgtaggccggcagctgtagctccaatttgactcctagcctgggaacctccatatgccacaagtgcagccctaaaaagaaaaggaaaaaaaaaaagagagagagagaggaatggctgcttcagcctggaacatgatctgcccgagcatcttagtgtgatatcagcttaagggctgcagatcccctctgcttggaccctgtaaaatggcaataacttcaccccaactcttcataggagggtttttgctactgatagtccaaaaaaggaagaaggaggagaaggaggtgaggaggaagaggcagggctcacagtgctgaacagaaagtaaagcaagccttgaaccagcagcagttttctccttttaagatccaggaccctattgggtttcagacccaggataaagaatgggaaggaagtaacagaagacaaggagttataagcagacagaaagaaagcagagggaggtaaccaagaggataagattaaaggatcaaaggggttaaaaaaaaaaaaaaaaacaatggctttagtattagtgacattattccctccactaaaatagtgtctggaaagagaaattttaagcctatgtaatacatatacaccacgtttttggtttatttttgtttttcacggaagtgcctgtgtattagctacgtcttgggatggaggtagactgagaagttctagaggccaagagttttgtctgtggcactgtttggagttcatctcacaacccgtgtgcaaaccagcccccagcaaattgttaggagttttgtcttgtccccctgccctcatttaaacatgaggagaaaattattttcaaaggcaaagccctttttccacaagaagagggtactcaaattggagcacagtgctcagaatcagatggaaaaaaaaaaaaaagatgtgactactttaagtcagaccccagacatgaaatattcttatatccgtttataaatcatagacatgacagtcttatcagatagataggtaaggacaagagaacatggatgttatagaatgagaggtaaaatgaactttctttagtcttacagtatgaaatacagattaactttggaaagtaggttgaactctttggagaagctaaaacgatgacagcaaagggaagccacattgcacatggggcgaagtgtttccccccccttgtcaggatagctggggtcttgatgattataaagaggcacagaaagaaccagttgggtataggaataatacaaggacaaaagcagaaacacccctggctataaaagcagaagccatccatatttattaaaacacttacctgcatcatctcacttcaccttcgaagcaacctcatgagatgggagctgagattatcccttcctcagggatgaggaaggaaaaagcccagagagattaaatgaccttctcaaggtcatgtggctggtaatggagtcaggctagaggtgcgtctgaggtaagaagtctggtctttctggctcccacatctgccctttttcccaccacaccgtcttgcctcctgagagaattcattgcagggaacggacactcaacaaaatgcgcttgtgtgagagaaaggagcctgacagaaacacttagcatctctcagcttgtgcttgtggaggtttagaaaaaggagggaaagtagcaatgccacaggacaggcataaataattggagaagagaagagaacgatatttcttcaagtgtgggccagtccgactttcttagtgtttccctatgcatttggttgtctgtttcctcagtacctgggaggtggaacttgtctccactttcctgatgaggaaaccagcagtgagaaaagggtctcttgtccctactacaaaggtaagatgtgactagacaaaggaggaagaactttccacacaaaggaaactacatgtgcacaactttgttgtggggaggttagagggaaactggaacatcagcaaccagaaacaagatcagcgtgacagaagtaggaagagtgaggtggagagaagggcgagaagagcttggtgaggcagatggggtccagaccccttgaaagccctttgggacatgtcatggattttagtctttattttaaaagcaatgagaatccactggaggatttgggagcaggacagggcctgtgttactaggagttcccgtcgtggctcagtggttaatgaacctgactaggatccatgaggacgtgggttcgatccctcgcctccctcagtgggttaaggatcctgtgtggccgtgagctgtggtatagttcgcagatgcggcttgtatgtggcattactgtggctatggtgtaggccggcagttatagttctgatataccgacccctagcctgggaacctccatgggccgcaggtgtggccctaaagagacaaaagaccaccaccaaatatatatatatatatatatattcgtgttactgaagtagtctacgagagaaaggagaactttgtcaggatgggggcagtggagaaggagaaagggaaatgcatttgagaaacatttagatgaaattaccagtatttggtaataagtcgggtgtgaagaatgaaggagaaagaagttagattcctaagttcctggttggtataatcacatgcttactgaagctcatcactgagataagaaatgctgatggaggaccccatttggggaaataatgagtttgtcttgaacatgggtgtttgaagaggtacaaccaactagagttgtcacataggcagcttgcaatccagatctggatctcaaagaaaagtgaaatttaggttaggtctgtctaccatttcaagtgacctaactatacgttgtaatgatttgattgactgccctttagaaaacaggctccataaaggttggcaccatttctacctggttcactgctacatccacagcactaggtacagtatctgcaaagaaagtacccaagaaacacttgttgaggaattcccgttgtggctcatcaggttaagaacccaatgtagtctccatgaggatgtgggtttgattcctggtctggctcagtccaccattgccacaagcagcagcgtaggttgcagatacagctcagatccggcatttgctgtggctgtggcgtaggctggcagctgagactccaacttccatatgccagtgagtgtggctataaagataaataaataaacaaacaaacaaacaaatacttgctgaactgaactgatgcctcagactcttaagtttgatactctaaatagctgcttaggatcagctcatctcccccagtttgaatgtagcctgcatttgctgatctccttgcccccaactcttgcctctgtatcctgatatttcccaccttcccctcctgagctgagtgggatccgtgtttccttcagggatttcaaagctgatctatctacaagatttcgttttcccattgtcttagctgaggactccccacttcaagagtagtgttcagagaagaaccataacattcagtctcaggttgatgagatttggataggcgggagttaggggatattctcaacacaaggctcagaagtactgagaaggagctacagaagagactagactgggacacagaaataagagaaacgtttggcgagataaggtaagctttggatatcagaatggaaaattggggtttgaaaggaagggtaatgtggaagaactgtggattcctaacgggcatgacacagagcccagttttagggagattattttggcagcagcctacaaggtaaattgaagagataagagatcaaaggcaaaagactcagctcagaggctgtctcagtagcacaggtgtgaagagataagaaactagacagtggaaatggacagtctcagagatgaaaataaatggagagacattggtgaatgatgggctggaaagccatagaggatccaagaatgacccccagcgttttttttctaattttatattttattaaactagagttgatttacaatgttgtgccaatgacaccccaggttttaaatcctggtggtagctgggagactaatggtgtccttgacaaaaaggaggtctttgtcttctatggagagaaatgaagctgaggaagtcagtgctcatgttacttcagctatagattatctccatgttaaaaaaaaaaaaaaatcacagcagcggcgacctctgagaaactgagcaacctcatgtaaatcgactttaaaatagacctttcttggagttcccgccgtggcgcagtggttaacgaatccgactaggaaccatgaggttgcgggttcggtccctgcccttgctcagtgggttaacgatccggcgttgccgtgagctgtggtgtaggctgcagacgcggctcggatcccgcgttgctgtggctctggcgtaggccggtggctacagctccgattcaacccctggcctgggaacctccatatgccacgggagcggcccaagaaatagcaacaacaacaacaacaaaaaagacaaaaagacaaaaaaaataaataaataaataaataaaataaaataaaatagacctttctttggagtcttgaacttcccaatgatgccagagagacagaggtttggttctgttagacacttcatccagctactagtcaggtcagctgactagtctgtaaattattgcacttcgataaacctatagactttgggcctccataagctgcagagatgggaaaagcttttttaccttttcagaggcatgtctaaagcaatcatttttcttcctggtttacatcaagtttgccagtaaacaaggtggagaatagaaagatagaagcagaaattctcatatcaggaatcatggtgtttgtatgaattcatttgcttaatatcttccccaggttatagatgggaaatttcattagaaaatggaatcccaccttgttgtcatttcataaatattagaaaacaataacaataaaaatgacattattatgaagagttcctgtaaaccaacactaatgcaatctataaaaaatcatatagaataaaattctatcccagtaatattatgataatgatctgacaaattcaattccagtgacccactggttccaatgcaataaaatttccaagcagtaaaattttatcataacaaagacttcagggacttggtcaaattgtattatatagaagcttggaactacatgcttttcatttcatagcaaaagtaagataataattggaactgctttcgattcttaaaattggataaaagaactaaatcattgtgctcgttggactcttcaggaattgttcagaaggatctcacaaaaggtacaaacgaatgtacaagggagctgagcatttctttaaaacgtacgaaggaaaatgctcatccggatataaaatagcaatgtcaattttatctaagtgatttttttatagatggcattagtgttggtttacaggaactcttcatgtcaaatgctgtgctttatgattacttattgaaaacatccccataagctgtattataccagcagctgcaccacttttcacacctttgcctgttttcccctcgagtcatccacgttaataattgacaatattacagtcatctcagacggggaattagccaagatcaatggcttatcttttgactaacgctttcttccttgtagttaatggctcatcaaggacattctcaaacagatacaagtttgtgtgatacattggcaagggagcttatacaagatatatgcaaagggctatttttctggaaaaccaacagcaagaatggcctcatagattctttaatccttaccataatacaatctataactcaaaaaaatcctgtcaggagtgtggagggtggtgggcaaaaaatgttccttttaaagttgtaaaattaagcttttatttatttgttttggccttttttttttttttcagggccacacgcatgtcatatggaatttcccaggctaggggtcgaaccagagctgtagttgctggcctacaccacaaccatagcaatgcagcatccaagccacatctgagaccttcgctcatggcaacgtggaatccttaacccaccaagtgaggccaggaatcgaatcctcaacttcatggatactagttggactcatttctgctgagccaccacaggaactccaaaaaattaagcttttatgcatgaaaacattaggggaaagacttcagagagaaggtaggaaaataagcttcattttgccattgcctgacactaaaaaattgaattttttttttttttttttttttttttttttgcctttaaccttccagctcctcctcacacgggatgaactgatatttcagaccttggttcacagtggatgcgcaattctgtcttattttgatcttgtatcatactgagtgacgctcatcccggcctggctccgtctgcccagaccaggacattgagtcaccagagcttccttatcttcccttctggtcaccatgctctgtcagaaccacttgtttaacatccttcctacccgccttcctccgtgtttccatccttctttcaagcactcatctcttctcacccagacgatggcagctgccctggtcacttcctctccccctagcatctcctgctcgctgctctcagagtgacctttctacagcacatccattatcagggtgtccagcctggaggcaaaatcagacacacacacacacacacacacacaccccagagaaacagttgcgtgcagatattgaagactgcttgggggagggggagacagttctcaggtcagtaacccagatatttcctggcagtaaagttacccggactccacatgcttaccttggatgctcagagttctcgaagcatgtctgggtgaggtggatggtcctgcacagcatccactagaaacagccccccgcccagcggtttgtgccttaggatcccttctggccattgtggagccctctgacctccacacctccctccaaagaagaagttctttcatcacaggacccaaagtcatgacaaatccatttcttttttttttttggtctttttagggccacacctgtggcctatggaggttcccaaactaggggttaaattggagctgcaactgccggcctacaccacagccacagcaaggagggatccaagcctcgtccgtgacctacaccagatccttaacccactgagcgaggccagggatcaaatccgcatcttcacaggtgctagtcgggttcattaccgctgagccacaacaggaacaacgcgacagatccctttctcctcagctgtggttttctaagttgcattgcagaggttcatagttaatataaattctgtccttgcaccagatgatgtcatattttttcctcagctgttttagcctttggggccttttaggacctttgctggaggtaacacacaagaggtatccgactccttttcactgtggtgaaaggcttcctgtcacactaatcaaaacatgcagtgctgaacgtatgtacgaaggaggatggtgaacagacactcagccgtcctgccagtccaccatatgaccaaaaacaaacacgtaaacaaaaggcaatctcctaccaagagatctgaaattgcaaagggttaagatccttcagaaaacataaaagaaagtttctcagggggatgatactatttcctaaatgtgaagatttttctcaattacgaaatcctatccttcatagaatgctgtactgtttccaaaacccttccacatacattttctcatttaaatctcataacaattttgttgcctgttgtaattctgctttaccgatatgaaaattgaccctcagagaaattaactgctatatcccctgatctcagaataagtgacagcgtctgggtttaaatttgtcataattctcctacttcggtgcactttccagtttaccataggctcatcgtgacatagcaagtagggtaaaagtttattttatctatcattttttgtaagttataagtaacatgcagttatttatatgggtgccataaaataggtgccagttcccaggtatagatcattttcagattttgtttcaagatcacaaagaagtttgtccgaaagaaggtgggaccactcccattgttaaagtgctttattttgctccacttgtctgcatgcaggttttttgtcaccttattgatggattcttaaatgccaaggtcattgctaggaactttctgagaatgcattgcgttgcatgtcacctcttctcctttgccttcagagtgaagtttaatattcagtaatgtatcaggaataaaaactatttgccctttgcgataagatatatatgtgtgtatataatagattatatcaatacacatgtatacacatacgtgtacatgtaatgataatactggaagcagaaaagtgagggtgacttctatcaagtctagctgcagatgcctcttcgttatttcttttacgggggatgaaagaaggagtctgcatttctcagatctgaaattctctcacaagctgaccctcatttcaaccagcttttgactaagagaaaatttgagtttactcctttttttcggactggtttctatggtgcaaagacaaaaatgcaaactgttaacccacttcaagtaagagtaatttaaaatcgttaaggttctttatagtttacaaagcttcgttgtattactttctttgattataaagcaaccctataagatagagaggttgtctccctgcccctgccacttacagctgtgaaagctgaggtctgttgacttgaccatggtcatatagctgctgtctgacttagacccaaaatgtttcatcccaaatctctgcactaattcacagattctaagctgcctacacacacacacacacacacacacacacacacacacacctttgtgacttgtaggaaatctaaataacaaagagctacataatataattgtttttcttgcttctgatgatgttatagaaccaatttttatgtgtattttcatgtactgtgtactgatgtttttcttacactcaagtgcttaaatattgcatttttcaagcatatgaatagaaaatgcccagctacccccaaagaccagagtgacttaatatgagctcttcatttactcactgaataggcacaacttgtaccctgccagctgctttgtccccagttttaaccttgttctgtaagaaccacctgtatgagtggctggataattaattccatactctggtttgccttgggatcttctgtgaagtttaaatcagataactcctctgagagtgtaccactctctaagataggtttggaccactgcagccaagtgaacatgagcttgaaatcactgcattgagaaggagtttcatttatggggagcttctttgggggggcacacccgtgacatagggaggttcccaggctagggatctaatcaagagctgtagccgctggcctacaccacagccacagccacaccagatccaagccgcgtctgcaacctacaccacagctcacagcaatgctggatccttaacccactgagccagaccagggatcgaacccatgtcctcatggatgctagtggggtttgttaaccgctgagccacaacaggaactccctatggggagcttcttatatgaagaagctgttgaaaaagtgctcagaatacctgagtactgggccttcgtgttttagtatttgcagtgttagaactgagaagaattgccctgttttactcatttcatttcagattacaagatagctctaagattatgtcgtagaaacaagattacctccttaatactgggggaggggagtgattcataaaagatgtctatccagagaaatggacctttcaacattggaaactgacgccagggtcacttggagatccagcagaaactttggtagtaatttaaatatcaaagccgacaagcgtgtgactccttgcaaagagccttttctgtacgtgtaccctgacgcttgcactgaactcgaaggtcagtcattttcagcgcttagaagccacttgtcatttcaatgcataagccacaggcaataatcagacaacacatcaactaaaactcgaaatctcacatcctttaaataaagtcttcatgctataatcattgcaatcgttaaggagagtttacatatgcgctttttttttttttccccttgtctttttgccatttcttgggccactcccgtggcatatggagttcccaggctaggggtcgaattggagctgtagccgccggtccacaccagagccacagcaacgcgggatccgagccgcgtctgcaacctacaccacagctcacggcaatgctggatccttaacccactgagcaagggcagggaccgaacccgcaacctcatggttcctagtcgcattcgttaaccaccgcaccacgacgggaactcccatatgtgctttcctagtcaaacagaaaccagcacctcctcaccccctggtgaaattataccttatttgaacatgggttgagcatgtggtggctggagcaagggggagagacggtattgttttcctaaatgtttccagttccatttggagacagaaccttgaggcatttggaagctgcatttctgctgcctaacaatgatagctgtcctggaagtttaactgtgagtgggctgtgaaaccagacttgtttgttccaagtgcccttatctcggtagcaacacttggcccagggccttgagggcctccctccccattcagtcaggagaagcaggagccagagcagtggggaacacgtgtgaccaaaagtgaggacgccccttccatcgagattagctccctaccacagacagcgctctctctttctaaattgtttaaatttggtttgcaggggattattccgtgatcctggtgtgcttcctgcatctgtttagtcttaaatgcttcctacagcatttttcaaaatccactcgtgattttctaatccttcctaatacaggaaagttgtttaaaatgcaactcttcttacttttttttaatcctggctttgaaaatgcaaactaatgtgtgaaccttaagaacagattggaaaaaataagtgagggttgttggatgcagagttgaatgtcggtggttctgtcgtttgtacacctttcagaaaatttcaggttgtgtctgtttggtcacttcaaaatgacaagttaaggcattgtgattagaggcactgtttatgttggatcggagtgaaataaatgtgttgcactccctctgctggctctgaagctgcaacaacttattaactagacctattttcagatgtatttccccgtttaaaaaaaaaaatggtgtgcaggaaaagcaagcacaaactagaaagctgctaattttattattcgtttaaaatgtcaagaaaaactaacatctgctcaatgccattggctaggataagcaaaaaagccagaagattttaggcttttcagcactgtactaagaaaaagggagaggccacctccccctctccctgaggcataactccaacagccttgtaaaaattactcggtccctttcagatcctgaaataatcttttctcagaccttaatgaacaaagtagtagatgagttggctttgtatgttgaaagcagtccaggaaagcaacagcatctagttggcttggggtgggagggaggggagaggatgcctttctccccgtgtcaaaaaggaaagctaaaagatcatcactcggaaaccttataccccaggggaggccacttccttgcaggaaagcgtgttacccatggtcctggaggagaatcacagcagtgtggaattttacagacaagggggcctaagcagcaatgcttctactcaggagtgagaagagaggctaccaggagaacagaatgccccagatgtcggggaccggggcgggggcggtcctgagggtcccaggccctgggtttttgtttgtttgtttgtttctattttgggtggggttttttttttttttttttttttttgctttttagggccacagcaatagcaaatggaagtttcctggtcgagttagaactgcagctgcctgcctgtgccacagctgtagcaaggccagatcctttaacccactgagtgaggccagggattgaacccacatcctcattgacgttatgtcagattcttaacccagacagccacaacaggaactctttctttctttctttctttctttctttctttctttttctctctctttctttctctttccttctttctttcttttttctttctttctttctctctctctctttctttctttctttcttccttccttcctttcttccttcctttctttctttctttctttcttctttctttcttttttctttctttctttcttccttccttccttccttcctttctttctttcttattatttgtttatttacctggccctgtttcaatcttggtaattagcatattgagatcttcttgcaaggtccgtcttctggctgggctttgctctcttttccactcttgctcactgtcttccacaggcgcctggatgccaaccacatcagctacgtcccccccagctgtttcagtggcctgcattccctgaggcacctgtggctggatgacaacgctttgacagaaatccctgtccaggctttcagaagtttatcagcgctgcaggccatgaccttggccctgaacaaaatacaccacataccagactatgcctttggaaacctgtccagcttggtagttctgtaagttttattgattttttttttcctctatcatttaacagtttctaaattcactctaaggccttgaccttaaactgatagcccttaaactttgaatagggacattttaaggaaagaaaaactccccaaatgactcataaatgttctaattgtataaaagaattaaacatttatggagcacctgttaagtaccaaccactgctaggcattcaaaaataataaaatacgacctctgtcttaggggagcccattgtatattaaacaggtaatcaccacactgtacagttagaactataactggttttaaaaaatcccttattcctgataataatttataattcatgtcaagttgaaatatgtcccttttgttatttagtaactacttcttgaaccccttccaaaggaacagaagcaagaagaaacccttgacaatgagccatgcatttgtctagcttctttcctttgaggtctagctttcattaattttgagtaaatcgaaatgattctcaagtaagagtttgaaaaataatcagtttaaaagtgtagagatatggatttcccctgtggctcagtgggttaaggatctgacattgtcactgcagtggctgaggtcgctgctgtggtgcaggttcaattcctggcctgggaacttctgcatgtcgcaggtgtggccccccaaaaaaagaaaataataagcaagaagtgtagcagtatggcgcatgtgtacaatataatagttaatcattgaaacaacttgaaagtctctgagatcagcttgcatgggaagttccagtaaggcatcttcttgcactatttcaagcccactccctagcccactgctgccttccacttttctcaatgacacatctgtctcttgcactgcagtaatgtactcctttgagctctggccagcactttagccatctctttaggttcccctcaatccctttccccatgtgcttcatataaaagatcctcaataaatgtcatgtagatctcccatgaaaccagcatgtatttattagaatgaaaaccttagcctgaaatggtagagcatttggggtgaccttaacaaatttctatagtgaatttcatttgcaaaggatgatgcttctaacccactagaaactgggtaggtgctttaatttttccctccactagaggaaatggatttacaaagatttcttcttttctgactatcctctttaagccccctttccaaaagtccctgtgggacctggtccagttttcagttatgctcacagctgttctcactggccctgatctaagtgacttaccattgagccaaagatgataattagatacattagtgctgtggcaagatagctaatctccttgggcagccgaaaaatatccccattctctgcggctgaaattgttgtttcctgttaacctgagtccccaccctgagaatgtagccaggagaaaccatcagctttaacagtaaatgtgcttagtgcaccttggccttttcctccatcctccatttgtctgtgtttgtcagaatctctgtgtaacatcctccatcccaaagccatgctgaacaacctgaacttactttcatcagactcgctcactccatgaaatctcaaacccaaaaggtaaaactgtgactgcctattaaattctgtggcataggatcgtctgtggtaccagtgaatgggcgtatagcttttaaaacaaaatgaaacaaacaaaaaaagttaaactcagcatttcttatatatcatcagtgcattctttagcataagtttctttggatttcagcaatcaaaacgccatgccttcgtttccccatgcggtgccgtatgccttcatgactgtctgtaatattctcctgcaggcatctccataacaatagaatccactccctgggaaagaaatgctttgatgggctccacagcctagagactttgtgagttgaccttttattttgtttccctgttttcagtgttttcatgactatcctgaaggaatcgaaatagccttcaagtcaaacttgctatttggtttggttaattgcctgagctttaaacagatatttacagtggctttatcttacccctccaacacacaccctacagagctgaacaataaaagcgagattttattttaatctaacaagagtgctaaattagaatcattataagctaaatgagacgtcctccactgagcagtgactgtttgcttaggaatgtggactctgtgtcaaggattagttctgtattgtcccttgggctcactattcatgagcctggtcctctgctctgaatgagaggatgcagaggaggcaaatgccagggtctccaattggagaggaacaaacattgtattagccaacagtggagattcctgctgaagcagctcaacagtctaaaaagtaataacagctgccatttactgagtgtatctctaaccagatattgcactgagcactcaaccacatgtttcagtccatcctacaactaacccctcctatgagagcaggtatgactatcccattttaggctctagtaaggttaagttaatttaccaacccagcatagcgcatcagcagcataactaggattggcttccttctctagcttgactccaaaattcaagttcaccgctatattctacttccgaagaggggctcaaaacctcagttctcagttgatacctagactgaaaatacgtagaggccagccaggtgactgcccagagcctaatcccaggacagttcctatgggcctctctggggctgagctgtttctcctgagaggggctgctcttcattgccctgtccagtgagaggggtatgatgtgggtatggtgacaggcagtgaggatgctgagagtgccgggagaagagggtggttccagctgtgctctcctctcctcctgctttaaaaaaaaaaaaaaaaaaaaaaaaaatatatatatatatatatatatatatatatattgctatttggtttggttaattgcctgagctttaaacagatatatatatatttagggggatggggtggctgtggtatgcagcagcttaatatgggatctcagttcccagaccaggaattgaacccgggctgcagtgaggaaagtgccaaatcctaaccactagaccactggagaactcccttcacctgcatgttttgtttttgtttttgtttttgttttttgtctttttgcctttttgtcttttcttgggccgctcccgcagcatatggaggttcccaggctaggggtctaatcagagctgtagccaccggcctacgccagagccacagcaatgcgggatccgagccgagtctgggacctacactacagctcacggcaatgccagatccttaacccaccgagcaaggccagggatcaaacccgcaacctcatggttcctagtcgcattcgttaaccactgcgccacgacgggaactccagcctgcatgtttttaatactcaaatgtgttaagccacagtgggctgctgaatctacaacactggctctaactgggactgcagaggttgatgaccaaagaccacttctcctatgggcttggggcccattgttcgtttgcacacagagtttagcaatttgtacatggtcaatctagcttctaaccagatctgggtccattaatttcctgagggaccggacagttctccttccagtgttacttaagtcactccatgttccctcttaaaaggaaagaatcggagttcctgttgtggctcaccagaaactaacccacctaggatccgtgaggatgtgggttcgatccctggccttgctcagtgggttaaggattccggtgttgctgtggcctgtggtgtaggtcacagatgtggctaggatcctgcattgctgtggctgtggtgtaggccagcagctgtagctccgatttgacccctagcctgggaatatccatatgccgcagacgtggccctaaaaagccaaaaaaaaaaaaaaaaaaaaaaaaaatgaaggaaggaatcaccttcataaagagatgctgaaatgaacttgtccaggcagcactttgaagtcaacagaatggcctaggcaagccattcctatcaactgcttggaaaaattagaatttctcctttatttacacgagggctggctgggaatctcagcaatggaaagcaccttccagtcctttcaacacaagaacaacaaaagatgagttctgataggtatttcccataaatagtctttcttcctttccccctttcccatttgaggtaaggactcaagattacaaattcaggtaattcatgtattcagagcaaattcaaatgagtctcgctgtcccctctttcctcgccttgcccagggctatagaggatcagtaaattggtaactcccaaccccttcctttccctcactagaaatccaagtaatttataaggacctaatcctcatttaatcaaaacagagcatctggagagatttacaaacaggaagaaaatttatgtccacaaaagacacgcgtttcctgcctgctgtatagcttaaggatggtttgtgccagacaagtaggaatcttgattcatgtttaaccccgtcaaattatctctctcttaaaattaaatagcagttcttccccacagaggaagatctaaccgcatctgattgcctgaccaaaaggtttttctgcgtcatctgggatatctgccattcccagagaccaggaggcaattactttttcatatgaagatgtagctataaggagaagaaagtctatacagcacttgtgagtaatggaattgaaaagccatttgggccttaacagagaaatgaatgatttggggtctgttggccattccacgtctgtgtgtgaaggatgacagagagctctgcctcataaaccaagggcaatgtcatccttcagatctgaatccttatctgttaacgtgtggaaattctagacctggaatccttcctgaaagtaccctggaccacataaaacctctgcagaagcaggttggcatcctggacctcaggctgggaaagacacaggaactgtacaagagtcagggacaaagccgttcaagatgtcctctccagagagttcttcttttggcacagccgaaatgaacccgaccagtatccatgaggatgcgggttcgatccccggcctcagtcagtgggttaaaggatctggcattgctgtggccgtggagtaggccagcagctgcaactccagtttgaccactagcctgggaatttccatatgcagcaggtgcagccctaaaaaaagaaaaaaagaagaaaaaaaaggtgcactctgaaaagcagtgagacaaagtcagagggcagaggagggaataggaaaggggatgtggttcaggaacccgaggagggcacagactagctggcagcttggtgagccacatcagcaagccactaacacccagccaggggtctctcccacggagacattcacactgccctttttctgtgtccctgccctttttcttcaatgcccagtgcagttgagacttgactcttttaaatttctcatgatcagtctgatgaccattttaatgtagatacttgggggatgacggtggtcagcaagctaacaaagtctctctccgtaaagagaaggaatgagaattgttgtcaatgatactaatccattggaagagtatgttaactggtatatatgaatttttatttttttatgtttttatatatgaatctttaaataaaaaactgaacatactatactattttcttattttcagagatttaaattataataatcttgatgaattccccactgcaatcaggacactctccaaccttaaagaactgtaagtagaacagacttagtgggcagactctattctttgtgaatttattttaaaatgtttgactattgtatataatacaacttactgagaatttttttttttttttttttttttttttgctttttagggctgaatttgtggcatatggaagttcccaggctagtggtcaaatcggagttgcagctaccagcctatgtcacagccacagcaacgttagatcaagccgagacgaacctacaccacagctcaaggcaatgccagatccttaacccactctgatcaaggccaaggatcaaatctgcatcctcatggacactagtcaggtccattaccactgagccacaataggaactcttactgaggaaaattttaaaggtcatataaataatggttaactacatccaaaatatattttaatttgattatatcaccaaagaacagcaatcttttttttatttataataggtttctctaatatatgtacattaggaaaaaagatacaaggagtctcctggtggcttagtgggttaaggatctggcatattgtcactgctgtggcttgggttactgctgtggtgtaggatcgaagcctggcccaggaacttccacgtgccactggagtggccaaaaagaaagaaagatatatatttctctaagccatactcttttcctaagccatattgccccaacatttgtatactttatagatctgtagagaactatattggattcagttatctgttgagcatttcaaaagacaaacttttctaggttgcctaaggctaggactggatgaaagcaatgtatgtgcccagtatagtgcttgatgctttcgcatacattgcttttttctttctttctttctttgtttttgtttttttgtttttttggtttttgttttgttttgctttttagagccaccctcaggcatatggaagttcccaggctaggagtcaaatcagagccactgctgctggcctatgccacaaccacagcaatgcaggatctgagctaggtctgtgacctacaccacatctgctcatggcaatgccagatccccgacccactaagcgaggccagggatcgaacccacatcctcatggatactagttggatttgtttccgatgagccatgacgggaactcctcacatacattatttcatatgctttcacgcatgcttcataaatcctatttaattgttaaagaaacccacaattttactatgataaggctaactttcagctatgaggttgcatacattgtctttcatatattttaatttaattagcctttctttgtacctaaaaatatgaataacctatgcaatgttctaacacttggaaaagaaaataaacatagagcttattctcttggcagagagaaataagacacacagccaaaggaggtagactgcagggcttgttcataagaaaaagcctctatggggttctaccttaggaaaccccacctggggcttagctgggaagtcctcctgatctgggtcaggcagcccaaaaactgccactcacctcattcagccacagacccactgaggacccaaagacccttgactctgcccctctgccagtcagacaacatttgcaccgacagatgagaatcaatttattacagactcactgagctggggcacccttcttcagctcaggaaattttgtcctcataaggagattacttaaatctctacttcactgcagcttaagtcaattttctctcctcctagcttcaaatggggtcaggaggagcgatcataatcctctctccaaagatggtttcctctgtctcaaattgttctagggcggtgctctattggatcttagcagatggattttctgtttataacattagactgtaagtttctataaaacacagctgttattgtcagattcttttttcctctttcgagatggcagcttgtgatttctttttctcctcgagactaagtgagggcatgtaagtttcaaaatgcataggtgagttcagccttatcctccagttactgaaagtgctgattgttgccacagtttggggcagactattaaatgatattaccctacaggttatttcctataaattattttttcctctctttctagaggatttcatagcaacaatatcaagtccattcctgagaaagcctttgtaggcaacccttctcttattaccatgtaagtggccatagttctttccagtttttctatcctgaaacatagtgtgcattgcactttcaaatataatacgagtgttctatatttgtgcaagttttgtctcaatcaatgtaaggatctatgccgagaagtgtacatggaaatgtttggccaggttttagctgaatgctgaagcaacagtaaagattcttttgcttaggaatgtgcattgccttatgtttgctatctttaaatgccttgttccacttcacgataatcttatgaaatgctattttgcttatctctattctctaaggaacaaaactgatttttagagcatcaatgtcaaagtcacctaatcattcagaacttaaacccaggtatactagaaacataaatcattatcttaaatgttctcacccccccaaaagataaaaaatgtgatagttaattaacgagatggggggaatccattcacaatgtatatgtatatcaaatcaccgcaatgtaagctttaaatatacaattttatgtcaattatacatcagtagagctgaaatataaatacatacatatgttatatacaaataaaggtcctttattgcaaggagggggagatgaggagactactgttttatgctaagtctctaaattcatgcattttccagcccaaacattgagtcacatttataccaacaaggatgtaattctagaaaggtgcatatgttctagaaaagtgtttatgaggatttataagccacaatgtcataagataaatttactacttcaaagcatgattagctctatgtttcacatttagcacaaacggaaagttgatcatgtgcaggtatacaggaagatggaaatatgtaacgattaggttggctcttaattaaaagacacctggcaggtttggtgtatagtattggcctgaggtcttgggtacggggtctggaataaggaggcgtaaggacaacttttgagcatgactctaggggcacttcttggcagatatccgtcctgtgccaacttgattgagaggctttctggaactgtgcagcatttgccacaaatgagatggaatcccaattagccctgacaactgagaaaaaaaataccattcatcatttctgaaatgttctgacagtatgttggcaaacgaagggtgttaaagtttattgccaataaacattagttctcattagagagtatttacatactgaaaagttggcagatcgcattgcctctctttgcccatttttcccttgaagccacagaacaggttttcttaatcactcttttgatatgttaatccccgttgaagaggactaatgcccccaatatttattagcacttctattcaccatttgatggccttgactctttgttttctcccggaaacgcatccctccttgcttctgcctctgtttgactcgatttctttgcttcccatgtcctgttggctacccgggcctcatttctacctcgtagtccccgtcctagccataactccttttcatcccctctgcctccagcccctctgctagccagacctctgattttttaccaatctaaggctcatctcctggcctctaccactagcaaagctgtcaccctgaaagcacagcatccctattgtcattctcttgctgtggaacctgcagggtcggcttgcctgtttacattaaacgccagctccttgccttggcattcccagcttcccacatggcccaagccaaccaaccctcctgaccggttccccaaagcttagctcagttaccctatgcataacccagaccagctaagctatattcatagaagaaagctctttttttgtttcctttgctcctatgattccatttccttgctatgcttgttgaaattctgatttccagggtccatttctaatgacatttttcgtggaatttttctctacctcctgagagaactccctcctgagaacctatatagctccttctttgtattggtgatacttctgccttatggacaagcatgtgtgtccatagctacccaatgtacttacctacatacacgtatgtgaacacaattatatacaaacaagcatagaggggataagagatgatgtgatatttggaggaaaactctttttttttttttttttttggtctttttgtcttttctaaggccactcccgcgacatagggaggttcccaggctaggtcgaatcggagctgtagctgccggcctacaccagagccagatccgagccacatctgagacctacaccacagctcacagcaacgccagatccttaacccactgagcaaggccagggatggaacctgcaacctcatggttcctagtcggattcgttaaccactgagccacgacgggaactcaggaaaactccatttttaaatcataaggaagttcccactgtggcgcaatgggataaaccatgtccggggaatgatgggacacaggtttgatccctggcccggcacagtgggttaagaatccggcattgccccagctgcggcttaggtcacaactatggcttagatctaatccctggcctgagaactccatatgcctcggggcggccaaaaaaaattttttaaatataaaagaaattgtaagacctagagtccagggctagccccttctcttaccaattatcccagccaagggaaaagaggtcagaggagagaatacagaaggggctttaggaatgtcaagtgacagctgtatcccaggcactgtgccacatgttccatatgaattagcttttttcatgtgcacacaaaccctagaactaaatattattacagcagttcgataaattggaaagttgaaagtctaggaggtaaagaacatacccaaggatctaagtctcagcttcttcctacaaaaatgaggaaaacattatctaagtgtctacaccatgggtcttcaaagttgtcaggtggatcagttaagctctatatgtgaaaatgctttgtgaaccaaaaagtcagattccccttatttatttgttttatcccaactgattccaagccacttgggcgaggacaccttatcattcttcttcacatccccagcagctagcatgatgccaggacataggaaacacccaatgacagcttccttaacaaatcagtctttaaatgcaaagcagtacctcctctcatacttctagcaggagagacaccaatagatattgtcaaatttaattgaactggaaggatgtctcagctaagaactatttgtgggactccactagtcaggcagttcaaataccatgacatggacaacatctgtaacacaaaatatgttttggtttctaataatgtcttaccttttagagcaatggttaattttttttttttttttttttttttttgccttttctagggccactctcgcggcatatggaggctcccaggctaggggtctaatcggagatgtagtcaccagcctacgccagagcctcagcaactcgggatccgagcctcgtctgcaacctacactacagctcacggcaacgccggatccttaacccactgagcaaggccagggatcgaacccccaacctcattgttcctagtcggattcgttaaccactgcgccacgatgggaactccagcaataattaatttttaaaaatctaatattacgaaaagccagagtagcaaatttcctctcaaaaaatttaagactcctttgcatatattaaaagatttgaccggaggttatgacccttatgtaaatatatagaatttcatctttctttagctagtggtatacctaccagtatcttgaattaccttttgccactcaacagaggttccacagatgttagaagcggatgaagagttttaagtatcacttgcttttctccttagtagctaacattcatatctgccttgctgtcagacattttcagttttggagttcctgctatggtgcaatgggatcggtggcatctctggagcgccaggacacaggttcaatccccagcccggcaaagtgggttaaaggatccggcattgccacagctgctgcataggtcacaactgtagctcagagctgatccctggcccatgaattccatatgcccaagggtggtcaaaaaagaaaagaaagaaattttcaatttcacatttagtgtggatttcttacccaaagtcccatgatgtgagtaaaattacctttgagatttgcctgaaaccaagtaatcaggaatgttatgtagtatagaataatccaattatataagtacatcttatatattttttgaaattttgaacagttattcatttttcactcaagtgttatactaagacgcgtttggatttttttcagacatttctatgacaaccccatccagctggttgggagatctgcttttcaacatttacctgaactaagaacactgtaagtgtttatttctctcacagatcacttgcccctttcaaggctgctgcaaagaccagatgaacattttcagttgctttgcaatggccataaaagtgaccccttcctaggatcttctccctgtaatattcatgtatgcctgcttctaggactttgaatggtgcctcacaaataactgaatttcctgatttgactggaacagcgagcctggagagtctgtaagtatttgagtaagctcttgaattggcccagagcatgcacgacccttcaagcctgaccagtcttaacatgagtgaatcaaaatatgtcaccgtgtgatgcctgaatacaagaatgatgtctggtttgtgtttcaacaggactttaactggcgcacagatctcatctcttccccaaaccgtctgtgatcagttacctaatctccaagtgctgtgcgtatcagtaaggccataatgttgtgtaaacaggggtgaaatgacagacactatttcaataatcctttaaaaggggagaaagttctttccttgcaggtcttcaggaggtcaggcctcctttgccccctttgggacaaggaagtggctctcagatatgaccttggtaataatggcagtgcaccccttgtttctaagtcccaaacaatctcccagtagagtttcaattggcacacatttcctttcagaaaatggatgtgagcaagccaaaataacttcatttcccccctcccgaagctctgaagagcaaaaaagaaaaaggaaaaatatatatatatataaaatgggtcaaaacatcaatatagatgtcaaaataaaaattattagggagtttctgtcatggctcagcagtaacgacccaactaggatccatgaggatgtgagttcgatccctggcttcgatcagcagattaaggatcccttgttgccatgagctgtggttgcacgtcatggacgcggctcagagcccatgtggttgtggtgtaggccggcagctgtagctccaattcaacccctagcctgggaacttccatatgccacaagttcggccctaaaaagcaaaaaaaaaaaaaaaaaaaaaaaaaaaaatcaggcccacacaacccttgatagcaagcaaacattcttccaggatagctgtaggtgccatgttctaaaatttcttctgatggctaaacagcttgcatatgttctttctgttgatgtggggcttttccttctgcccttcgaagagccttgtgttgaccttccatgttcagccctctcctgccacaccctgttatcctcttgagcatggctgggacctaacaaaaggagccttagtctctcgccatggctctgttctcacatagtagacttctttggcagtggaatgaatgcctgtgattccaccacaagcctgacccagtagcagaactacagacagcacagtggaagaatgggaatatcccttcagcgattctgccttccccaaccccagcccactatgtccctttcagatgtcggttgctcagtgacaagagttgcaatcacaccacgatagccttggagtcagtcactcaggtgtaaaggaaggggaactagagcaagcttcctcttcttcaccccacgttcaccctaggaggtgtagatacatgagaaaaatgaaaactagcggagtaaagggaacatctgtcaaaatcaccctgcaagaaagtagtggagttcaaattcaaacacaggcagtttgattgaaaagttgggagaaagcacctgtacaccaggaggtggaaaactcaggagccccaccttaggattctgcccaccacacccaaaatagatataattacccttatttcttgaggaaactggttcagtaacatgaccagaattttacatgctgtaactaataagtagggagccaggatttaaatcgcacctgtctccactctgtcacacggtgaacgggaaaccacgcgggtctagagctgcatacttggaagctgactggcaaaccaattaagagcaaaggctctagggtctatttttctggttcaaatccggcctccctgcatactctcacagaatcttgagcaagttatttattccttccagggctccgtttcccaatctataaaatagagacgtagccacttccttcaggggatcgtcctggggattaaataagttcctacatgtcaaacacttacaccggcacctggagccttgtaaactctcagacaatgttggcctttatcatcgtctttctcacttcctctacaccatgattctttcaagtggaaaccttgaagggcctgaaagaacacaaccacattaacggccgtgtcttgccctgttcaaagccacgtccacctgtggtattgatttgttcttcacaagaaccctgcaagatcagcaaagcaagtatcttcttgtttaaggaggggactgaccaagacatcagtgcccggttcagcgtatcgaccaacttgaatcagttaaagtcgagcagaaccctgggtcctgacctttgctccactctgctttatcttctggagtagaggtccgagaccacccttcctttggctgaagcatccagagtagctgttccttgaggcaatgtcacggtacacgcgtgcatttgtctccaagtggcttctcctccgtgagaatgacctggctgtgtgtggcttcccccacttaccagctggcgatcttaggcaagtcattttggtttgcacggggatcatcacagtgcctccctcaaatggtgaccacaaggattaaggaaatcaggtcaaggtgtaagcccactatctatctataagacatgcttgacccgtgttagccatgaaaatcactaggtggaacatagataagaggtgacactgtgtctttagtagaagaaggccccatgtatcctgtcctaaaaggtaggcaggaaggcttggtccccttgcagtctgtggagatgagtgaaccaaattatgatcagctttgataacatgtatatctattgtatcacagtcactacttttcagaaatacaaatcatactgcttatcacatgtatttatgcataggtgtggggtgtggttttggttttttgcttttgctgtggtctttcaaacctctggtatgaactacagatttgtgtttgcacatagcttcacctgagttacttctgtgtagtcaataaactgtatttctatgtaagttccatttcttcaataaatgtaagataaaagatcaattccgagaataaaataatttaaaatcctctcagaaaacaatatgggtttttcacttgcatagtggtgatctggttttaagaattagaatcaggcatgaaaatgagttagttgattagctgataacttttaaaacatgagacaagacttttatatatctatgttactttatctaccagctttcttttttaaagctttctgcaaattaccatggcttgttttagattagttatattatactataaatatgtccagatgtttttgttattctgtaatttgttgagttttggctgaagacttttttagattttttctttcccccttgtaatgattgatttcaaagctagtgctatattttgaattgttgttgggaatttttttttccgttgattagaaaagaaccaacgatggttctcttagctgtggagatatagcagctcttagaaacagaggcatagtaatttaatttaatttgtactcgtttctgtaagataatagctagaacattgtagatcatcactaacaaaatataaattcattaagtaaattatttaaatgaattaaagaagtataacagttacattttggtaaacatctgaatctactgcttaatccaataatgcctatgactacctgctccgtgtcagggctggggaaacattcagagtctgcctttgggagacagatcatcgtagaggggaaagcgaagcaaagcatgtttccttaagattaatatgtaacagattttcccttctgtaccttttgtttttcagagatttatcttacaacttgctagaagatttacccagtttttcagtctgccaaaagcttcagaaaatgtatgtctgtaagagtctctaagtcacctagcaaaaaccaagagccaaaactcacatttccaggggtcattggtgtgcttaccctgtggttcactgggaagacatctgatgctttttcttcttacgcacacaagctctgactgtataggatggctgtaaatttgggagacatggatgatattattttaccatgccttatactataagtaaagcctttattatggtttttttttcctggtatacctaatatagtaatgaactgagtttgtttgagctttggtatttttctaaacttatacctccaatgagtagcattattttttaatatgacgggacttgtgtttgcttcccaatagtcaccttcagagaaggtgccctgtttgtcgtatttcaggtgccaccaatgctccttaaacatgaaagattttagaattcccctcttaaaattgcctcctctatgaaacatcagccttgttgctttatagtcatgttttggttcttgatcaaaaaaatattaaccatctatcctttcctacttaatttttctctgaatgcttttggacaatcaagggaggggaaaaaacccatctttggaaggtaaagatttgacgcttctgaaaacgctcaaagaatataagaaaggctcaaaggcagagccaggagatgcaacctcaaagtgtttgaatgatgttggcattttagaacatagggagttccctctgtggctcagtggttaacgagcctgattaggatccatgaggatgtggattcgatccctggcctcacgcagtgggttaaaggatccagcattgccgtgagctgtggtgtaggtcgcagatgcagctcagagcccacgttttgctgtgactgtggtgtagtcctgggaacgtccatatgctacaggtgcagccctaaaaatcaaaaaaaaaaaaaaaaaatttaagccttagattcacccaaaggagctagctgctttgaaagagagacaatacttttttaagtgtataaatcgttctttcttaaataaaaagacatttttttccaatggtcacttgagagtcatacttcatgtggtcctgtgacagagggcaggtaattcctctgtgaaagttctctgaagactaatctttgaaaccagtgactttgaaatcaatctttggaactctgtcatttcagagacctacgacataatgaaatctgtgaaattcaagcggacactttccagcagttgctcggcctccgatctctgtgagtaccgcttcccagtaccttcccaacatagagcactgcttttctttttcagagcttcccctaacattttcttttcaccgtaatatgtgtactcttgggcctgtgtacatgtatttgctatgaaaaagcaaacattttgctcatgatcatttccaggaacaagttcaaaagtagtaaatttgcaatctgctttctgccagtcagcagacatgaaacttcgataaagaggaaggaggccaagccatcgaatagcctatgaaatgtgtttctgttcctagcctggacttggcagagactgttttgcgtacaagactgggatgaccacacagtccttcccgtctgttttgctttgctttcctggtcaatttttcaatgtgagtctctctaagttcttaagagtcacctggaggtgacggtaaaaaaaaaaaaaaaaaaaaaatgctgagtccaggttctcacctgcctcatgagttggattctctggagtatggcctgagaacctgcattctaacaagaaaggtttgagcacaactgtcctggaaaatgcctctgctgcacagactactaagatttgtaaacattcaattccatcgtatgattgaaaatactacatattaaccataagtttaaaaaaaaacctcggtaacagacttgtggtgacagttttaaaaaacttttccatcctggaactgaactgcagtgtaattttggtgagtgacgacaatgtataatgtttaaactgctgtatgtagtttcaaaaaacctttaccttcctaaagtttttcttaacatttaagagattatactctcataaaataatactgttcctcaaaacattttttttggccacccccgcagcatacggaagttcctgggccaggaatccaacctgcaccacagcagtgacccaagccactgcaatgacaacactggattcttaacccactgaaccccacgggaactccacaaaacactttaaattctcctttaagaatcaccgtctcccaaaaatttgcattgaaacctaccttgctctgaattaaattatgtggccttttccttacttttggtcttaaatcagcttgtgaagttcttttattttttaaaatacatgtgcaacaaaaatacattacccacttttataatctgctttactcacaggttggagttctggctgcttccaaaagttagttctttttcaaaaggttgatatatttgataacactgtattgtataactgtatttgcaatgagagtagaacttaaatatttttcaccaaagagccatggaatcactcacctgaatcaccatcatcctcctcctaactaacctttccaccatcagtccctacccatttatctcatgctccaaagagaatcttaagtagaaactgaatgatgttacttcccttcttcaaaactcttcatcgagatgcagcggcaaaatgaatccgactaggaaccatgaggttgagggttcgatccctggccttgctcagtgggttaaggatccagcattgctgtgagctgtggtgtaggtcgcagatgctgctcggatcccgcgttgctgtggctgtggcgtagcctggcagctatagcaccaattagacccctagcctgggaacctccatatgcctcaggagcagccctagaggggaaaaaaaaaatttccaaacactttctgtgcagatcagggattctgaagttcgtcagcttgagtttgaatctcagctcctgagattactctgtgacctgaagcaagtcacttaatactcactcggtgctttatttcttaccagtaaaatggggcgaatgataatatctacgtcctaggtgtgttaagggttatgagataatcctgtggggggctggatatatgcaaaagtattattattgttattgttattgttatcactattagaacaagtatatatccttttgggtatacaagtgagggttctttcaaaggaatcagaactcaccccagatggttcaaatgaagggccatctgtgtaggtactggaagcaagggacaggatgcagaaaccagcaatgtgaccaagactggggctgaagagacacagagaggaatgaatggtgtttccaaaacccaggaagagctgaggcatggaagggggatgccttggttgagaaagagacaagattgcaaaacagaggaggagaccacaagtgcccaacctctctctcctccaggggcccctcactgggaaccaagaactgtggggagctctgcatgcagtctgaagggtccgggcaagagtggaactgattcccccagcactgttcacaaataggaatggaattacagtcctctgagcatataaattctcccatattcacttaaaacgcagtcaaatcgcagtagagccccaaggcaaaacctggcctatgttcctgatgttctgctctggaagctcaccaaggcacccgtgtgccactcagagccaccagcaagactgacagatggcagggcttgcgtccctcactggacacgtgttggatgagcagctccatagaagaaagcctcctctcgattacccacttgtagcttgcccactccttccaggccattccttcccctccttagctggaactttcttttgttctgagatcaatgaaaggagcaaaataaaaagacttccggtttaatcttattttattatttatttatttgtttagtggacacactcatgacctatagaagttcccaagctagggggtcgaatcagagctgcggcttctggcctatgtcacagccacagcaacagcagatccaagccacgtctgtgacctacaccacaactcacagcaatgctggatcctcaacacactgagagaggccagggatctaactcacatcctcgtaaacactatgttgggttcttaacccacggagccacaatgggaactccaaagctttccggttttccccagtttagggggcctttcaaatgagaatgtaaatattttgtgaataccatttatcctgggtaacttagggggcccaatactgtgaataaattacctattcgtgctagagaaagctgccagggatcactttcagttcctcagatgtctacattttagttccccttatgagttatgcattgtaaaaatacgaattttaagtttattgctctttttttaaatacggaagttcttattcctgtgaagttatcattatatatatgtctggggcaggggggagtgaattttatttctaataaatgctagcctctctttccttcatttctcagttaagaatttaaatgtatgtcccctctaccgaaaatgtttttgaaagtatttctgtgttgctcttaaagtctgacatacatggacaatgtacacaaattcttgctgagttccattgcttgtccggggacattttataacataaaatttacttgcctgtcagaggaagagcctcagaagttcccgtctcttccgactccccaacccccactcatcttaaaaagaccctaataaaattttacaaccatgttaaagtcaactttttagcaagaagagagtaaaaactaaaatatccacctgaatgatttccctgaattgatttaaacaaggttgctttaaaattctctctttacccattttttgctgtgaatatctgcatctgaaggatgatgtggtaagctgtttctgtcttgaaggtaaattaattttttttagataaatgaatatatacggtaaaacttcacgtggttttcttttctataagcctctcacttgccctttttttcttttttaataggaatttagcttggaacaaaattgccactattcaccccaatgcattttccaccttgccatctctaagaaagctgtgagtatttcatttgtttatggtatatcttttaataactgggtaccatctatcacagcctagtaaaaatattggttctgctgaagaagcccacagggtacagaatgcccagtcctcagcttcactagagctggatcaaaccagaaaggagcccccctgcccctctagggtcatgggggcccttacgtggataaatgaagacctgggtgacagctggttctggttactgaggtgaagtttttcattttttttgctgagctatcaggtggaatggaataaaaatgtcagttcagtgctaaagggctttcattttacattttgccctcttgactaacagaaaatctccctgcccatcttctttttcctccctctccacatctcgctccttcctctcccccttcttctgcatctcacagaggaaggaggaattggtcagacagaagttaatgcgagaaggaagaaaataaacccctgacaatgttgtccccctccccctctaacttgatcccgtccgcatcacttttcaaattagatgcagcacctcccccttgttttgagatgtccccacctggcaaaattcagctgaatagagaaattacaatttggacgtgattaacacatttttcctcctgcaattctggttgtaaattaaagaaaagaaaagctgtcgggagctaaaaccaaaatggttcttcgtaccgtcaagcgtaaggaccctgcttagaagtttttatggagcagtttaataatctttaggttgcttttttaacataatcgttttatactcacaccgactaaaagccaattgttgtttgatgttttgcagggacctatcgtccaaccgcctgtcgtcctttcctgtaactgggttacatggtttaactcacttaaaattaacaggaaatcatgccttacagagcttgatatcatctgaaaactttccagaactcaagtgagtttgtcattaaaactaataagatacatttgtggtcatgtgaaataatagatgtgttagagtggttttcaaaatttatgggcctcttttgaattgcatttcattgccttcaagtcccccacccctggggcattgataggacaagccctggggtgggagatgtgtcttcctgaccacatttcctttctttctcaaagatgccacctggaatcacagagttaatggaaagaatctgaaaaccaatgatccatgggtataaactagcatatgtaattaaataacattataacataattgtactcctgtttacattttaaaattcatctatctgtatatgtaggttctaataaaatggactaaattaatattggtttgaggaagtaatgctttttaaaacctgttatgcttattagcaataaagaagtcctttgaaagattttacatatatatatgtatatatattctatatatgtattttaagccacaccactttttaacctgcttttaaaaccataactccaggagttcctgttgtggcgcagtggttaacaaatccgactaggaaccatgaggttgtgggttcgatccctggccttgctcagtggttaaaggatctggcgttgctgtgagctgtggtgtaggttgcagatgcggctcagatcccgagttgctatggctccggtgtagggcggcagctacagctctgattggacccctagcctgggaacctccatatgccgcaggagcggccctaaaaatggcaataaataaataaataaataaataaaaccataactctaaaagagagttcagaaaatagaagagagaaaacaaagtctaaaaataccccctaaacaactctttatgttatttccttccagattttatttccagtgaatttttaattgttagaatcacagtatgtatacaaacctgtatcatgtttttgtcacttaacattatgtcataccataaccactttttaatggtgctacaatttacctagcagtcacttcttaatgattttattatggaaatttccaaacacaaaagtagggaaaacagtataatcaacattttatggcaataatttgaataactacattgttattacgaatgaattgcactgtaatcatctctatgcacataattaatattttagattgtttattaaggctaaaattcacaggcagaattattgaatgaaacactatatttaattatctatttctttaaatgtatgtttttgtcttggttttgttcatttaaagggttatagaaatgccttatgcataccagtgctgtgtatttggagtgtgtgaaaatgtctataaaatttctaatcagtggaataaagctgacaacaacagtgcagatgaccttcataaaaaagatgccggaatgtttcaagttcaaggtaaaacctactatgtcactgtgatgaaactgtgtctgaaaggggcttgaaaagtcatcaaacagatccactgaatgccagacaaggccctatgtaaccaatcccactttttaaagattctcagagaaagaggaaacccctttaggattcattctaaaatttcatcttttccgtaagtgaattcttaataatacctataattagatcccatttagcatacattgtcctcaggggtaaatattattattaatattttcatcaactttaaagagggccccatgccatgtggatatctttttccgaaatgctcctgagcatttttcttcaggagagaaaatccattcttattcttccagttggaggaggtaaaagatacagacatttcagatgcctccatatttcagcagaagggcttaagaatccaagttcttatgcaagcaatcctaacacgtccttccctcctaccccccacagggctgtcaggagagtgaaatgagataatgtctgtaaagcacttggctttccttgaagtaagacctgacttaaatagacaacaatgcacctcaaatcatacctgtggtaatgaggaggtgccaggcagctcccactctcctttccctgccaaccctgcaagtttgcacagagtctaaacagaccggctcttcttcctttgggtgttaatgatttgttgattcccaagcaagtatgattcagtctgtctgcacattatcttgacttttcttcctgcgcacctttctgattaaggagctgatattatggcccttgatttttttttttgttttgttttaggttacagcatgtgtcttgctcttgttctggctttcatttttgcttttcctgctttacccaccctgatttctactatgtttattcacatacacactctcctcaccaacaaaaacaaaaacaaacaaaaacaattagagtgtctactatgtgccaggcactgttctaggccccagagacacacaatgaacaaaagggacaaagttcttcaaggagtttattttcaagggcagtgattctcaacccttgaccgaccctagattggaatcacatggaagctttttaaaaacactgatgtttgggagtccccgtcatggctcagtggttaacgaatcagaccagcatccatgaggactcaggttgaatccctggccttgttcagtgggttaagggtctggcagtgccatgagctgtggtgtcagttgcagacgtggctcagatcttgcattgctgtggctgtggtgtaggccggcagctacagctccaatttgagccctagcttgggaacctccatatgctgcaggtgcagccctaaataataataataatgatgatgatgataataaaataaaaacactgatattcaagccatacaccaaaacaaccaaatcagcctctttaagaaagtgcatgagatcccaaaactcctatttcatggatagctaatccaagcaccaagcagatttgtcctctgttaagaactccccagaaaagactgaaacttacttattcgccctcttgtgttcagagaacacagcccagaacatctgcactgtagccatctatgtattagaaaattggataaagtggttcttcactgaatggcgaggtccacactctctcgctaatttaggtcagtgcaattaggagccagcgtgagctccagggaacaaaaacagaatgagagagcttcaatgttatgaccaggagtccagggttattgtcctttgcacccagttctgctggaatactgtgtcataaagtagcttgagtagaacatccaaaagctacttaataatagacttgcaagaaaacgcagggagaaatgggacgtctgaccttcagttccctttgtgacatttcatgtctctgggacaatctaggacactcctccctgggagctggtccccgaaaagagagagagaagtaatcattccagaacttcttattcctgcagatgagcgtgatcttgaagatttcttgcttgactttgaggaagacctgaaagccctacattccgtgcagtgtacaccttccccaggtgagaaaagcatcagacctgcctcagcagcagtaaccccccttcattctaagaccatcaatgacattttggctacacttcaaagcaaaatctgtgcacctttgcagctcatcagtatctcctggggacttttaaaaatacaaatgtttgttccctggagttggcctccagtctagaatttttgtggaagacttttgatttggatcaatgggaattaaagaaacagtaatgtagtttagaagtaaatatgatttttaaatgatattagcaatttagcctgaacaggtctgccactgtcttgcttcatctaatcactgatttttgacagtgatttctttgaggtcttccctaaatagctatttggttaaagcaagtaatttttaagggtaagtacaaaatatccatatatattgttcacttaattgtttgtcccattccctaaaataggagaggccagttgaaaaaaaaaaaaaaaaaagacagctgggttgatattttcttttccacaatttgattgttccttgctcccaaacatcaaatgtcagtctaaacctaaaaacattttcataaaatgcaccttcatacaactcatactttctcttctctaatctgagcagcagctgtgacctatgccacagctgctgcaatgccggatccttaacccattgtgccaggcattgaacccagcgccacgacacagacaatgcaggatccttaaccctccctgtgcagtgggaactccttacagaacctttttgaagcatcctggaaaaatataactaaaaaaagctcaggagacctggctttttccctggccccaacactgctgtatttgagggacctgaatcaagtgatttaaacttcctgtctccatttctctgggaatggcttgaaaacctgggcatgtggcatgttcacctttacattacatagtggtttaataaagtgaagcatgccccaagggaactgtgtatgaccatggctgttactggcctctatctctcagtgtcctaataagagaaacaaaacggggactgggcaaaagttgagaaccctaactgatacccactctttttttccttggacttctaggccccttcaaactttgcgaatacctgttaggtagctggctgatccgaattggagtgtggaccatagcagttttggcacttacttgtaacaccttggtgacctccacagtgttcagagcccctctgtacatttcctccataaaactgttaatcgggttgatagcggcggccaacacgctcatgggggtttccagtgcggtgctggctgccgtggatgcctttacttttggcagctttgcgcggcacggagcgtggtgggagcagggggtcggttgccaaatcgttgggtttttgtccatttttgcctcggaatcctccgttttcctgcttactctggcagccctggagcgagggttctctgtgaaatgctctgcgaaattcgaaatgaaaactccctcctccagcctgaaagcagcccttgtcctctgcgccctgctggccctgaccatcgcggcggttcccctgctgggcggtagcgagtacagcgcctccccgctctgcctgccgctgccctttggggagcccagcgccatgggctacgtggtggccctcgtcttgctcaattccctttgcttcctggtgatgaccatcgcctacaccaagctctactgcagtttggaaaagggagacctggagaacatcttggactgttctatggtgaagcacattgctctgttgctcttcaccaactgtatcctttactgcccagtggctttcttgtccttctcctctttactgaacctcacattcatcagccctgaagtaattaagtttatccttctggtgattgtcccgcttcctgcatgtctcaatccccttctctacatcctcttcaatcctcactttaaggaggatctggggagcctgggaaagcaaacccccttctggacaagatccaaacacacaagcctgatgtctattaactcggatgatgttgagaaacagtcctgtgactccacccaagccttggtacccttcaccggtgccagcatagcctacgacctgccttccaattccgggtcaccggcagcttatccaatgaccgaaagctgtcatctctcttccgtggcatttgtcccatgtctctaattaacatgtgagagaataaggtttccaaaattgaaaacccaaaaatgtgagattgagtacatcagagtagtaactaaaaagagatgaatggaaacttgattttaaaaaaaatcaatctctcagtgtcaaaaagctgagaattattgatactggagagtggaaaacaagtctaaatgctgcttatataatttgttccgctacgatagatcagtcacacgattcaggtaagcctaaatatatataatatttaaaaaacagattgcaatgttcaaaaaattctccatgatttcaatggaattcttttccagctcactaatttaaggatttgctgggggtgggggggtcacttacttacaaaaaagattttaaacttgaaaacgctatgtttggaggtattaggaaaataaggattttaaatggcatgtgttactaagtaatggttgtgaccagtaggatttcattttaacgaccatatggagaggttttagtactcttccaccttttctcagaaaggatcctccaggtaaccagtcttcctgtgaatggataacataaggcactattcattcatattcacatcatttttagtgatgctagattttatgtatcggcactagatagttccacctgtagtagaattaaactgcttacaaatatttggaaaggaaaatgaacttaaattcttcaattgccgtgaaggcaattgtgtacagatgaggtttgccttgtctaattcatgtttaacttcttggtgcccagaggtcagaagaaaagtccattgccagcaatggacatacttagaaaagatggctagcaacctgggatgtcttttccccctgggtgaatgcacatttttcgcagaaggttttatcagttgattcagactgatgtgcatcatgaggtaattatcaaaggcagacacaacataaatgaaattcactgacataacttcgcacatgtatctctagtagctttagcaacagtgtctgaaaccacttttgactcaacagaaacttggcaacaccttatcctgtctatttagcttggttttagctatgttgtctttggatcagcccacttgatgtcaggaagatgacttctctgcttattccatatttaatatacatgttaggtattataagaggtaaaattattaaatactaaaggccgaagggtaaattcctaacttctattattctacactagcttcaatacatccaaaccaaaagactcttaggtagatttatttttatataagcatgtttattttgatcagatgttttaatttggaattgagaaaatacatttatgaaatgttttataagatgtgtaaatatagaactgtatttattactacagtaaagattcagtaacactaaggaccatgataatttgtacagtggcatattcttccatatatatgttgtgtttctctgtccattttctttaaattcattaagtgtatgtaatatatgtaaatgtatagtatatgtaaatagataccaaacttgcttttctattgggtacaaaataaatttgtaataaaaatgtgtgactttaaagcaaaatatttcaggtgttttattgaacccataggcaaatatatgccacctaaatgtttttgatttt

(19) United States
(12) Patent Application Publication
(10) Pub. No.: US 2022/0095597 A1
(43) Pub. Date: Mar. 31, 2022

(54) TRANSGENIC MODELS FOR STEM CELL THERAPIES

(71) Applicant: North Carolina State University, Raleigh, NC (US)

(72) Inventor: Jorge A. Piedrahita, Raleigh, NC (US)

(73) Assignee: North Carolina State University, Raleigh, NC (US)

(21) Appl. No.: 17/425,626

(22) PCT Filed: Jan. 24, 2020
(86) PCT No.: PCT/US2020/015008

§ 371 Date: Jul. 23, 2021

Related U.S. Application Data
(60) Provisional application No. 62/796,437, filed on Jan. 24, 2019.

(51) Int. Cl. A01K 67/027; C12N 15/85; C12N 5/071; G01N 33/50

(52) U.S. Cl. CPC A01K 67/0278; C12N 15/8509; C12N 5/0628; C12N 5/0678; G01N 33/5073; A01K 2267/0393; G01N 2800/20; A01K 2227/105; C12N 2510/04; C12N 2015/8572; A01K 2267/0331; A01K 2267/01; G01N 2800/7028; A01K 2227/108

(57) ABSTRACT

The present disclosure provides compositions and methods related to the generation and use of transgenic animal models having stem cell reporter systems. In particular, the present disclosure provides a novel transgenic animal model that expresses a nuclear-localized fluorescent reporter in cells endogenously expressing a leucine-rich repeat-containing G-protein coupled receptor (LGR) gene (e.g., LGR5gene). Given the role of LGR genes in stem cell and cancer biology, the transgenic animal models provided herein are useful for a wide range of therapeutic and diagnostic purposes.

### CROSS REFERENCE TO RELATED APPLICATION

This application claims priority to and the benefit of U.S. Provisional Patent Application No. 62/796,437 filed Jan. 24, 2019, which is incorporated herein by reference in its entirety for all purposes.

### GOVERNMENT FUNDING

This invention was made with government support under grant number OD019738 awarded by the National Institutes of Health. The government has certain rights in the invention.

### FIELD

The present disclosure provides compositions and methods related to the generation and use of transgenic animal models having stein cell reporter systems. In particular, the present disclosure provides a novel transgenic animal model that expresses a nuclear-localized fluorescent reporter in cells endogenously expressing the leucine-rich repeat-containing G-protein coupled receptor 5 (LGR5) gene. Given the role of LGR5 in stem cell and cancer biology, the transgenic animal models provided herein are useful for a wide range of therapeutic and diagnostic purposes.

### BACKGROUND

Leucine-rich repeat-containing G-protein coupled receptors (LGR) are expressed in a wide range of stem cells including, for example, skin, gut, lung, ovary, mammary gland, kidney, pancreas and liver. In particular, the LGR5 gene has been implicated in cancer and LGR5 expressing cells have been referred as the cancer stem cell. Being able to track and study LGR5 expressing cells will not only facilitate investigations into their normal development, but also help elucidate the function of LGRs during cell regeneration and repair, as well as their roles in cancer initiation and progression.

### SUMMARY

Embodiments of the present disclosure include a non-human transgenic animal comprising a genome that expresses a nuclear-localized reporter gene in at least one cell type or tissue type that also expresses an endogenous leucine-rich repeat-containing G-protein coupled receptor (LGR) gene.

In some embodiments, the endogenous LRG gene is leucine-rich repeat-containing G-protein coupled receptor 4 (LGR4), LRG gene is leucine-rich repeat-containing G-protein coupled receptor 5 (LGR5), or LRG gene is leucine-rich repeat-containing G-protein coupled receptor 6 (LGR6).

In some embodiments, the nuclear-localized reporter gene is a fluorescent reporter. In some embodiments, the fluorescent reporter comprises at least one of GFP, eGFP, mCherry, CFP, BFP, YFP, eYFP, photoactivatable GFP, dsRed (Discosoma species fluorescent protein), inFruits (mutants of dsRed), TagRFPs (Evrogen), eqFP611 (isolated from sea anemone Entacmaea quadricolor), Dronpa (photoswitchable fluorescent protein and EosFP (photoconvertable fluorescent protein).

In some embodiments, the nuclear-localized reporter gene comprises a gene encoding H2B. In some embodiments, the nuclear-localized reporter gene comprises a gene encoding an FLAB-GFP fusion protein. In some embodiments, the nuclear-localized reporter gene comprises a gene encoding an LRG protein, or fragment thereof, fused to a nuclear-localized fluorescent reporter. In some embodiments, the nuclear-localized reporter gene comprises a gene encoding an LGR5-H2B-GFP fusion protein.

In some embodiments, the gene encoding the LGR5-H2B-GFP fusion protein comprises a gene encoding H2B-GFP downstream of the LGR5 ATG start site. In some embodiments, the gene encoding the LGR5-H2B-GFP fusion protein comprises a gene encoding H2B-GFP downstream of the LGR5 ATG start site, and upstream of intron 1 of LGR5. In some embodiments, the gene encoding the LGR5-H2B-GFP fusion protein does not contain an IRES site. In some embodiments, the gene encoding the LGR5-H2B-GFP fusion protein comprises one or more fragments of SEQ ID NO: 2.

In some embodiments, the at least one cell type or tissue type that expresses the endogenous LGR gene is a stem cell. In some embodiments, the at least one cell type or tissue type that expresses the endogenous LGR gene comprises skin, eye, inner ear, gastrointestinal track, uterus, ovary, prostrate, mammary gland, kidney, liver, pancreas, cervix, and/or placenta cell types or tissue types.

Embodiments of the present disclosure also include a cell or cell line derived from the transgenic animal described above. In some embodiments, the cell line is a primary cell line or an immortalized cell line. In some embodiments, the cell is a stem cell. In some embodiments, the stem cell is capable of forming an organoid when cultured in a matrix

Embodiments of the present disclosure also include a method of screening an intervention for a disease or condition. In accordance with these embodiments, the method includes: a) contacting the transgenic animal of claim 1 or the cell or cell line of claim 15 with a candidate intervention; and b) determining the effect of said intervention on a disease or condition in the transgenic animal.

In some embodiments, the intervention is selected from the group consisting of a drug, a lifestyle change, an alternative medicine therapy, or a combination thereof. In some embodiments, the disease or condition is cancer. In some embodiments, the disease or condition is associated with stem cell function. In some embodiments, the disease or condition is an intestinal, hepatic, renal, lung, or skin disease or injury.

### BRIEF DESCRIPTION OF THE DRAWINGS

FIG. 1: Representative schematic diagram of the LGRS gene locus of a transgenic model of the present disclosure.

FIGS. 2A-2B: Representative diaizram of the LGRS genomic targeting strategy using CRISPR (FIG. 2A), and representative gels identifying two colonies as positive for the H2B-GFP knock-in (FIG. 2B).

FIGS. 3A-3B: Representative images of transgene expression in the duodenum (FIG. 3A) and the jejunum (FIG. 3B) of the transgenic lines of the present disclosure.

FIGS. 4A-4B: Representative images of transgene expression in the skin (FIG. 4A) and the biliary tree (FIG. 4B) of the transgenic lines of the present disclosure.

FIGS. 5A-5C: Representative images of the co-expression of LGR5-H2B-GFP transgene protein (FIG. 5A) and mRNA (FIG. 5B), merged in FIG. 5C.

FIGS. 6A-6C: Representative images of LGR5-H2B-GFP transgene expression in locations consistent with endogenous LGR5 expression in various stages of the hair growth cycle, including anagen (FIG. 6A), catagen (FIG. 6B), and telogen (FIG. 6C) stages.

FIGS. 7A-7C: Representative images of LGR5-H2B-GFP transgene expression in individual epidermal cells (FIGS. 7A-7B), which correlates with endogenous LGR5 mRNA expression (FIG. 7C).

FIGS. 8A-8D: Representative scatter plots from FACS experiments of GFP-positive stem cells expressing the LGR5-H2B-GFP transgene in liver (FIG. 8A), biliary tree (FIG. 8B), pancreas (FIG. 8C), and small intestine (FIG. 8D).

FIGS. 9A-9B: Representative bar graph results of organoid formation efficiency (FIG. 9A) and size (FIG. 9B) from LGR5-GFP-positive hair follicle stem cells.

FIGS. 10A-10B: Representative images demonstrate that LGR5-GFP-positive cells plated in Matrigel containing growth factors formed skin organoids (FIG. 10A) and biliary tree organoids (FIG. 10B).

FIG. 11: Representative images of LGR5-GFP (“hi”) organoids stained with various epidermal markers.

FIGS. 12A-12D: Representative images of low resolution cytoplasmic LGR5-GFP expression in a mouse model (FIGS. 12A-12B) as compared to high resolution nuclear LGR5-H2B-GFP expression in a porcine model (FIGS. 12C-12D); GFP expression is overlaid with DAPI staining in FIGS. 12B and 12D.

FIG. 13: Representative images of LGR5-GFP expression surrounding a wound, demonstrating increased cell proliferation.

FIGS. 14A-14C: Representative images of LGR5-GFP expression in hair germ cells, the primary proliferating hair germ cells in anagen (GFP expression is overlaid with Ki67 staining in FIG. 14B, and DAM staining in FIG. 14C).

### DETAILED DESCRIPTION

Embodiments of the present disclosure provide compositions and methods related to the generation and use of transgenic animal models having stem cell reporter systems. In particular, the present disclosure provides a novel transgenic animal model that expresses a nuclear-localized fluorescent reporter in cells endogenously expressing the leucine-rich repeat-containing G-protein coupled receptor 5 (LGRS) gene. Given the role of LGR5 in stem cell and cancer biology, the transgenic animal models provided herein are useful fbr a wide range of therapeutic and diagnostic purposes.

The use of a large animal model having anatomy and physiology more complementary to humans provides the ability to recapitulate human disease and overcomes many current limitations of rodent models. Thus, the transgenic porcine models provided herein are a powerful tool for the study of translational aspects of adult stem cells of the gut, skin, liver, and kidney, among other cells and tissues. These swine models can also be used to address questions regarding the role of stem cells in epithelial/mesenchymal replacement during homeostasis and intestinal, hepatic, renal, lung, and skin diseases/injuries, including cancer, and regenerative responses resulting from bums, wounds, radiation/chemotherapeutic or viral/bacterial damage.

The transgenic animal models described herein include a chromatin bound/nuclear-localized GFP fluorescent tag in the LGR5 locus that can facilitate the study of adult stein cells, adult stem cell progenitors, and the stem-cell niche. LGR5 is a critical marker for multiple adult stem cells, including but not limited to, stein cells present in the skin, GI tract, liver and pancreas, as well as those present in lung regeneration, and mammary gland development and repair. Essentially almost all epithelial barriers involve an LGR5 progenitor cell as a repair stein cell. LGR5 has also been implicated in cancer, and it is upregulated in many caners. LGR5-positive cells are often referred to as cancer stein cells, as they can be the initiator of many types of cancer. Thus, LGR5 is important from both a regenerative medicine standpoint, as well as oncology.

Previous investigations supported the use of IRES elements and cytoplasmic GFP in constructs used to aenerate LGR5 transaenic mice. Embodiments of the present disclosure used similar approaches, but these were only partially successful. Additionally, cytoplasmic GFP is very difficult to evaluate, both quantitatively and qualitatively, and it can be challenging to differentiate cytoplasmic GFP against a fluorescent background signal. Conversely, nuclear localization signals, such as H2B-GFP, are much easier to score and provide an enhanced degree of accuracy and spatial resolution. Also, the H2B nuclear localization tag (chromatin-specific) is particularly useful, as it allows for lineage tracing studies and investigations into chromosome dynamics by identifying the timing of when LGR5 aene expression is activated or deactivated.

More than ten transgenic mouse lines described in previous investigations using LGR transgenic models; however, none use H2B-GFP as a reporter and all include the use of an IRES. These existing mouse models have well characterized mosaic expression patterns, and cell ablation studies are limited due to the suboptimal penetrance of transgene expression in these models (see, e.g., Stem Cell Reports; 3:234-241 (2014)).

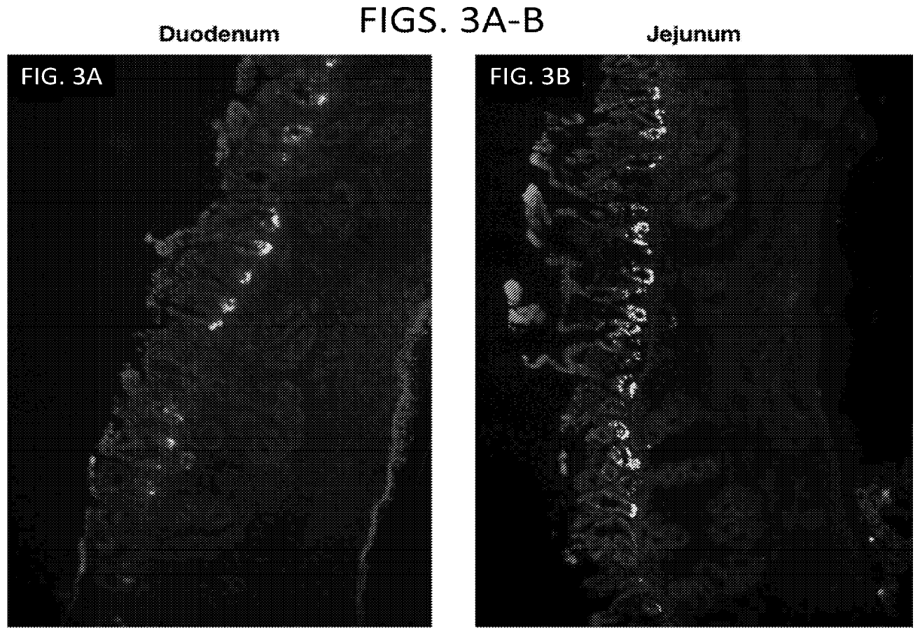

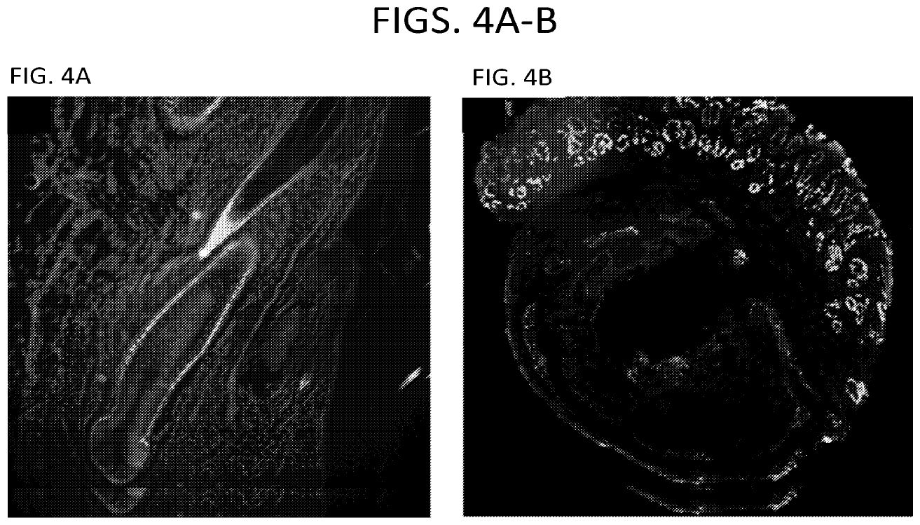

Thus, in contrast to previous studies, embodiments of the present disclosure include at least two elements not previously identified as important for replicating LGR5 expression in a transgenic animal modelremoving the IRES and switching to the chromatin bound GFP. Additionally, embodiments of the present disclosure include transgenic construct in which the transgene has been inserted into the 5′ end of the transgene (e.g., adjacent to the ATG start site). These features make the transgenic models of the present disclosure much easier to score and have led to the identification of unreported LGR5 cell populations in certain regions of GI tissues (see, e.g., FIGS. 3 and 4), including cells that are not epithelial in nature, which may be more consistent with LGR expression in humans than other transgenic models.

As described further herein, embodiments of the present disclosure include a non-human transgenic animal comprising a genome that expresses a nuclear-localized reporter gene in at least one cell type or tissue type that also expresses an endogenous leucine-rich repeat-containing G-protein coupled receptor (LGR) gene. In some embodiments, the endogenous LRG gene is LGR4, LGR5, or LRG6. LRG genes/proteins are a unique class of evolutionary conserved seven-transmembrane receptors characterized by a large extracellular region that includes multiple imperfect copies of a leucine-rich repeat protein interaction domain. The extracellular domain mediates ligand binding as a prerequisite to modulation of downstream intracellular signaling pathways via heterotrimeric G-proteins.

In some embodiments, the nuclear-localized reporter gene is a fluorescent reporter. In some embodiments, the fluorescent reporter comprises at least one of GFP, eGFP, mCherry, CFP, BFP, YFP, eYFP, photoactivatable GFP, dsRed (Discosoma species fluorescent protein), mFruits (mutants of dsRed), TagRFPs (Evrogen), eqFP611 (isolated from sea anemone Entacmaea quadricolor), Dronpa (photoswitchable fluorescent protein), and EosFP (photoconvertable fluorescent protein). Other reporters can also be used, as would be recognized by one of ordinary skill in the art based on the present disclosure.

In some embodiments, the nuclear-localized reporter gene comprises a gene encoding the histone H2B gene, a chromatin-specific nuclear-localization signal. In some embodiments, the nuclear-localized reporter gene comprises a gene encoding a nuclear-localization signal, such as but not limited to, a monopartite or bipartite nuclear localization signal of any other protein that is localized to the nucleous. Examples include the following: SV40 large T antigen, nucleoplasmin, Antennapedia Homeodomain Protein Antigens, Nuclear Ki-67 Antigen, Ku Autoantigen, snRNP Core Proteins, Ataxins, Ataxin-1, Ataxin-2, Ataxin-3, Ataxin-7, BRCA1 Protein, BRCA2 Protein, CCAAT-Enhancer-Binding Proteins, CCAAT-Binding Factor, CCAAT-Enhancer-Binding Protein-alpha, CCAAT-Enhancer-Binding Protein-beta, CCAAT, Enhancer-Binding Protein-delta, Transcription Factor CHOP, Y-Box-Binding Protein 1, CDX2 Transcription Factor, Chromosomal Proteins including histones, CCCTC-Binding Factor, Centromere Protein A, Centromere Protein B, High Mobility Group Proteins +, Methyl-CpG-Binding Protein 2, Minichromosome Maintenance Proteins +, Polycomb-Group Proteins +, SMARCB1 Protein, Telomere-Binding Proteins +, Tumor Suppressor p53-Binding Protein 1, Fanconi Anemia Complementation Group A Protein, Fanconi Anemia Complementation Group D2 Protein, Fanconi Anemia Complementation Group E Protein, Fanconi Anemia Complementation Group F Protein, Fanconi Anemia Complementation Group N Protein, Hepatocyte Nuclear Factors, Hepatocyte Nuclear Factor 1 +, Hepatocyte Nuclear Factor 3-alpha, Hepatocyte Nuclear Factor 3-beta, Hepatocyte Nuclear Factor 3-gamma, Hepatocyte Nuclear Factor 4, Hepatocyte Nuclear Factor 6, Host Cell Factor C1, Immunoglobulin J Recombination Signal Sequence-Binding Protein, Inhibitor of Growth Protein 1, Katyopherins, alpha Karyopherins, beta Katyopherins, Mad2 Proteins, Mediator Complex, Cyclin C, Cyclin-Dependent Kinase 8, Mediator Complex Subunit 1, NF-kappa B NF-kappa , B p50 Subunit, NF-kappa B p52 Subunit, Transcription Factor RelA, Transcription Factor Re1B, Nuclear Matrix-Associated Proteins, Heterogeneous-Nuclear Ribonucleoprotein U, Lamins +, Nuclear Receptor Coactivators, Mediator Complex Subunit 1, Nuclear Receptor Coactivator 1, Nuclear Receptor Coactivator 2, Nuclear Receptor Coactivator 3, Peroxisome Proliferator-Activated Receptor Gamma Coactivator 1-alpha, Nuclear Receptor Interacting Protein 1, Oncogene Protein p55(v-myc), Poly-ADP-Ribose Binding Proteins, Apoptosis Inducing Factor, Ataxia Telangiectasia Mutated Proteins, CCCTC-Binding Factor, Centromere Protein A, Centromere Protein B, Cyclin-Dependent Kinase Inhibitor p21, DNA-Activated Protein Kinase, DNA (Cytosine-5-)-Methyltransferase 1, DNA Topoisomerases, Type Heat Shock Transcription Factors, Histones, Ku Autoantigen, MRE11 Homologue Protein, Myristoylated Alanine-Rich C Kinase Substrate, NF-kappa B p52 Subunit, Nicotinamide-Nucleotide Adenylyltransferase, Nitric Oxide Synthase Type II, Telomerase, Tumor Suppressor Protein p53, Werner Syndrome Helicase, X-ray Repair Cross Complementing Protein 1, Xeroderma Pigmentosum Group A Protein, Proliferating Cell Nuclear Antigen, Promyelocytic Leukemia Protein, Protamines, Clupeine, Salmine, Proto-Oncogene Proteins c-fos, Proto-Oncogene Proteins c-jun, Proto-Oncogene Proteins c-mdm2, Proto-Oncogene Proteins c-myc, Proto-Oncogene Proteins c-rel, ran GTP-Binding Protein, Retinoblastoma Binding Proteins, E2F1 Transcription Factor, Retinoblastoma-Binding Protein 1, Retinoblastoma-Binding Protein 2, Retinoblastoma-Binding Protein 4, Retinoblastoma-Binding Protein 7, Retinoblastoma-Like Protein p107, Retinoblastoma-Like Protein p130, Retinoblastoma Protein, Silent Information Regulator Proteins, Saccharomyces cerevisiae, Thyroid Nuclear Factor 1, and Tumor Protein p73.

In some embodiments, the nuclear-localized reporter gene comprises a gene encoding H2B fused to at least one fluorescent reporter gene. In some embodiments, the nuclear-localized reporter gene comprises a gene encoding an LRG protein, or fragment thereof, fused to a nuclear-localized fluorescent reporter. In some embodiments, the nuclear-localized reporter gene comprises a gene encoding an LGR5-H2B-GFP fusion protein, such that GFP is present and detectable in cells expressing endogenous LGR5. In some embodiments, the nuclear-localized reporter gene comprises a gene encoding an LRG4-H2B-GFP fusion protein, such that GFP is present and detectable in cells expressing endogenous LRG4. In some embodiments, the nuclear-localized reporter gene comprises a gene encoding an LRG6-H2B-GFP fusion protein, such that GFP is present and detectable in cells expressing endogenous LRG6. In some embodiments, an LRG promoter (e.g., LRG4 promoter, LGR5promoter, or LRG6 promoter), or a fragment thereof, is present and drives expression of the transgene, independent of the presence or absence of one or more portions of the LRG gene itself (e.g., introns, exons, or fragments thereof). In other embodiments, the LRG promoter, or a fragment thereof, is present and drives expression of the transgene, and expression of the transgene may involve one or more portions of the LRG gene itself (e.g., introns, exons, or fragments thereof), which may act as enhancer elements (e.g., an LRG gene, or a portion thereof, fused to a nuclear-localized fluorescent reporter gene, or a portion thereof).

In some embodiments, a gene encoding an LGR5-H2B-GFP fusion protein comprises a gene encoding H2B-GFP downstream of the LGR5 ATG start site. In some embodiments, the gene encoding the LGR5-H2B-GFP fusion protein comprises a gene encoding H2B-GFP downstream of the LGR5 ATG start site, and upstream of intron 1 of LGRS (e.g., within exon 1). In some embodiments, the gene encoding the LGR5-H2B-GFP fusion protein does not contain an IRES site. In some embodiments, the gene encoding the LGR5-H2B-GFP fusion protein comprises one or more fragments of SEQ ID NO: 2, such as one or more fragments of the sequence surrounding the genomic insertion site.

In some embodiments, the transgene used to generate the animal models of the present disclosure includes one or more fragments of SEQ ID NO: 2 (e.g., one or more sequences surrounding the genomic insertion site) or sequences with at least 80% homology to these fragments of SEQ ID NO: 2. In some embodiments, the transgene used to generate the animal models of the present disclosure includes one or more fragments of SEQ ID NO: 2 (e.g., one or more sequences surrounding the genomic insertion site) or sequences that are at least 80%, 85%, 90%, 95%, 96%, 97%, 98%, or 99% identical to one or more fragments of SEQ ID NO: 2. In some embodiments, variants of the transgene include one or more portions of an LRG coding region, and/or one or more portions of an LRG non-coding region (e.g., comprising gene expression regulatory elements). In some embodiments, variants of the transgene include neither a portion of an LRG coding region, nor a portion of an LRG non-coding region.

In some embodiments, conservative or non-conservative substitutions are made. For example, it is contemplated that isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, histidine, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic hydroxyl; (4) aromatic (phenylalanine, tyrosine, tryptophan); (5) amide (asparagine, glutamine); and (6) sulfur containing (cysteine and methionine) (e.g., Stryer ed., Biochemistry, pg. 17-21, 2nd ed, WI-I Freeman and Co., 1981). “Non-conservative” changes (e.g., replacement of a glycine with a tryptophan) are also contemplated. Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.).

In some embodiments, at least one cell type or tissue type that expresses the endogenous LGR gene is a stem cell. In some embodiments, the at least one cell type or tissue type that expresses the endogenous LGR gene comprises skin, eye, inner ear, gastrointestinal track, uterus, ovary, prostrate, mammary gland, kidney, liver, pancreas, cervix, and/or placenta cell types or tissue types, In some embodiments, one or more transgene-expressing cells of these tissue types can be isolated, and a cell line can be established. In some embodiments, the cell line is a primary cell line or an immortalized cell line.

Embodiments of the present disclosure also include a method of screening an intervention for a disease or condition. In accordance with these embodiments, the method can include contacting a transgenic animal described herein, or a cell or cell line of derived from a transgenic model descried herein, with a candidate intervention. The method can include determining an effect of the intervention on a disease or condition in the transgenic animal (or cell derived therefrom). In some embodiments, the disease or condition is imparted to the transgenic animal via genetic or chemical means.

In some embodiments, the intervention is selected from the group consisting of a drug, a lifestyle change, an alternative medicine therapy, or a combination thereof. In some embodiments, the disease or condition is cancer. In some embodiments, the disease or condition is associated with stem cell function. In some embodiments, the disease or condition is an intestinal, hepatic, renal, lung, or skin disease or injury.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6,0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “animal” refers to any animal (e.g., a mammal), including, but not limited to, humans, non-human primates, pigs, rodents (e.g., mice, rats, etc.), flies, and the like.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

The term “transgene” as used herein refers to a foreign, heterologous, or autologous gene and/or fragment thereof that is placed into an organism (e.g., by introducing the gene into newly fertilized eggs or early embryos). The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “transgenic animal” refers to any animal containing a transgene.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), hiolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxylmethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-mnethylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylinethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences for the production of a polypeptide, precursor, or RNA (e.g., rRNA, tRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example, a 24-residue oligonucleotide is referred to as a “24-mer.” Oligonucleotides can form secondary and tertiary structures by self-hybridizing or by hybridizing to other polynucleotides. Such structures can include, but are not limited to, duplexes, hairpins, cruciforms, bends, and triplexes.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Transgenic Reporter Systems and Methods of Use

Embodiments of the present disclosure include the use of the transgenic reporter systems provided herein, including the LGR5-H-2B-GFP reporter system. In accordance with these embodiments, the methods include using the transgenic models of the present disclosure in the context of a disease or condition, such as a disease related to stem cell biology and/or cancer. For example, the transgenic models of the present disclosure can be used to investigate skin regeneration and/or wound healing (e.g., as a result of a burn). In some embodiments, the LGR5-H2B-GFP reporter system can be used to track the progress of LGR5-expressing stem cells after the transgenic model (or cells isolated from the transgenic model) is subjected to an intervention involving damage to the skin. Similarly, the transgenic model can be used to investigate the efficacy of various therapies designed to reduce or treat the skin damage. For example, the H2B nuclear localization tag (chromatin-specific) is particularly useful, as it allows for lineage tracing studies and investigations into chromosome dynamics by identifying the timing of when LGR5 gene expression is activated or deactivated.

In some embodiments, the transgenic models of the present disclosure can be used to investigate cancer progression and cancer therapy, such as, but not limited to, ovarian cancer, colorectal cancer, hepatocellular carcinoma, basal cell carcinoma, breast cancer, and esophageal adenocarcinoma, among others. In some embodiments, the LGR5-H2B-GFP reporter system can be used to track the progress of LGR5-expressing stem cells after the transgenic model (or cells isolated from the transgenic model) is subjected to an intervention that causes cancer in the model (e.g., genetic manipulation). Similarly, the transgenic model can be used to investigate the efficacy of various therapies designed to reduce or treat the cancer. As would he recognized by one of ordinary skill in the art based on the present disclosure, other diseases or conditions involving LGRs can be investigated using the transgenic models of the present disclosure.

In some embodiments, adeno-associated viruses designed to mutate the BRCA-1 and/or BRCA-2 gene and/or p53 gene can be injected into the mammary glands of transgenic animals. In addition, chemical induction methods can also be tested. If LGR5 is indeed a cancer stem cell, the tumors would be positive for transgene expression (e.g., eGFP) and may or may not be clonal. If tumors are induced, the animals would he used for studying tumor progression and identifying early stage biomarkers; studying tumor progression, biology of tumor growth, and transcriptomics of tumor to understand molecular mechanisms; and understanding the role of mesenchymal LGR5 cells in tumor progression in a tissue-specific manner.

In some embodiments, one or more transgene positive cells from the transgenic models provided herein can be isolated and cultured, either as primary cells or as immortalized cells. This facilitates in vitro experiments that are more difficult to conduct in vivo, such as experiments to identify modulators of a disease (e.g., proteins, peptides, peptidomimetics, peptoids, small molecules or other drugs). In some embodiments, the transgenic models provided herein can be used to identify and isolate LGR5 stem cells from various tissues, for example, based on GFP expression from an LGR5 transgene (see Example 3). In some embodiments, these isolated stem cells can be cultured in a suitable matrix and under suitable conditions to form organoids, which can provide a 3D model that more accurately mimics tissue architecture than 2D cell culture.

The transgenic models of the present disclosure can he generated via a variety of methods. In some embodiments, CRISPR/Cas 9 systems are used to generate transgenic animals (see e.g., Zhang F, Wen Y, Guo X (2014) Human Molecular Genetics. 23 (R1): R40-6). In some embodiments, embryonal cells at various developmental stages are used to introduce transgenes for the production of transgenic animals. Different methods are used depending on the stage of development of the embryonal cell. The zygote is the best target for micro-injection. The use of zygotes as a target for gene transfer has a major advantage in that in most cases the injected DNA will be incorporated into the host genome before the first cleavage (Brinster et al., Proc. Natl. Acad. Sci. USA 82:4438-4442 (1985)). As a consequence, all cells of the transgenic non-human animal will carry the incorporated transgene. This will in general also be reflected in the efficient transmission of the transgene to offspring of the founder since 50% of the germ cells will harbor the transgene. Additional methods for generating transgenic animal are described, for example, in Palmiter, Ann. Rev. Genet. 20:465-99 (1986); Gordon, Methods in enzymology, vol. 225; and Camper, Biotechniques, Vol 5. No 7. (1987).

In other embodiments, retroviral infection can be used to introduce transgenes into a non-human animal. In some embodiments, the retroviral vector is utilized to transfect oocytes by injecting the retroviral vector into the perivitelline space of the oocyte. In other embodiments, the developing non-human embryo can be cultured in vitro to the blastocyst stage. During this time, the blastomeres can be targets for retroviral infection (Janenich, Proc. Natl. Acad. Sci. USA 73:1260 (1976)). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. (1986)). The viral vector system used to introduce the transgene is typically a replication-defective retrovirus carrying the transgene (Jahner et al., Proc. Natl. Acad Sci. USA 82:6927 (1985)). Transfection is easily and efficiently obtained by culturing the blastomeres on a monolayer of virus-producing cells (Stewart, et al., EMBO J., 6:383 (1987)), Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 (1990), and Haskell and Bowen, Mol. Reprod. Dev., 40:386 (1995)).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 (1981); Bradley et al., Nature 309:255 (1984); Gossler et al., Proc. Acad. Sci. USA 83:9065 (1986); and Robertson et al., Nature 322:445 (1986)). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 (1988)). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in, for example, U.S. Pat. No. 5,614,396.

### 3. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

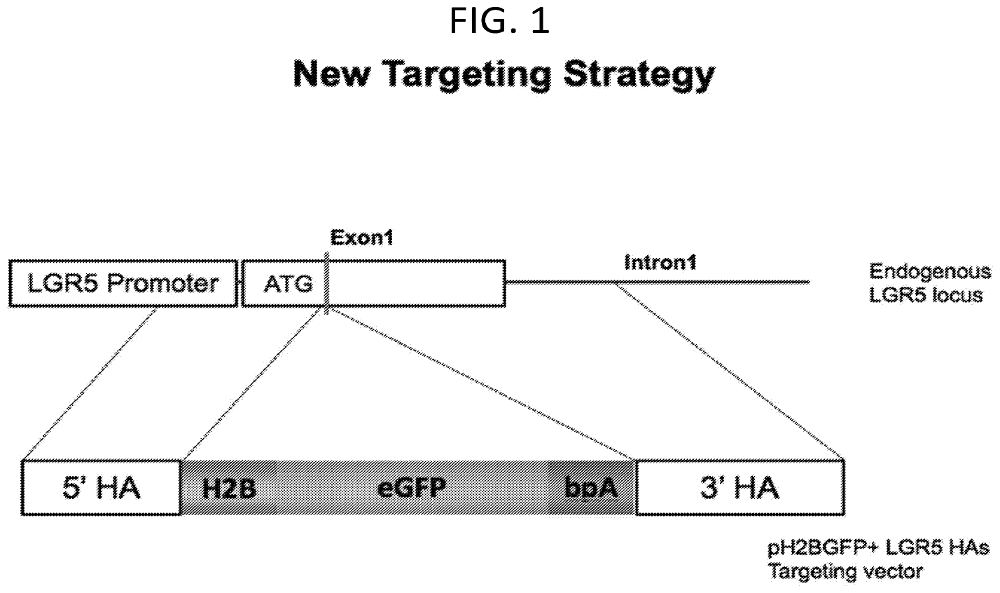

Generation of LGR5-H2B-GFP transgenic model. As shown in FIG. 1, embodiments of the present disclosure include an H2B-GFP transgene inserted into a region of the endogenous LGR5 gene locus. In one embodiment, an H2B-GFP fusion construct was inserted into exon 1 of LGR5, downstream of the ATG start site and upstream of intron 1, and expression of the transgene is driven by the endogenous LGR5 promoter.

Shown below is a representative schematic diagram of a construct used to make the transgenic models, according to one embodiment of the present disclosure, including the corresponding insertion sites of the transgene.

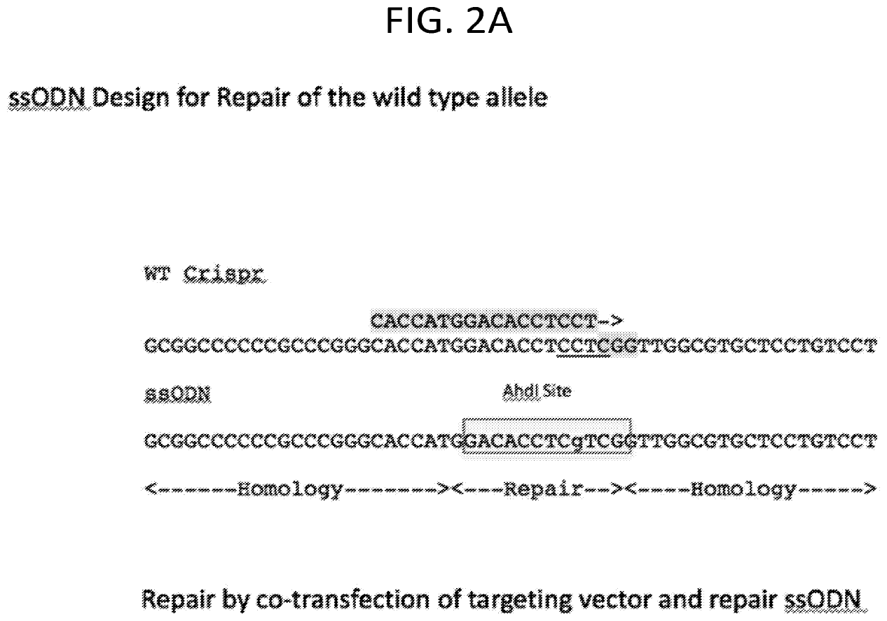

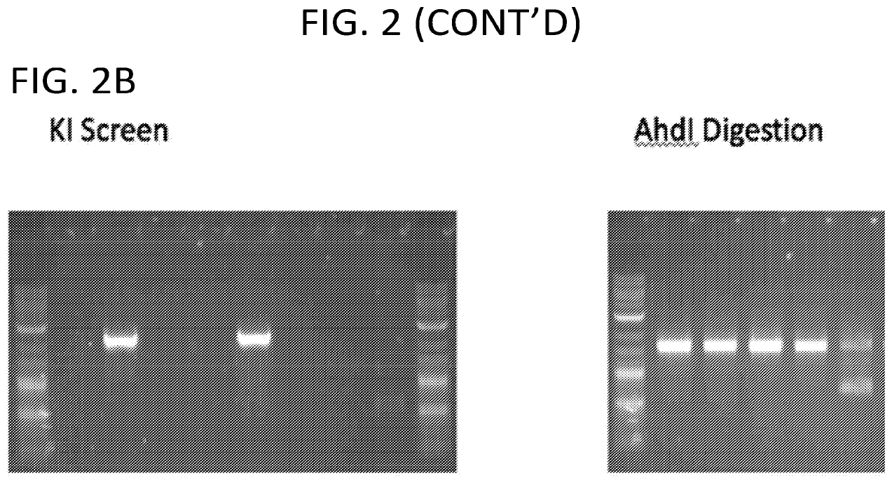

The polynucleotide sequence corresponding to this construct is provided as SEQ ID NO: 1. The polynucleotide sequence of the LGR5-H2B-GFP knock-in, including the genomic insertion sites, is represented by SEQ ID NO: 2. A transgenic reporter was generated by linking the porcine H2B and UP coding sequences. Additionally, 5′ and 3′ homology arms were added so as to target the transgene to the 5′ end of the pig endogenous LGR5 gene. The design placed the incoming reporter just downstream of the LGR5 ATG sequence. Using nucleofection, the targeting plasmid and a CRISPR-Cas9 with guide RNA were introduced into the pig LGR5 just downstream of the ATG signal. The cells used were porcine fetal fibroblasts. Fibroblasts were seeded as single cell clones and colonies were identified that had the correct insertion (FIG. 2B). Those cell lines were used for somatic cell nuclear transfer to produce the transgenic pigs.

Previous attempts to generate an LGR transgenic reporter model used a Bacterial Artificial Chromosome containing all the LGR5 regulatory regions and a fluorescent tag inserted into a region of the gene. The BAC was introduced into cell by nucleofection and the resulting cells used for SCNT. Transgenic pigs containing the BAC were analyzed and none expressed the reporter as expected. Further analysis indicated that the BAC had rearranged making the reporter inactive. Additionally, an approach was used that was previously used in mice (see, e.g., Morita et al., Mol Cell Bio; 24:9736-9743 (2004)), which introduced an internal ribosomal entry site (IRES) linked to a cytoplasmic GFP and inserted this reporter into the 3′ UTR after Exon 18. This reporter was introduced into the pig LGR5 locus using TALEN's induced homology directed repair and generated multiple offsprina. Progeny was extensively analyzed, and it was determined that the reporter was active but was of little utility as it only mirrored the endogenous LGR5 pattern in some tissues.

Consequently, the H2B-GFP was knocked-in and simultaneously the opposite allele was corrected/protected using CRISPR-CAS HDR and a single strand DNA carrying the wild type LGR5 sequence. Essentially, this system was designed to turn the INDEL in the opposite allele into a wild type allele (FIG. 2A). Additionally, a screen identified two colonies containing the knock in (left panel) and one of them also carrying the corrected allele (lane 6), demonstrating the efficacy of this approach (FIG. 2B). This has been confirmed using sequencing and by generating additional colonies.

Therefore, as provided herein, the transgenic models of the present disclosure include several alterations from past models and from currently existing models, including, but not limited to: removing the IRES that contributed to gene silencing in some tissues; moving the reporter to the 5′ end to create a LGR5-H2B-GFP fusion protein; and moving from the cytoplasmic to the porcine H2B-GFP chromatin/nuclear label.

### Example 2

Gene expression patterns in LGR5-H2B-GFP transgenic model. As shown in FIGS. 3 and 4, transgene expression in these lines consistent with endogenous LGR5 expression, contained bright and easily scoreable nuclear-localized GFP expression in LGR5-expressing cells, including, for example, the duodenum, the jejunum, the skin and biliary tree. Expression in other cells and tissue types Ivere also observed (data not shown).

LGR5-expressing stem cells play key roles in many organs; thus, each organ can be a potential clinical target. LGR5 expression has been reported in skin, eye, inner ear, gastrointestinal track, uterus, ovary, prostrate, mammary gland, kidney, liver, pancreas, cervix, and/or placenta cell types or tissue types. And in each of these tissues there are associated disorders, some of which may be dependent on LGR5 cells (e.g., cancer). In addition, LGR5 cells are activated upon injury so the transgenic models provided herein can be used to elucidate how each tissue deals with injury, which will help determine the role of LGR5 cells and to find new targets of therapeutic development.

Finally, transgenic pig models have unique characteristics that make them more amenable to developing therapeutics that can be successful in a human population. From a regulatory FDA perspective, it may be possible to do both safety and efficacy testing in these animals and reduce the burden needed to initiate human clinical trials.

### Example 3

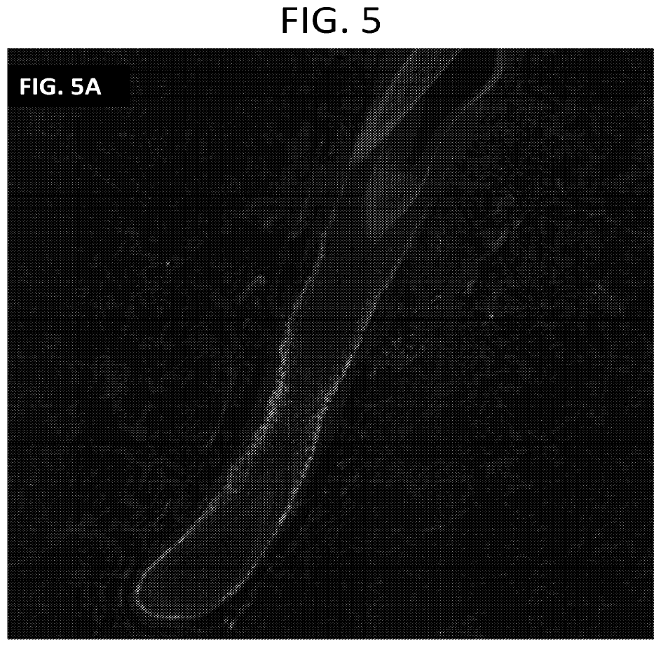

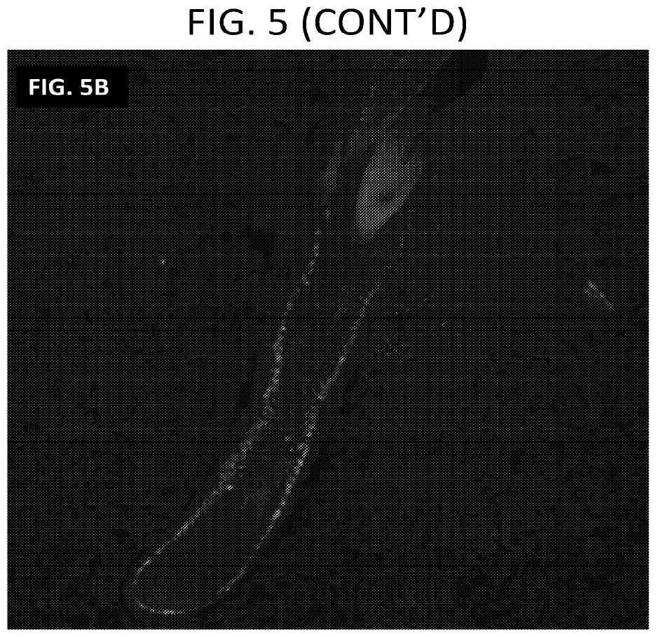

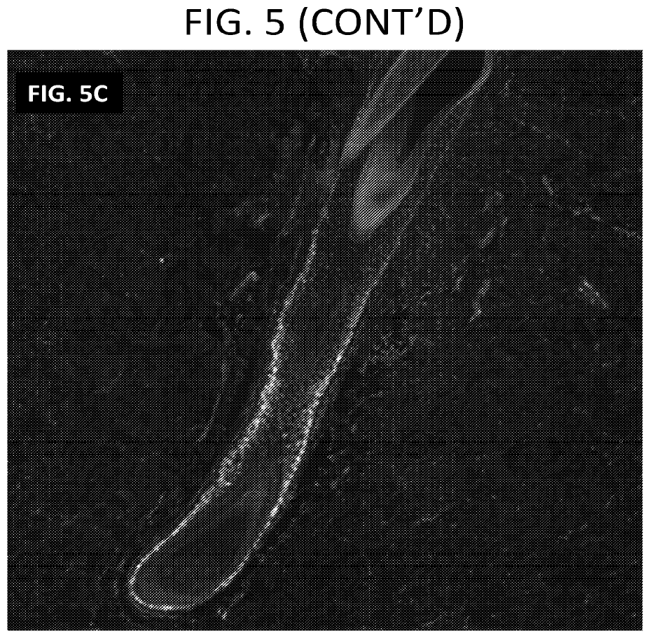

As shown in FIGS. 5A-5C, experiments were conducted to investigate whether expression of endogenous LGR5 mRNA co-localizes with LGR5-H2B-GFP transgene protein expression. Results clearly demonstrate that endogenous LGR5 RNA transcripts (FIG. 5B) were co-expressed in the same location as H2B-GFP (FIG. 5A). RNA Scope for a pig-specific LGR5 probe shows that LGR5 RNA transcript can be detected in the same location that LGRS driven H2B-GFP is expressed in the outer-root sheath of the hair follicle in the skin. (FIG. 5A, anti-GFP antibody; FIG. 5B, RNA-Scope (in situ hybridization) probe specific to porcine LGR5; FIG. 5C, merged images)

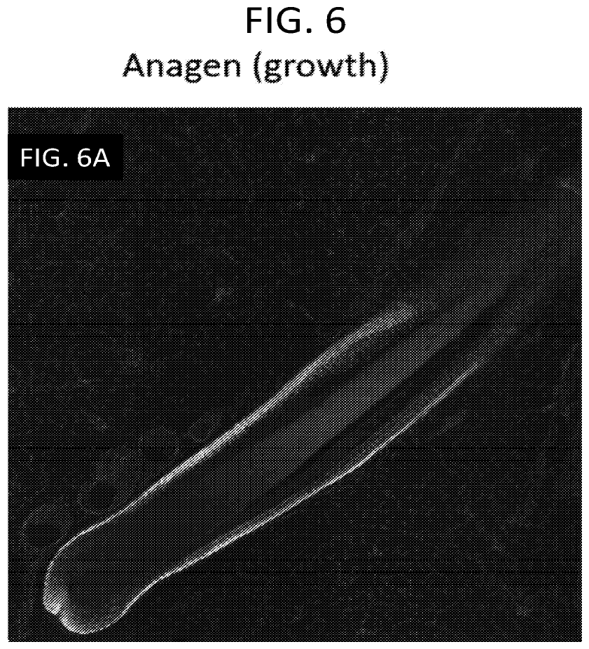

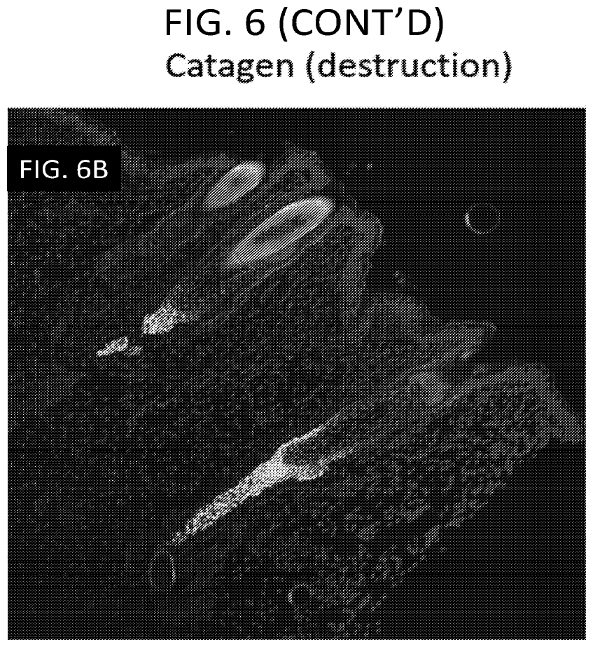

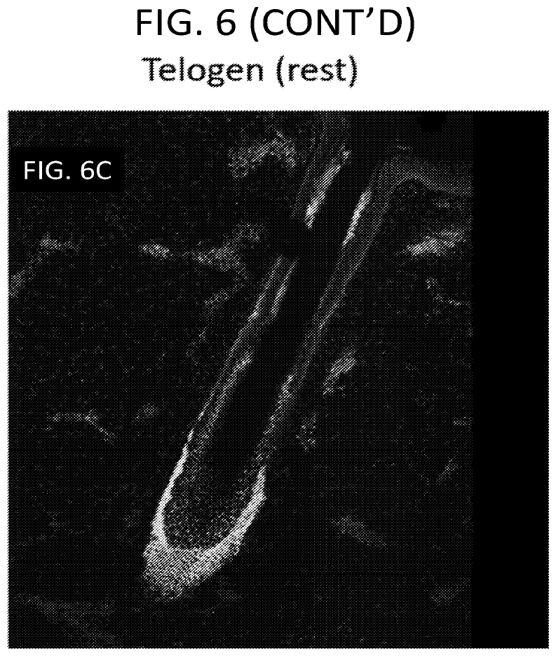

Experiments were also conducted to investigate whether endogenous LGR5-H2B-GFP transgene protein expression is consistent with endogenous LGR5 protein expression. As shown in FIGS. 6A-6C, results clearly demonstrate that LGR5-H2B-GFP transgene is expressed in locations consistent with endogenous LGR5 expression in various stages of the hair growth cycle, including during anagen (FIG. 6A), catagen (FIG. 6B), and telogen (FIG. 6C) stages. FIGS. 6A-6C include representative 20× confocal microscopy images of dorsal skin from a one-week old transgenic piglet. Green: nuclear H2B-GFP, blue: DAPI. LGR5 expressing cells (nuclear green) are found within the hair follicle, but not in any other location throughout the epidermis. This model faithfully recapitulates the expected position of LGR5 stem cells within the stages of the hair cycle.

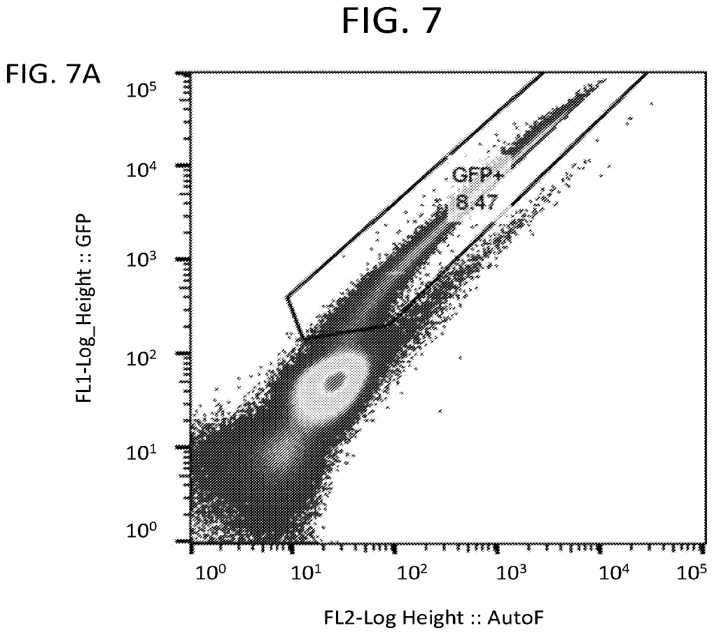

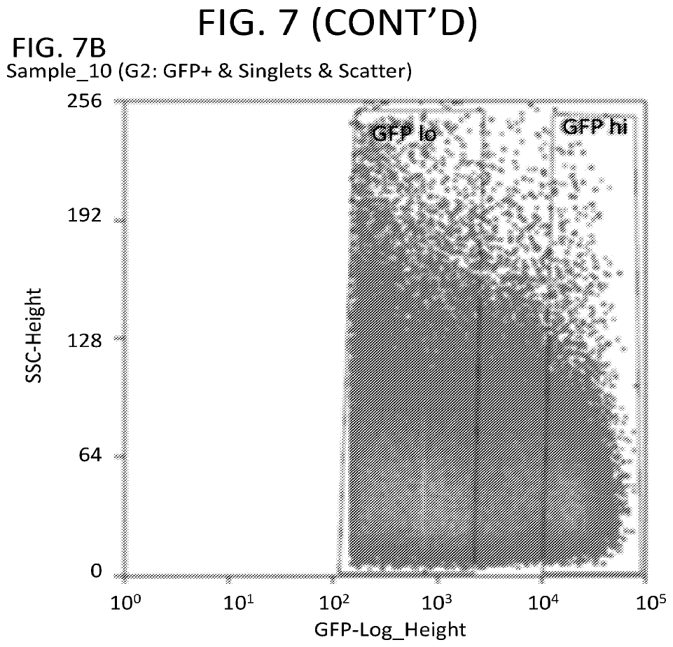

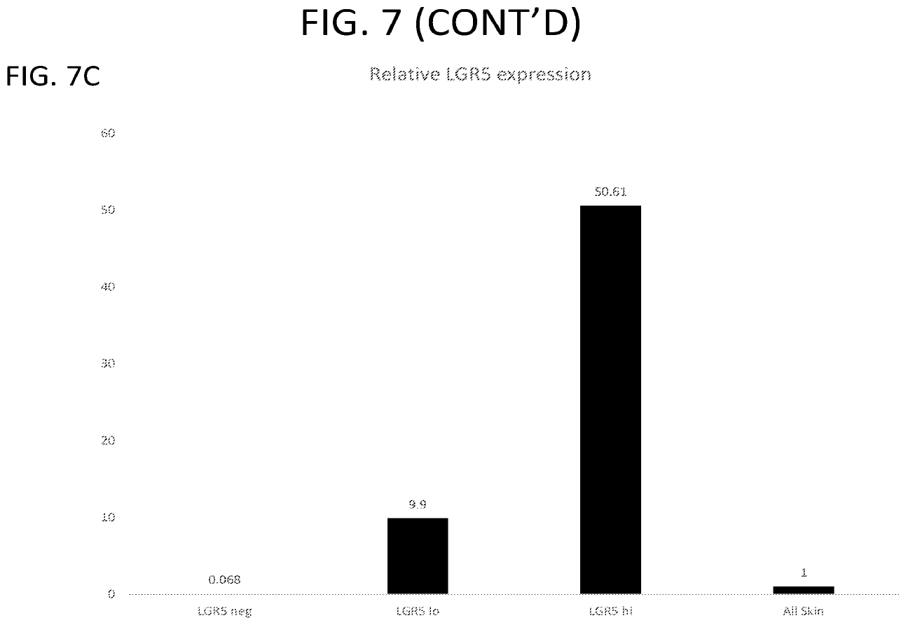

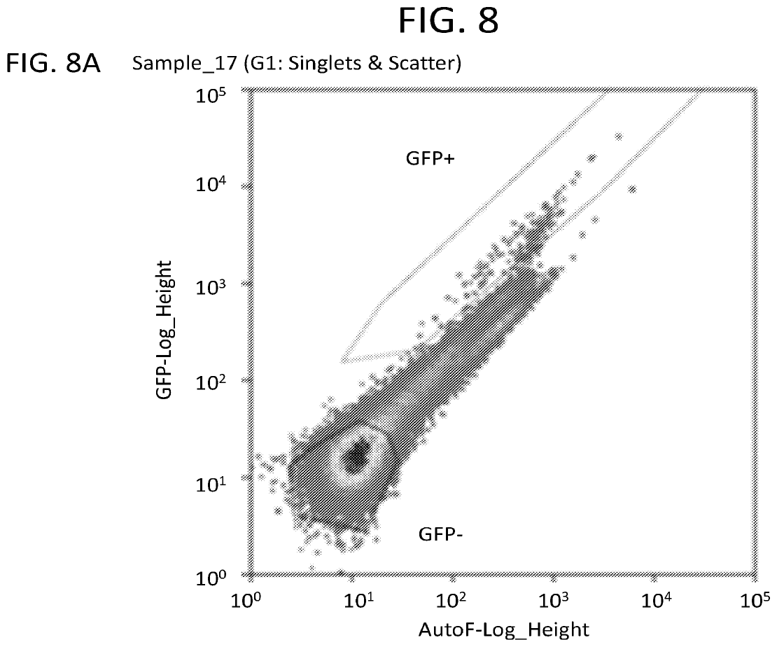

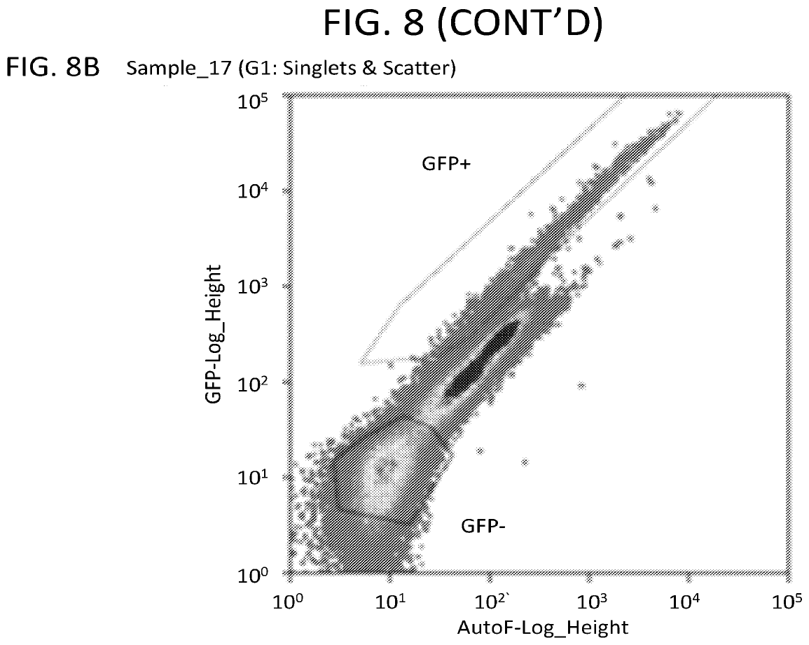

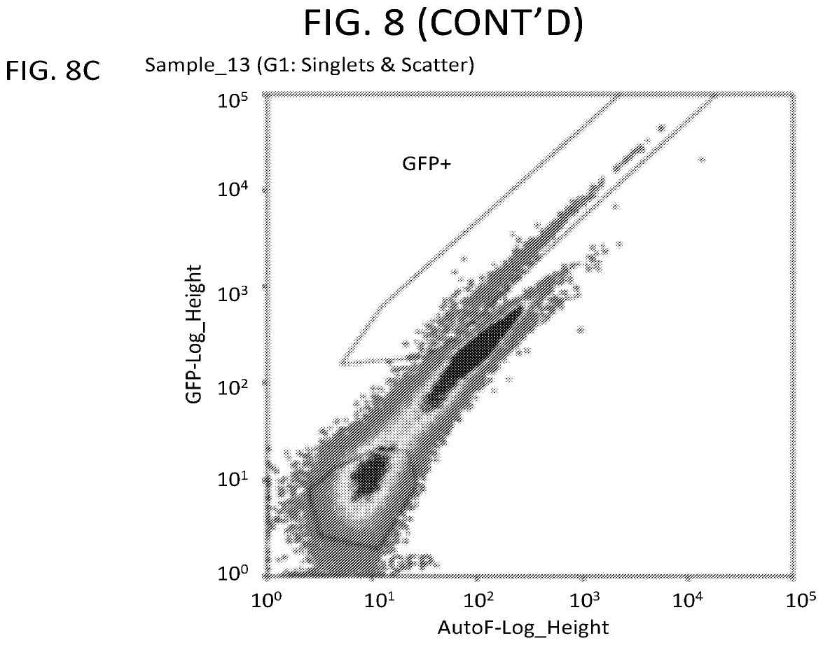

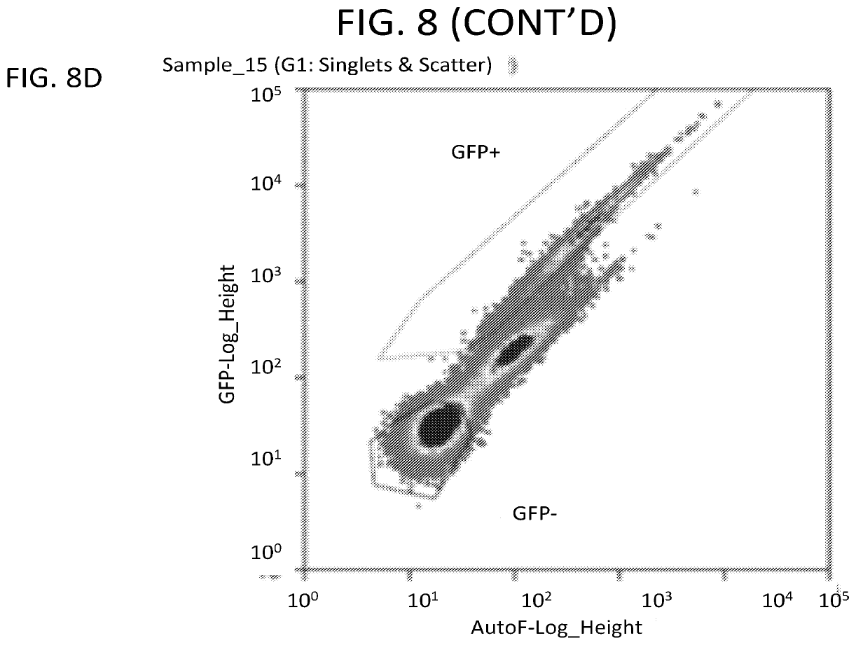

As shown in FIGS. 7A-7C, GR5-H2B-GFP transgene expression in individual epidermal cells also correlates with endogenous LGR5 mRNA expression. Single cell epidermis was isolated from LGR5-H2B-GFP samples and sorted based on GFP expression. Each sample was analyzed for levels of endogenous LGR5 mRNA expression by RT-qPCR. Values are normalized to GAPDH and RNA extracted from all components of skin in a delta-delta CT analysis. Additionally, FIGS. 8A-8D includes representative scatter plots from FACS sorting of GFP-positive stem cells expressing the LGR5-H2B-GFP transgene in liver (FIG. 8A), biliary tree (FIG. 8B), pancreas (FIG. 8C), and small intestine (FIG. 8D), which demonstrates that LGR5-positive stem cells can be obtained from various tissues of the transgenic animals described further herein.

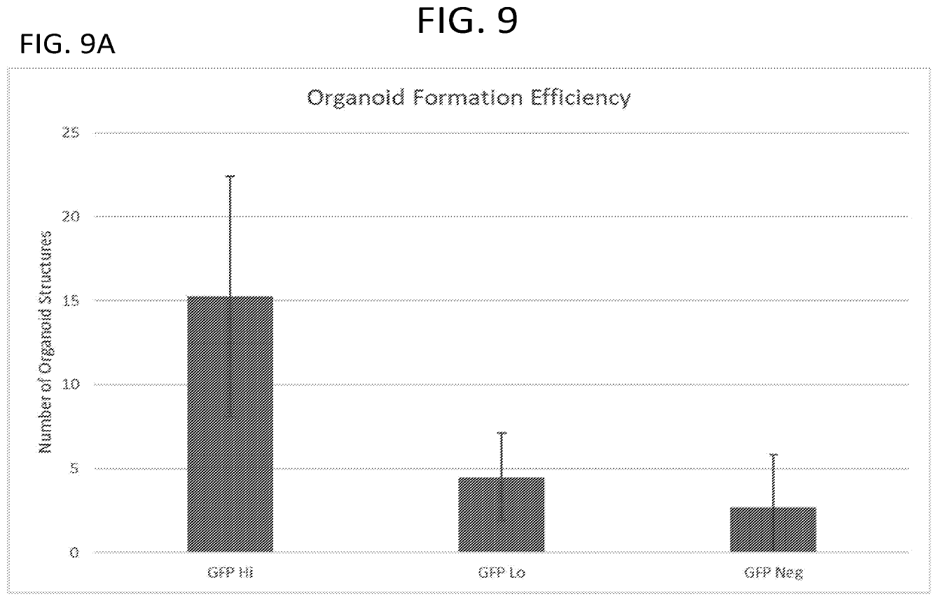

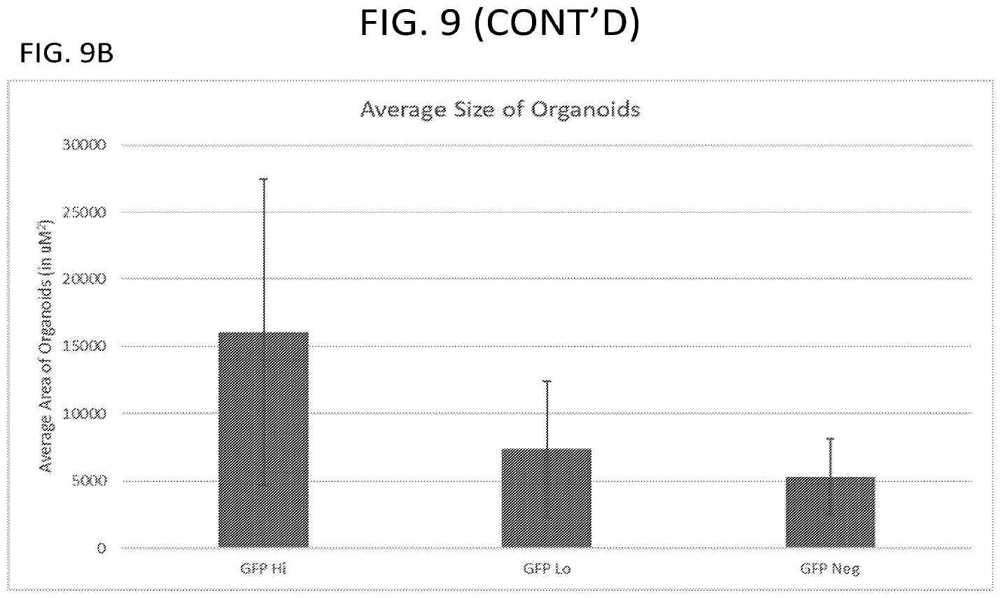

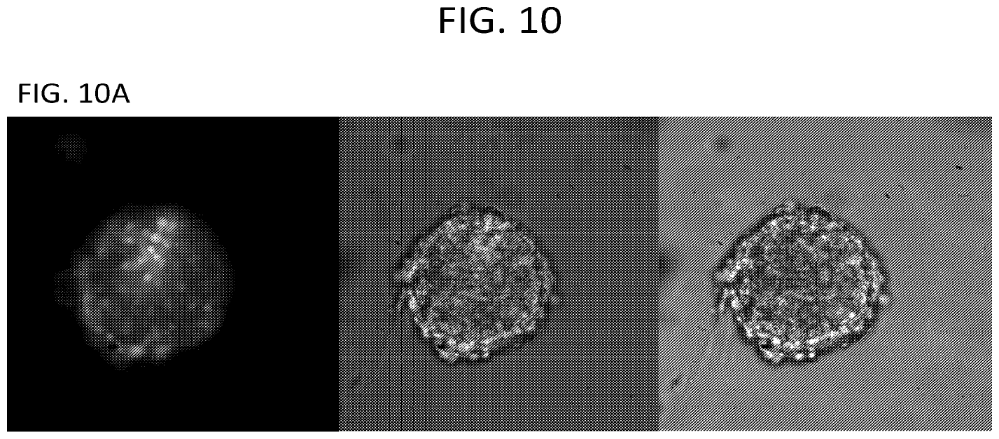

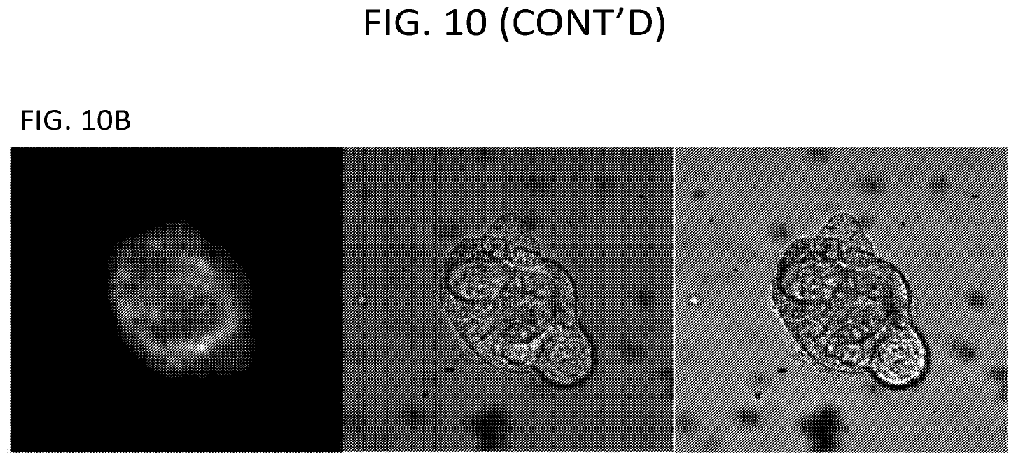

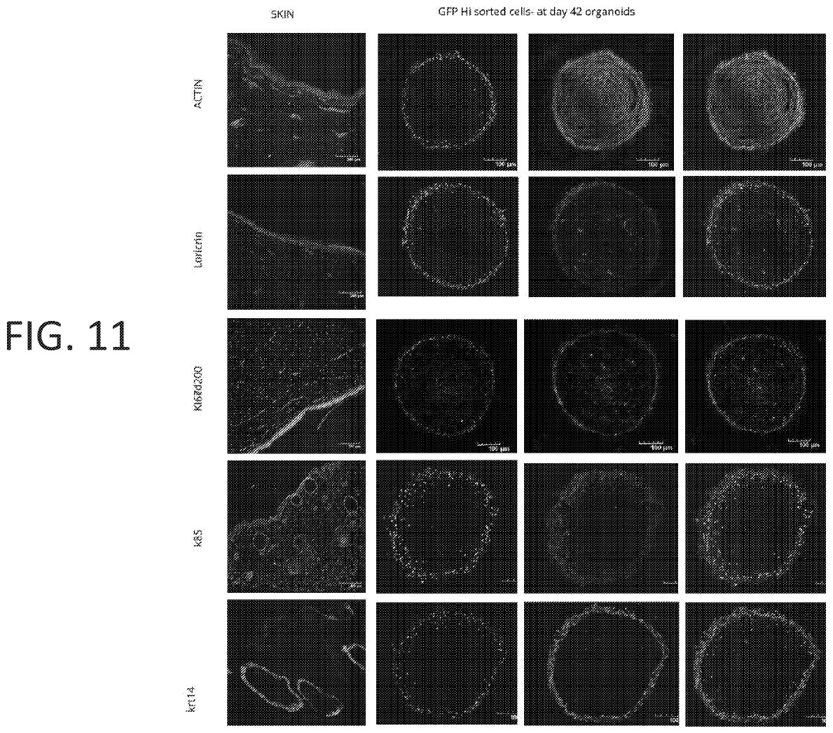

Because the LGR5 transgenic animals of the present disclosure facilitate the isolation of LGR5 stem cells, experiments were conducted to test the ability of these stem cells to thnn organoids. As shown in FIGS. 9A-9B, representative bar graph results demonstrate the ability to from organoids with efficiency (FIG. 9A) and proper size (FIG. 9B) from LGR5-UP-positive hair follicle stem cells. Cells were isolated from LGR5-H2B-GFP transgene containing porcine epidermis and single cell sorted based on fluorescence. Cells were plated in organoid conditions in Matrigel containing growth factors including Wnt and R-spondin. After passaging, organoids derived from LGR5-H2BGFP-expressing epidermal cells were able to form organoids. Additionally, as shown in FIGS. 10A-10B, LGR5-GFP-positive cells plated in Matrigel containing growth factors formed skin organoids (FIG. 10A) and biliary tree organoids (FIG. 10B). Natural nuclear GFP expression in a fluorescent microscope is shown in the left image, brightfield images are in the center, and merged images are on the right. Images were obtained after 12 days in culture and display markers of proliferation, epidermis, and stem cells (data not shown but can be available upon request). Additionally, as shown in FIG. 11, day 42 organoids were characterized using various epidermal markers. Each panel shows a cryosection of an organoid stained with DAPI, the indicated marker (ACTIN, Loricrin, KI67cd200 k85, and krt14), or merged. These results demonstrate that organoids form cellular layers with positive marker expression in the epidermis toward the exterior of the organoid, whereas intracellular portions of the organoid contained keratinized cells (no nuclei and ECM).

### Example 4

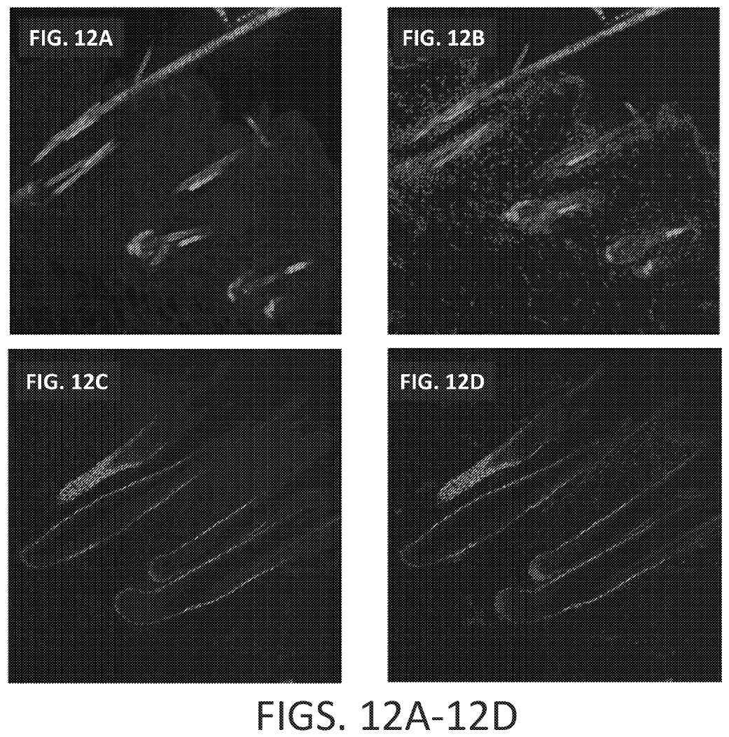

Experiments were also conducted to compare LGR5-GFP expression in an existing mouse model and a porcine model. FIGS. 12A-12D include representative images of LGR5-GFP expression in a mouse model (FIGS. 12A-12B) as compared to a porcine model (FIGS. 12C-12D). GFP expression is overlaid with DAPI staining in FIGS. 12B and 12D. Confocal microscopy of cryosections from telogen stage mouse skin (naturally synchronized) from LGR5-IRES-GFP model skin. GFP fluorescence is detected in a few cells at the base of the hair follicle (hair is auto-fluorescent). Cross section of hairs from LGR5-H2B-GFP porcine skin demonstrating several stages of the hair cycle (naturally asynchronous). Nuclear GFP allows for tracking of cells at single cell resolution.

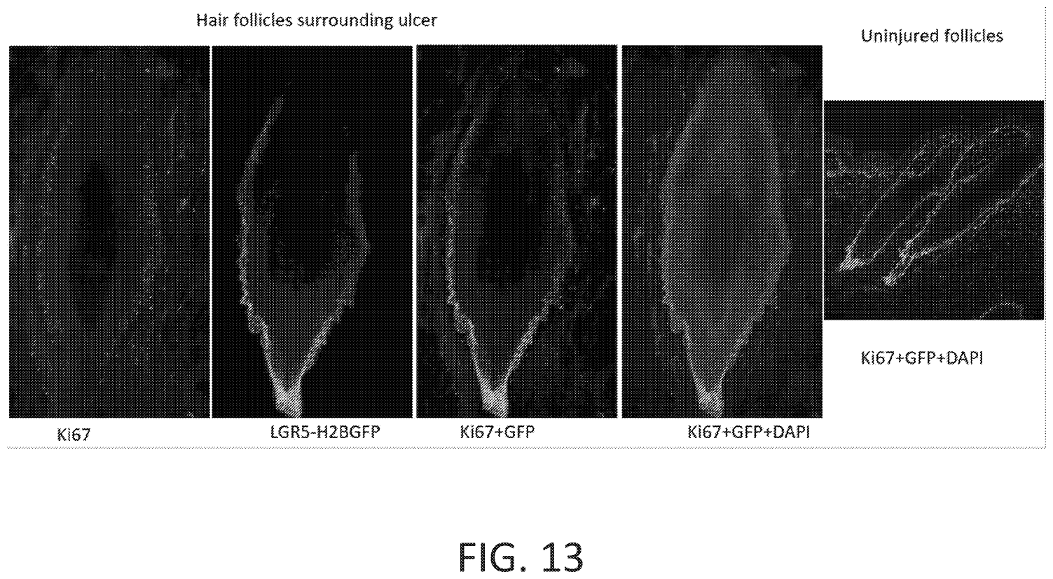

In addition to successful LGR5-GFP expression in a porcine model, experiments were conducted to assess expression in the context of cell proliferation. As shown in FIG. 13, hair follicles surrounding a wound displayed increased proliferation. LGR5-GFP+ hair follicle cells from skin within 1 cm of a chronic ulcer wound show increased staining with Ki-67, a nuclear marker for proliferation. These results demonstrate contribution of the LGR5-GFP stem cells toward re-epithelialization of wounds and the hyper-proliferative response of the LGR5-stem cell populations. Uninjured follicles show minimal co-localization with Ki-67 in the LGR5-GFP stem cells (shown is catagen stage uninjured hair follicle to match catagen injured follicle).

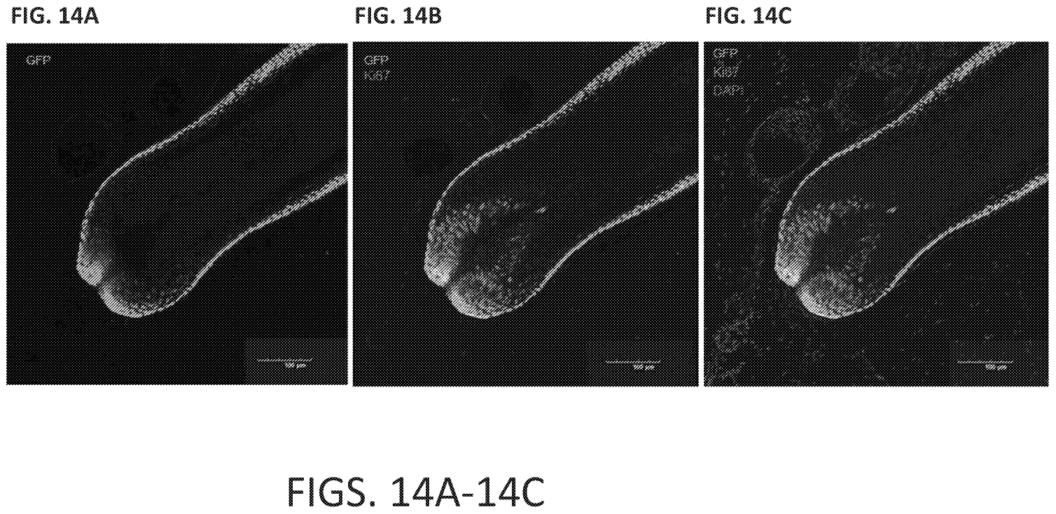

As shown in FIGS. 14A-14C, LGR5-GFP expression was also found in hair germ cells, the primary proliferating hair germ cells in anagen (G-FP expression is overlaid with Ki67 staining FIG. 14B, and DAPI staining in FIG. 14C). Representative images of the base of an anagen stage hair follicle shows stem cell proliferation and H2B-GFP dilution. 20× confocal microscopy shows the hair follicle expressing natural GFP (FIG. 14A), co-stained with the nuclear marker for proliferation Ki-67 (FIG. 14B), and DAPI (FIG. 14C). Scale bar indicates 100 μM. Nuclear GFP allows for single cell resolution and tracking of cell behavior.

What is claimed is:

1. A non-human transgenic animal comprising a genotne that expresses a nuclear-localized reporter gene in at least one cell type or tissue type that also expresses an endogenous leucine-rich repeat-containing G-protein coupled receptor (LGR) gene.

2. The transgenic animal of claim 1, wherein the endogenous LRG gene is leucine-rich repeat-containing G-protein coupled receptor 4 (LGR4), LRG gene is leucine-rich repeat-containing G-protein coupled receptor 5 (LGR5), or LRG gene is leucine-rich repeat-containing G-protein coupled receptor 6 (LGR6).

3. The transgenic animal of claim 1, wherein the nuclear-localized reporter tzene is a fluorescent reporter.

4. The transgenic animal of claim 3, wherein the fluorescent reporter comprises at least one of GFP, eGFP, mCherry, CFP, BFP, YFP, aFP, photoactivatable GFP, dsRed (Discosoma species fluorescent protein), mFruits (mutants of dsRed), TagRFPs (Evrogen), eqFP611 (isolated from sea anemone Entacinctea quadricolor), Dronpa (photoswitchable fluorescent protein), and EosFP (photoconvertable fluorescent protein).

5. The transgenic animal of claim 1, wherein the nuclear-localized reporter gene comprises a gene encoding H2B.

6. The transgenic animal of claim 5, wherein the nuclear-localized reporter gene comprises a gene encoding an H2B-GFP fusion protein.

7. The transgenic animal of claim 1, wherein the nuclear-localized reporter gene comprises a gene encoding an LRG protein, or fragment thereof, fused to a nuclear-localized fluorescent reporter.

8. The transgenic animal of claim 7, wherein the nuclear-localized reporter gene comprises a gene encoding an LGR5-H2B-GFP fusion protein.

9. The transgenic animal of claim 8, wherein the gene encoding the LGR5-H2B-GFP fusion protein comprises a gene encoding H2B-GFP downstream of the LGR5 ATG start site.

10. The transgenic animal of claim 8, wherein the gene encoding the LGR5-H2B-GFP fusion protein comprises a gene encoding H2B-GFP downstream of the LGR5 ATG start site, and upstream of intron 1 of LGR5.

11. The transgenic animal of claim 8, wherein the gene encoding the LGR5-H2B-GFP fusion protein does not contain an IRES site.

12. The transgenic animal of claim 8, wherein the gene encoding the LGR5-H2B-GFP fusion protein comprises one or more fragments of SEQ ID NO: 2.

13. The transgenic animal of claim 1, wherein the at least one cell type or tissue type that expresses the endogenous LGR gene is a stem cell.

14. The transgenic animal of claim 1, wherein the at least one cell type or tissue type that expresses the endogenous LGR gene comprises skin, eye, inner ear, gastrointestinal track, uterus, ovary, prostrate, mammary gland, kidney, liver, pancreas, cervix, and/or placenta cell types or tissue types.

15. A cell or cell line derived from the transgenic animal of claim 1.

16. The cell or cell line of claim 15, wherein the cell line is a primary cell line or an immortalized cell line.

17. The cell or cell ine of claim 15, wherein the cell is a stem cell.

18. An organoid derived from the stem of claim 17.

19. A method of generating an organoid comprising culturing the stem cell of claim 17 in a matrix under conditions sufficient to promote growth and/or proliferation of the stem cell.

20. A method of screening an intervention for a disease or condition, the method comprising:
a) contacting the transgenic animal of claim 1 or the cell or cell line of claim 15 with a candidate intervention; and
b) determining the effect of said intervention on a disease or condition in the transgenic

21. The method of claim 20, wherein the intervention is selected from the group consisting of a drug, a lifestyle change, an alternative medicine therapy, or a combination thereof.

22. The method of claim 20, wherein the disease or condition is cancer.

23. The method of claim 20, wherein the disease or condition is associated with stem cell function.

24. The method of claim 20, wherein the disease or condition is an intestinal, hepatic, renal, lung, or skin disease or injury.
